(19) United States
(12) Patent Application Publication
(10) Pub. No.: US 2024/0335471 A1
(43) Pub. Date: Oct. 10, 2024

(54) VECTOR SYSTEMS FOR DELIVERY OF TWO POLYNUCLEOTIDES AND METHODS OF MAKING AND USING SAME

(71) Applicant: A2 Biotherapeutics, Inc., Agoura Hills, CA (US)

(72) Inventors: Agnes Hamburger, Newbury Park, CA (US); Jee Young Mock, North Hollywood, CA (US); Mark Daris, Newbury Park, CA (US)

(21) Appl. No.: 18/294,039

(22) PCT Filed: Aug. 1, 2022
(86) PCT No.: PCT/US22/39009

§ 371 Date: Jan. 31, 2024

Related U.S. Application Data
(60) Provisional application No. 63/229,008, filed on Aug. 3, 2021.

(51) Int. Cl. A61K 35/17; A61K 39/00; C12N 5/0781; C12N 5/0783; C12N 5/0786; C12N 15/86

(52) U.S. Cl. CPC A61K 35/17; A61K 39/4631; A61K 39/4632; A61K 39/464411; C12N 5/0635; C12N 5/0636; C12N 5/0645; C12N 5/0646; C12N 15/86; A61K 2239/13; A61K 2239/17; A61K 2239/21; A61K 2239/22; C12N 2740/15043; C12N 2750/14143

(57) ABSTRACT

The disclosure relates to a dual vector system with a first vector comprising a sequence encoding an inhibitory receptor and a sequence encoding a first part of an activator receptor; and a second vector, comprising a sequence encoding a second part of the activator receptor.

### BACKGROUND

A variety of genetic vectors are used in the life sciences to delivery polynucleotides to cells, for genetic engineering or other purposes. Many vectors can only deliver relatively small polynucleotide sequences. One strategy to delivery large polynucleotide sequences is to divide the polynucleotide sequence into two polynucleotides, each delivered individually. In such a divided vector system, without a control mechanism, some cells in a cell population may stochastically receive only one of the two polynucleotides. There is an unmet need for vector systems for delivery of two polynucleotides to the same cell that ensure both polynucleotides are delivered to each target cell.

### SUMMARY

The disclosure provides vector systems for delivery of two polynucleotides to the same cell, comprising: (a) a first vector, wherein the first vector comprises a first polynucleotide, the first polynucleotide comprising a sequence encoding an inhibitory receptor and a sequence encoding a first part of an activator receptor, wherein the activator receptor comprises an antigen binding domain, at least a first hinge domain, at least a first transmembrane domain and intracellular domain; and (b) a second vector, wherein the second vector comprises a second polynucleotide, the second polynucleotide comprising a sequence encoding a second part of the activator receptor, whereby the first and second parts of the activator receptor together form a functional activator receptor when co-expressed by a cell.

In some embodiments of the vector system of the disclosure, the first polynucleotide comprises a sequence encoding a self-cleaving polypeptide or an internal ribosome entry site (IRES) between the sequence encoding the inhibitory receptor and the sequence encoding the first part of the activator. In some embodiments, wherein the first polynucleotide comprises a sequence encoding a promoter between the sequence encoding the inhibitory receptor and the sequence encoding the first part of the activator.

In some embodiments of the vector system of the disclosure, the activator receptor is a chimeric antigen receptor (CAR). In some embodiments, the antigen binding domain of the activator receptor comprises a Fab. In some embodiments, the first part of the activator receptor comprises a light chain of the Fab, and the second part of the activator receptor comprises a heavy chain of the Fab, the transmembrane domain and the intracellular domain. In some embodiments, the first part of the activator receptor comprises a heavy chain of the Fab, and the second part of the activator receptor comprises a light chain of the Fab, the transmembrane domain and the intracellular domain.

In some embodiments of the vector system of the disclosure, the first part of the activator receptor comprises a first hinge domain, a first transmembrane domain and the intracellular domain, and the second part of the activator comprises the antigen binding domain, a second hinge domain and a second transmembrane domain. In some embodiments, the first part of the activator comprises the antigen binding domain, a first hinge domain and a first transmembrane domain, and the second part of the activator comprises a second hinge domain, a second transmembrane domain and the intracellular domain. In some embodiments, the first and second hinge domains comprise CD8α hinge domains. In some embodiments, association of the CD8α hinges of the first and second parts of the activator receptor in the cell produces a functional activator receptor.

In some embodiments of the vector system of the disclosure, the first part of the activator receptor comprises a first hinge domain, a first transmembrane domain and the intracellular domain, and the second part of the activator comprises the antigen binding domain, a second hinge domain and a second transmembrane domain. In some embodiments, the first hinge domain comprises a Fos hinge domain, and the second hinge domain comprises a Jun hinge domain. In some embodiments, the first hinge domain comprises a Jun hinge domain, and the second hinge domain comprises a Fos hinge domain. In some embodiments, the Fos hinge further comprises a CD8α hinge sequence comprising one or more substitutions of serines for cysteines. In some embodiments, the Jun hinge further comprises a CD8α hinge sequence comprising one or more substitutions of serines for cysteines. In some embodiments, association of the Fos and Jun hinges in the cell produces a functional activator receptor. In some embodiments, the activator receptor comprises an HLA-A transmembrane domain.

In some embodiments of the vector system of the disclosure, the activator receptor comprises a CD3ζ intracellular domain. In some embodiments, the intracellular domain of the activator receptor comprises one or more co-stimulatory domains. In some embodiments, the one or more co-stimulatory domains comprises an isolated or derived from CD27 molecule (CD27), CD28, CD137, TNF receptor superfamily member 4 (OX40), TNF receptor superfamily member 8 (CD30), CD40 molecule (CD40), CD40 ligand (CD40L), CD3ζ, integrin subunit beta 2 (LFA-1), inducible T cell costimulator (ICOS), CD2 molecule (CD2), CD7 molecule (CD7), TNF superfamily member 14 (LIGHT), killer cell lectin like receptor C2 (NKG2C), CD276 molecule (B7-H3), or hematopoietic cell signal transducer (DAP10).

In some embodiments of the vector system of the disclosure, the activator receptor is a T cell receptor. In some embodiments, the first part of the activator receptor comprises a TCRα chain, and the second part of the activator receptor comprises a TCRβ chain. In some embodiments, the first part of the activator receptor comprises a TCRβ chain, and the second part of the activator receptor comprises a TCRα chain.

In some embodiments of the vector system of the disclosure, the inhibitory receptor comprises an intracellular domain isolated or derived from LILRB1. In some embodiments, the inhibitory receptor comprises a transmembrane domain. In some embodiments, the transmembrane domain is isolated or derived from TCRα, TCRβ, CD8α, CD28 or LILRB1. In some embodiments, the inhibitory receptor further comprises an extracellular hinge domain. In some embodiments, the extracellular hinge domain comprises a hinge domain isolated or derived from CD8α, CD28 or LILRB1. In some embodiments, the inhibitory receptor comprises an antigen binding domain specific to an antigen whose expression is lost in a cancer cell through loss of heterozygosity.

In some embodiments of the vector system of the disclosure, the activator receptor comprises an antigen binding domain specific to a cancer antigen.

In some embodiments of the vector system of the disclosure, the first and/or second polynucleotide comprises a polynucleotide sequence encoding an additional gene. In some embodiments, the additional gene comprises a marker, a reporter, a short interfering RNA, or an inducible kill switch.

In some embodiments of the vector system of the disclosure, the first and second vectors are plasmids. In some embodiments, the first and second vectors are viral vectors. In some embodiments, the viral vectors are adeno-associated viral (AAV) viral vectors or retroviral vectors. In some embodiments, the retroviral vectors are lentiviral vectors.

The disclosure provides a cell, comprising the vector system described herein. In some embodiments, the cell expresses a functional activator receptor and a functional inhibitory receptor. In some embodiments, the cell is an immune cell.

The disclosure provides pharmaceutical compositions comprising the cells of the disclosure, and a pharmaceutically acceptable carrier, diluent, or excipient.

The disclosure provides methods of treating cancer in a subject, comprising administering a therapeutically effective amount of the cells or pharmaceutical composition of the disclosure.

The disclosure provides methods of making a recombinant immune cell, comprising: (a) providing a plurality of immune cells; and (b) transforming the plurality of immune cells with the vector system of the disclosure.

### BRIEF DESCRIPTION OF THE DRAWINGS

FIG. 1 is a diagram illustrating conventional methods to introduce two genes into a cell for co-expression.

FIG. 2 is a diagram illustrating an embodiment of a vector system according to the present disclosure.

FIG. 3 is a diagram illustrating an embodiment of a vector system according to the present disclosure, which is a dual vector system encoding a split activator chimeric antigen receptor (CAR) with a Fab antigen binding domain and an inhibitory (blocker) scFv CAR receptor. The activator CAR is split between the two vectors via the ligand binding domain (LBD) of a Fab. One vector contains (construct 1, top) encodes the scFv CAR blocker and the light chain (LC) of an activator CAR. The second vector (construct 2, bottom) contains the VH-CH1, hinge, transmembrane and intracellular domains of the activator CAR. The second vector has additional space that can be used to encode additional genes in its payload.

FIG. 4A is a plot (left) and table (right) showing that a split CD19 CAR retains activator function, and is effectively blocked when co-transfected with an HLA-A*02 blocker (A2) using the dual vector system of the disclosure.

FIG. 4B are a series of fluorescence activated cell sorting (FACS) plots showing expression of CD19 activator and HLA-A*02 blocker receptor target antigens on target Jurkat cells.

FIG. 4C are a series of fluorescence activated cell sorting (FACS) plots showing expression of CD19 activator and HLA-A*02 blocker receptor target antigens on effector Jurkat cells.

FIG. 5A is plot (left) and a table (right) showing that a split EGFR CAR retains function, and is effectively blocked when co-transfected with an HLA-A*02 (A2) blocker using the dual vector system of the disclosure.

FIG. 5B is a series of FACS plots showing expression of EGFR activator and HLA-A*02 blocker receptor target antigens on target Jurkat cells, and the expression of the activator and blocker receptors on effector Jurkat cells.

FIG. 6 is a pair of plots showing that a split MSLN CAR is reconstituted and expressed in cells transfected with the MSLN CAR split between two vectors (left). Expression is compared to that of cells co-transfected with the split MSLN CAR/blocker on two vectors (left), and the MSLN activator on a first vector and HLA-A*02 blocker on a second vector (right).

FIG. 7 is a diagram illustrating a dual vector system encoding a split activator CAR with a Fab and an inhibitory (blocker) scFv CAR receptor. One vector contains (construct 1, top) encodes the scFv CAR blocker and the VH-CH1 domain, hinge, transmembrane and intracellular domains of the activator CAR. The second vector contains the light chain (LC) of an activator CAR (construct 2, bottom). The second vector can accommodate additional genes in its payload. The two vector system ensures that only cells that express a blocker can express a functional activator by splitting the activator CAR at the Fab domain, and eliminates the need to enrich for cells expressing the blocker receptor post co-transfection or co-transduction.

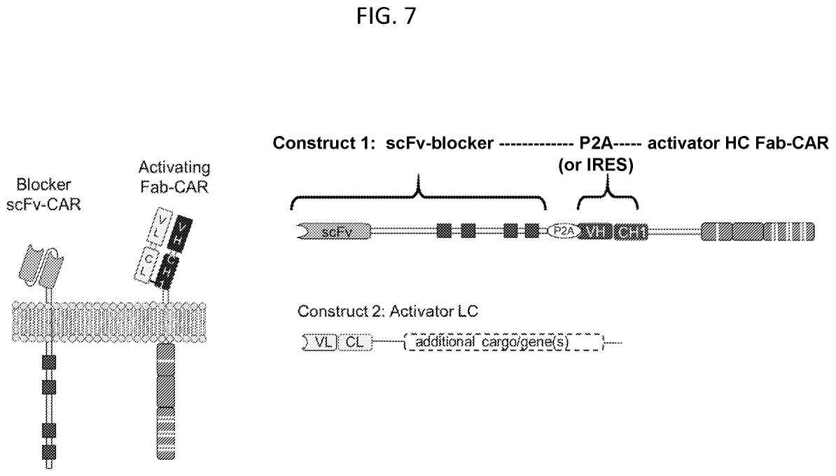

FIG. 8 is a diagram illustrating a dual vector system encoding a split activator CAR and a blocker scFv CAR receptor where the activator CAR is split via the hinge region. One vector (construct 1, top) encodes the scFv blocker CAR and the hinge, transmembrane (TM) and intracellular domains (ICD) of the activator CAR. The second vector (construct 2, bottom) encodes the scFv, hinge and TM domains of the activator CAR. The second vector 2 can thus accommodate additional gene(s) in its payload. In this approach, the same “construct 1” can be used across multiple combinations of activators and blockers utilizing a common blocker receptor. The second vector has a smaller overall insert size, and can thus easily accommodate an additional payload encoding any gene or gene fragment within the size limit of the vector, such as shRNAs, cytokines, kill switch proteins, dominant negative constructs, and the like.

FIG. 9 is a diagram illustrating a dual vector system encoding a split activator CAR and a blocker scFv CAR receptor where the activator CAR is split via the hinge region. One vector (construct 1, top) encodes the scFv blocker CAR and the scFv, hinge and transmembrane (TM) regions of the activator CAR. The second vector (construct 2, bottom) encodes the hinge, TM and intracellular domains (ICD) domains of the activator CAR. The second vector can accommodate additional genes in its payload.

FIG. 10A is a plot (left) and a pair of diagrams (right) showing that cysteine-mediated disulfide formation in the wild type (wt) CD8 hinge facilitate split CAR function. The cysteines in CD8 hinge region were mutated to serines (CD8(S) mutant hinge), and function of the split CAR with and without the mutated hinge was compared.

FIG. 10B are a pair of FACS plots showing expression of the activator receptor as measured by staining with a human CD19-Fc fusion protein and blocking receptor as measured by staining with biotinylated HLA-A*02:01-MAGE-A3 peptide-MHC.

FIG. 11A is a pair of plots (left) and a pair of diagrams (right) showing that split activator CD19 CAR constructs in combination with an HLA-A*02 blocker receptor mediate robust blocking of the CD19 CAR by the HLA-A*02:01 blocker. On the top, full length CD19 activator CAR and full length HLA-A*02 blocker were co-transfected on separate vectors as a control. On the bottom, a first construct encoding the full length HLA-A*02 blocker, followed by a P2A cleavage site and then the activator hinge, TM and ICD (signaling domain, or SD) was co-transfected with a second construct encoding the activator ligand binding domain (LBD), hinge and TM domains.

FIG. 11B is a pair of FACS plots showing expression of CD19 CAR activator and HLA-A*02 blocker receptors in Jurkat effector cells when the cells were transfected with vectors encoding activator and blockers separately (left), and when the cells were transfected with a first vector encoding the HLA-A*02 Blocker, a P2A site, and CD19 CAR hinge-TM-ICD domains, and a second vector encoding the CD19 LBD, hinge and TM domains (right).

FIG. 12A is a plot (top) and a table (bottom) showing that Fos-Jun hinge mediated split CD19 CAR activator receptors show reduced but comparable sensitivity when compared to full length CD19 CAR activator receptors. LBD: ligand binding domain; SD: signaling domain; sCAR: split CAR.

FIG. 12B is a series of diagrams (top) and FACS plots (bottom) showing expression of the FMC63 scFv directed against CD19 in cells expressing, from left to right, (1) the CD19 CAR, (2) a construct with the CD19 CAR ligand binding domain and Fos hinge and transmembrane domain, (3) a construct with the Jun hinge, transmembrane domain, and intracellular domain, and (4) both the construct with the CD19 CAR ligand binding domain, Fos hinge, and TM as well as the construct with the Jun hinge TM, and ICD.

FIG. 13 is a diagram (top) and a plot (bottom) showing that dual vectors containing a split CAR and an accessory kill-switch gene, demonstrating feasibility of using the additional vector real estate for additional cargo with additional functions. rLuc: Renilla luciferase; KS: kill switch; SD: signaling domain; LBD: ligand binding domain. The bottom plot shows that upon activation with tamoxifen, the kill switch gene is able to induce killing of target cells as assessed by Annexin V staining. Cells transformed with the vector encoding the kill switch and P2A cleavage site shows reduced, but inducible, killing upon addition of tamoxifen.

FIG. 14 is a diagram illustrating the design of a dual vector system encoding a split TCR activator and a blocker receptor. One vector (construct 1, top) encodes a scFv-blocker and the alpha subunit of an activator TCR. The blocker could also be an inhibitory TCR, i.e. the TCR alpha and/or beta subunits fused to an inhibitory intracellular domain. The second vector (construct 2, bottom) encodes the beta subunit of the activator TCR. The second vector can accommodate additional genes in its payload.

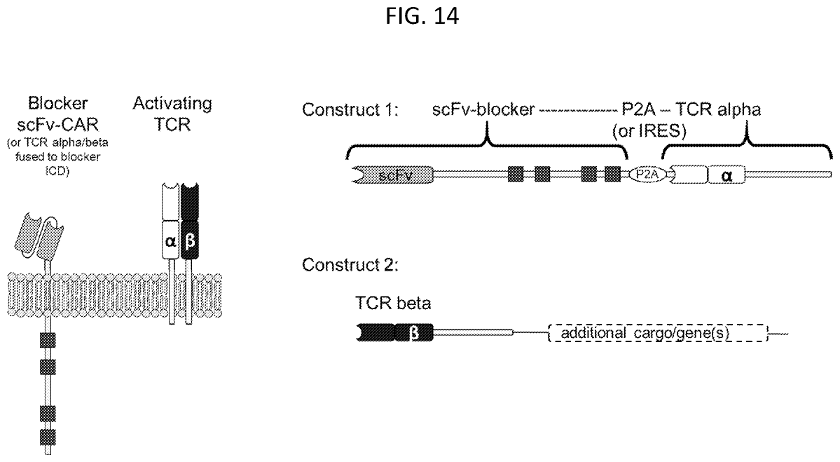

FIG. 15 is a diagram illustrating the design of a dual vector system encoding a split TCR activator and a blocker receptor. One vector (construct 1, top) encodes an scFv-blocker and the beta subunit of an activator TCR. The blocker could also be an inhibitory TCR, i.e. the TCR alpha and/or beta subunits fused to an inhibitory intracellular domain. The second vector (construct 2, bottom) encodes the alpha subunit of the activator TCR. The second vector can accommodate additional genes in its payload.

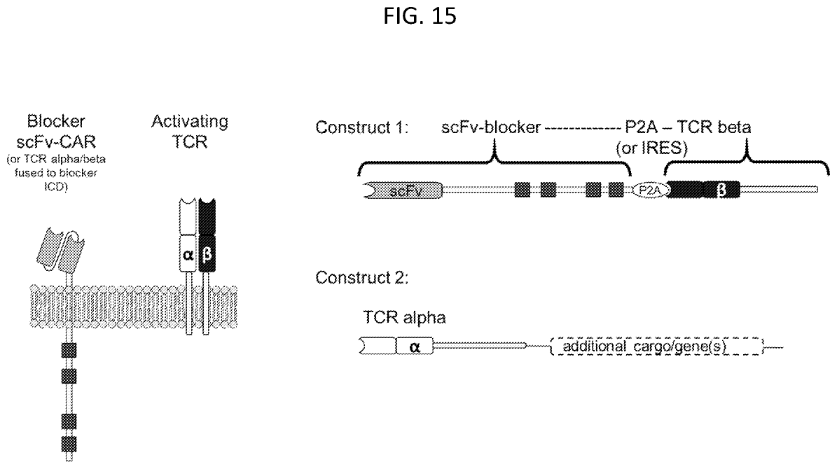

### DETAILED DESCRIPTION

Viral transfer of genes is limited by insert size, and there are situations when one vector cannot accommodate all of the genes required for a therapeutic effector cell. In these cases, two (or more) vectors need to be used to introduce multiple genes into effector cells. However, co-transfecting or co-transducing effector cells with multiple vectors can lead to the heterogeneous uptake of one or both vectors. Successful and safe implementation of some therapies requires that the majority of effector cells carry both transgenes. One example of such a therapy is the two receptor based immune cell therapy described in further detail below. Using this approach, immune cells are transformed with two receptors, an activator and an inhibitory (blocker) receptor. The activator receptor recognizes an antigen expressed by cancer cells, while the antigen of the blocker receptor is not expressed in cancer cells, for example through loss of heterozygosity. Immune cells expressing the two receptors can integrate signals from the two receptors at the cellular level, and thus selectively target tumor cells, but not non-tumor cells. Although blocker-only effectors are often tolerated, minimizing immune cells that uptake and therefore express only the activator receptor may reduce the likelihood that transplanted immune cells will be activated by non-cancer cells that express the activator receptor antigen. The dual vector system described herein, in which the activator receptor is split into two parts, such that both vectors in a single cell are necessary to generate a functional activator, prevents the occurrence of activator-only immune cells. In this system, one vector carries the blocker receptor and one of two parts of the activator receptor, this first part of the activator receptor being non-functional in the absence of a second part. The second vector carries the second part of the activator receptor. Because the activator receptor is encoded by two separate vectors, the total polynucleotide sequence of the vector system is increased compared to the vector system having only one vector. For example and without limitation, the second vector, having only one part of the activator receptor, can carry additional accessory genes, such as markers, reporters, shRNA, or genes that induce apoptosis (kill switch genes). Advantageously, compared to a vector system without the split activator receptor, a vector system as disclosed herein may eliminate heterogeneity in effector cells, because only cells that receive both vectors of the vector system express a functional activator receptor. Thereby, the vector system ensures preferential survival and/or expansion of cells transduced with both vectors compared to cells receiving no vector or only one of the two vectors. Advantageously, the vector systems disclosed herein may be used in cases where more than one vector is required to transfer all the genetic material needed for a cell-based therapeutic.

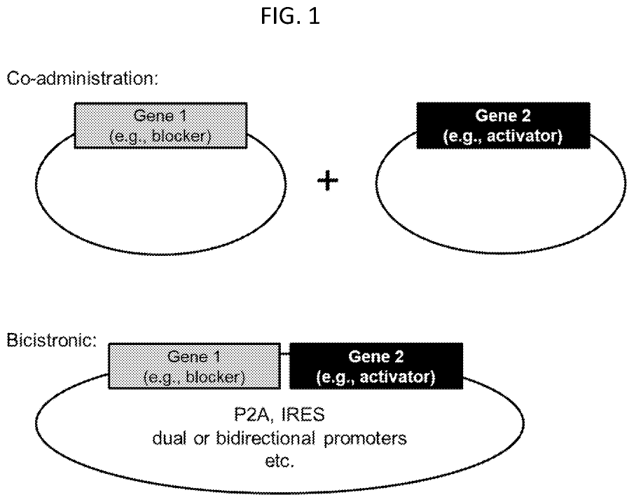

Conventional methods of transforming cells to co-express two genes are shown FIG. 1. In the first method, cells are co-transformed simultaneously with two vectors, each encoding a specific gene of interest. However, in this approach, a subset of cells receive both vectors, while other cells receive only one of the two vectors. Another method is to use a bicistronic vector where the two genes are separated by a self-cleaving peptide, such as P2A, an internal ribosome entry site (IRES), or are driven by dual or bidirectional promoters. A downside of the bicistronic single vector is that the insert size needed for both genes may exceed the size limit of the vector.

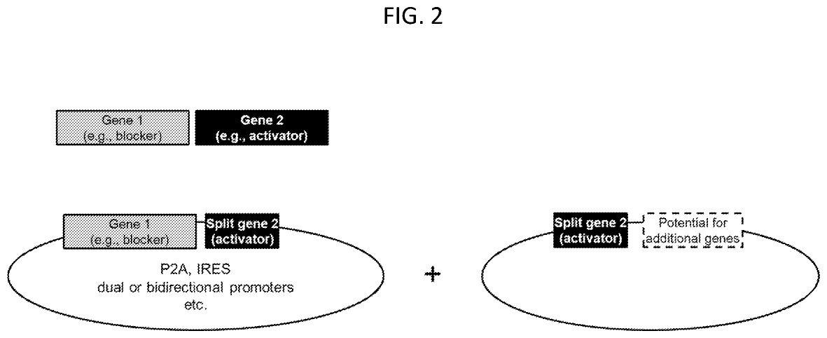

An embodiment of the vector systems disclosed herein is illustrated in in FIG. 2. In this exemplary embodiment, the vector system comprises two vectors. However, one, or potentially both, of the sequences encoding the activator and inhibitory receptors are partitioned between the two vectors. Thus, functional receptors are produced only if both vectors are taken up by a single cell. Another advantage is that, because the insert sequence is split between two vectors, the system can deliver larger payloads, such as larger genes, or even additional sequences, such as sequences encoding additional genes, shRNAs or promoters, to the cell. In the context of the dual receptor system for adoptive cell therapy described herein, the dual vector system can be used to deliver both the activator and blocker receptors. By splitting the activator receptor gene between the two vectors, only cells that have taken up both vectors will express a functional activator receptor. The dual vector system thus can be used to avoid producing cells that only express an activator receptor, and not a blocker receptor, and maximize cells that express both a functional activator receptor and a functional blocker receptor.

Accordingly, the disclosure provides a dual vector system and methods of using the dual vector system to transform cells with two genes.

### Definitions

Prior to setting forth this disclosure in more detail, it may be helpful to an understanding thereof to provide definitions of certain terms to be used herein.

Unless defined otherwise, all technical and scientific terms used herein have the same meaning as commonly understood by those of ordinary skill in the art to which the invention belongs. Although any methods and materials similar or equivalent to those described herein can be used in the practice or testing of particular embodiments, preferred embodiments of compositions, methods and materials are described herein. For the purposes of the present disclosure, the following terms are defined below. Additional definitions are set forth throughout this disclosure.

The articles “a,” “an,” and “the” are used herein to refer to one or to more than one (i.e., to at least one, or to one or more) of the grammatical object of the article. By way of example, “an element” means one element or one or more elements.

The use of the alternative (e.g., “or”) should be understood to mean either one, both, or any combination thereof of the alternatives.

The term “and/or” should be understood to mean either one, or both of the alternatives.

The term “part” refers to a piece or a segment of something such as an object, method or period of time, which combined with other parts makes up the whole. For example, a part of an activator receptor refers to polypeptide sequence comprising some, but not all, of the activator receptor.

The term “split” refers to breaking something into parts. For example, a receptor that is split has been broken into one or more parts. The split receptors of the disclosure can be split in multiple ways, all of which are envisaged as within the scope of the instant disclosure. For example, the receptor can be split at the antigen binding domain, so that a first part of the receptor comprises a first part of the antigen binding domain and the second part of the receptor comprises a second part of the antigen binding domain. Alternatively, the receptor can be split at the hinge and/or transmembrane domain, so that the first part of the receptor comprises the antigen binding domain, as well as hinge and transmembrane domains, and the second part of the receptor comprises the intracellular domain, as well as hinge and transmembrane domains.

As used herein, the term “functional receptor” refers to receptor capable of carrying out one or more activities of the receptor.

As used herein, a “vector system” refers to one or more vectors. The one or more vectors may be designed to work in concert for a particular application, or to produce a desired transformed cell.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are present that materially affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment. Thus, the appearances of the foregoing phrases in various places throughout this specification are not necessarily all referring to the same embodiment. Furthermore, the particular features, structures, or characteristics may be combined in any suitable manner in one or more embodiments. It is also understood that the positive recitation of a feature in one embodiment, serves as a basis for excluding the feature in a particular embodiment.

As used herein, the term “about” or “approximately” refers to a quantity, level, value, number, frequency, percentage, dimension, size, amount, weight or length that varies by as much as 15%, 10%, 9%, 8%, 7%, 6%, 5%, 4%, 3%, 2% or 1% to a reference quantity, level, value, number, frequency, percentage, dimension, size, amount, weight or length. In one embodiment, the term “about” or “approximately” refers a range of quantity, level, value, number, frequency, percentage, dimension, size, amount, weight or length±15%, ±10%, ±9%, ±8%, ±7%, ±6%, ±5%, ±4%, ±3%, ±2%, or ±1% about a reference quantity, level, value, number, frequency, percentage, dimension, size, amount, weight or length.

As used herein, the term “isolated” means material that is substantially or essentially free from components that normally accompany it in its native state. In particular embodiments, the term “obtained” or “derived” is used synonymously with isolated.

The terms “subject,” “patient” and “individual” are used interchangeably herein to refer to a vertebrate, preferably a mammal, more preferably a human. Tissues, cells, and their progeny of a biological entity obtained in vivo or cultured in vitro are also encompassed. A “subject,” “patient” or “individual” as used herein, includes any animal that exhibits pain that can be treated with the vectors, compositions, and methods contemplated herein. Suitable subjects (e.g., patients) include laboratory animals (such as mouse, rat, rabbit, or guinea pig), farm animals, and domestic animals or pets (such as a cat or dog). Non-human primates and, preferably, human patients, are included.

As used herein “treatment” or “treating,” includes any beneficial or desirable effect, and may include even minimal improvement in symptoms. “Treatment” does not necessarily indicate complete eradication or cure of the disease or condition, or associated symptoms thereof.

As used herein, “prevent,” and similar words such as “prevented,” “preventing” etc., indicate an approach for preventing, inhibiting, or reducing the likelihood of a symptom of disease. It also refers to delaying the onset or recurrence of a disease or condition or delaying the occurrence or recurrence of the symptoms of a disease. As used herein, “prevention” and similar words also includes reducing the intensity, effect, symptoms and/or burden of disease prior to onset or recurrence.

As used herein, the term “amount” refers to “an amount effective” or “an effective amount” of a virus to achieve a beneficial or desired prophylactic or therapeutic result, including clinical results.

A “prophylactically effective amount” refers to an amount of a virus effective to achieve the desired prophylactic result. Typically but not necessarily, since a prophylactic dose is used in subjects prior to or at an earlier stage of disease, the prophylactically effective amount is less than the therapeutically effective amount.

A “therapeutically effective amount” of a virus or cell may vary according to factors such as the disease state, age, sex, and weight of the individual, and the ability of the virus or cell to elicit a desired response in the individual. A therapeutically effective amount is also one in which any toxic or detrimental effects of the virus or cell are outweighed by the therapeutically beneficial effects. The term “therapeutically effective amount” includes an amount that is effective to “treat” a subject (e.g., a patient).

An “increased” or “enhanced” amount of a physiological response, e.g., electrophysiological activity or cellular activity, is typically a “statistically significant” amount, and may include an increase that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the level of activity in an untreated cell.

A “decrease” or “reduced” amount of a physiological response, e.g., electrophysiological activity or cellular activity, is typically a “statistically significant” amount, and may include an decrease that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the level of activity in an untreated cell.

By “maintain,” or “preserve,” or “maintenance,” or “no change,” or “no substantial change,” or “no substantial decrease” refers generally to a physiological response that is comparable to a response caused by either vehicle, or a control molecule/composition. A comparable response is one that is not significantly different or measurable different from the reference response.

In general, “sequence identity” or “sequence homology” refers to an exact nucleotide-to-nucleotide or amino acid-to-amino acid correspondence of two polynucleotides or polypeptide sequences, respectively. Typically, techniques for determining sequence identity include determining the nucleotide sequence of a polynucleotide and/or determining the amino acid sequence encoded thereby, and comparing these sequences to a second nucleotide or amino acid sequence. Two or more sequences (polynucleotide or amino acid) can be compared by determining their “percent identity.” The percent identity of two sequences, whether nucleic acid or amino acid sequences, is the number of exact matches between two aligned sequences divided by the length of the shorter sequences and multiplied by 100. Percent identity may also be determined, for example, by comparing sequence information using the advanced BLAST computer program, including version 2.2.9, available from the National Institutes of Health. The BLAST program is based on the alignment method of Karlin and Altschul, Proc. Natl. Acad. Sci. USA 87:2264-2268 (1990) and as discussed in Altschul, et al., J. Mol. Biol. 215:403-410 (1990); Karlin And Altschul, Proc. Natl. Acad. Sci. USA 90:5873-5877 (1993); and Altschul et al., Nucleic Acids Res. 25:3389-3402 (1997). Briefly, the BLAST program defines identity as the number of identical aligned symbols (generally nucleotides or amino acids), divided by the total number of symbols in the shorter of the two sequences. The program may be used to determine percent identity over the entire length of the proteins being compared. Default parameters are provided to optimize searches with short query sequences in, for example, with the blastp program. The program also allows use of an SEG filter to mask-off segments of the query sequences as determined by the SEG program of Wootton and Federhen, Computers and Chemistry 17:149-163 (1993). Ranges of desired degrees of sequence identity are approximately 80% to 100% and integer values therebetween. Typically, the percent identities between a disclosed sequence and a claimed sequence are at least 80%, at least 85%, at least 90%, at least 95%, or at least 98%.

The term “exogenous” is used herein to refer to any molecule, including nucleic acids, protein or peptides, small molecular compounds, and the like that originate from outside the organism. In contrast, the term “endogenous” refers to any molecule that originates from inside the organism (i.e., naturally produced by the organism).

The term “MOI” is used herein to refer to multiplicity of infection, which is the ratio of agents (e.g. viral particles) to infection targets (e.g. cells).

In the present description, any concentration range, percentage range, ratio range, or integer range is to be understood to include the value of any integer within the recited range and, when appropriate, fractions thereof (such as one tenth and one hundredth of an integer), unless otherwise indicated. The term “about”, when immediately preceding a number or numeral, means that the number or numeral ranges plus or minus 10%.

As used herein, a “target cell” refers to cell that is targeted by an adoptive cell therapy. For example, a target cell can be cancer cell, which can be killed by the transplanted T cells of the adoptive cell therapy. Target cells of the disclosure express an activator ligand as described herein, and do not express an inhibitor ligand.

All publications and patents mentioned herein are hereby incorporated by reference in their entirety as if each individual publication or patent was specifically and individually indicated to be incorporated by reference. In case of conflict, the present application, including any definitions herein, will control. However, mention of any reference, article, publication, patent, patent publication, and patent application cited herein is not, and should not be taken as an acknowledgment, or any form of suggestion, that they constitute valid prior art or form part of the common general knowledge in any country in the world.

### EMBODIMENTS

The disclosure provides a vector system comprising two vectors, wherein the first vector comprises a polynucleotide comprising a sequence encoding a first part of an activator receptor, and the second vector comprises a polynucleotide comprising a sequence encoding a second part of the activator receptor. The activator receptor, for example a chimeric antigen receptor (CAR) or T cell receptor (TCR), comprises an antigen binding domains. The activator receptor can split between the two vectors via the antigen binding domain. When the activator receptor is split via the antigen binding domain, the first vector comprises a polynucleotide comprising a sequence encoding a first part of the activator antigen binding domain, and the second vector comprises a polynucleotide comprising a sequence encoding a second part of the activator antigen binding domain. When a cell is transduced with the first and second vectors, the first and second parts of the antigen associate in the cell to form a functional activator receptor.

Any suitable methods of splitting the activator receptor via the antigen binding domain are envisaged as within the scope of the instant disclosure. For example, the antigen binding domain may be a Fab (also referred to herein as a Fab fragment). Fab (fragment antigen-binding) fragments are the antibody binding regions of an antibody. The Fab fragment contains one complete antibody light chain, including the variable (VL) and constant (CL) domains, and the variable (VH) and constant 1 (CH1) portions of one antibody heavy chain. The Fab can be further divided into a variable fragment (Fv) composed of the VH and VL domains, and a constant fragment composed of the CL and CH1 domains. In general, Fab fragment comprise about 440-450 amino acids and have a molecular weight of approximately 55 kiloDaltons (kDa).

In some embodiments, the activator receptor antigen binding domain comprises a Fab. In some embodiments, the first part of the activator receptor comprises a light chain of the Fab, and the second part of the activator receptor comprises a heavy chain of the Fab, the transmembrane domain and the intracellular domain. In some embodiments, the first part of the activator receptor comprises a heavy chain of the Fab, and the second part of the activator receptor comprises a light chain of the Fab, the transmembrane domain and the intracellular domain. In some embodiments, the receptor comprises a CAR. In some embodiments, the complete CAR (i.e., as produced by the combination of both vectors) comprises an extracellular antigen binding domain, a hinge domain, a transmembrane domain, and an intracellular domain. In some embodiments, the first part of the activator receptor comprises a light chain of a Fab, and the second part of the activator comprises a heavy chain of a Fab, a hinge domain, a transmembrane domain, and an intracellular domain. In some embodiments, the first part of the activator receptor comprises a heavy chain of a Fab, and the second part of the activator comprises a light chain of a Fab, a hinge domain, a transmembrane domain, and an intracellular domain.

In some embodiments, the first part of the activator receptor comprises a light chain of a Fab, a hinge domain, a transmembrane domain, and an intracellular domain and the second part of the activator comprises a heavy chain of a Fab. In some embodiments, the first part of the activator receptor comprises a heavy chain of a Fab, a hinge domain, a transmembrane domain, and an intracellular domain and the second part of the activator comprises a light chain of a Fab.

In some embodiments, extracellular domain of the activator receptor comprises a Fab. In some embodiments, the light chain of the Fab comprises a light chain variable domain (VL) and a light chain constant domain (CL). Any suitable variable domain is envisaged as within the scope of the instant disclosure. The skilled artisan will be able to select an appropriate VL, and the corresponding VH, for a Fab with a desired antigen specificity from antibody sequences known in the art. In some embodiments, the CL domain of the Fab comprises a sequence of RTVAAPSVFIFPPSDEQLKSGTASVVCLLNNFYPREAKVQWKVDNALQSGNSQES VTEQDSKDSTYSLSSTLTLSKADYEKHKVYACEVTHQGLSSPVTKSFNRGEC, or a sequence having at least 90%, at least 95%, at least 97% or at least 99% identity thereto. In some embodiments, the CL domain of the Fab comprises a sequence of RTVAAPSVFIFPPSDEQLKSGTASVVCLLNNFYPREAKVQWKVDNALQSGNSQES VTEQDSKDSTYSLSSTLTLSKADYEKHKVYACEVTHQGLSSPVTKSFNRGEC. In some embodiments, the heavy chain of the Fab comprises a heavy chain variable domain (VH), and a heavy chain constant domain (CH), or a portion thereof. In some embodiments, the CH domain of the Fab comprises a sequence of WGQGTSVTVSSASTKGPSVFPLAPSSKSTSGGTAALGCLVKDYFPEPVTVSWNSG ALTSGVHTFPAVLQSSGLYSLSSVVTVPSSSLGTQTYICNVNHKPSNTKVDKKVEP KSCTTTPAPRPPTPAPTIASQPLSLRPEACRPAAGGAVHTRGLDFACD, or a sequence having at least 90%, at least 95%, at least 97% or at least 99% identity thereto. In some embodiments, the CH domain of the Fab comprises a sequence of WGQGTSVTVSSASTKGPSVFPLAPSSKSTSGGTAALGCLVKDYFPEPVTVSWNSG ALTSGVHTFPAVLQSSGLYSLSSVVTVPSSSLGTQTYICNVNHKPSNTKVDKKVEP KSCTTTPAPRPPTPAPTIASQPLSLRPEACRPAAGGAVHTRGLDFACD. In some embodiments, the CH domain of the Fab comprises a sequence of WGQGTLVTVSSASTKGPSVFPLAPSSKSTSGGTAALGCLVKDYFPEPVTVSWNSG ALTSGVHTFPAVLQSSGLYSLSSVVTVPSSSLGTQTYICNVNHKPSNTKVDKKVEP KSCTTTPAPRPPTPAPTIASQPLSLRPEACRPAAGGAVHTRGLDFACD, or a sequence having at least 90%, at least 95%, at least 97% or at least 99% identity thereto. In some embodiments, the CH domain of the Fab comprises a sequence of

WGQGTLVTVSSASTKGPSVFPLAPSSKSTSGGTAALGCLVKDYFPEPVTV
SWNSGALTSGVHTFPAVLQSSGLYSLSSVVTVPSSSLGTQTYICNVNHKP
SNTKVDKKVEPKSCTTTPAPRPPTPAPTIASQPLSLRPEACRPAAGGAVH
TRGLDFACD.

Additional split antigen binding domains are envisaged as within the scope of the instant disclosure. For example, antigen binding domains comprising larger fragments of the heavy chain constant region may be suitable for use in the activator receptors described herein. Antigen binding domains that comprise the constant 3 (CH3) domain of the heavy chain (CH3), for example, can be engineered with knob into hole technology to promote specific heavy chain to heavy chain interactions. Thus, if one vector comprises a polynucleotide sequence encoding a first heavy chain, for example a heavy chain with “knob” mutations in CH3, which is fused to a hinge domain, transmembrane domain, and intracellular domain of a CAR, and the second vector comprises a polynucleotide sequence encoding a second heavy chain, for example a heavy chain with “hole” mutations in CH3, the interaction of the heavy chains can produce a functional receptor. The skilled artisan will appreciate that the reciprocal arrangement of elements will also produce a functional receptor, i.e., the hole heavy chain can be fused to the hinge domain, transmembrane domain, and intracellular domains of the activator, while the knob heavy chain is not so disposed.

Knob into hole technology is described, for example, in Liu et al. (2017) Front. Immunol., the contents of which are incorporated herein by reference. In knob into hole, CH3 domains are engineered to create either a “knob” or a “hole” in each heavy chain to promote heterodimerization. This is based on the principle that heavy chains (H) of human immunoglobulin IgG interact directly through their CH3 domains. The knob mutations, for example tyrosine (Y), mutate residues to large volume amino acids, while the reciprocal hole mutations at are positioned at an equivalent position, and mutate an amino acid residue to a small volume amino acid, for example threonine (T) to create a hole into which the knob mutation fits.

One advantage of splitting the CAR through the antigen binding domain is that any suitable hinge, transmembrane and intracellular domains may be used. In some embodiments, the transmembrane comprises CD8α, CD4, CD28, CD3ζ, or immunoglobulin G (IgG) transmembrane domain. In some embodiments, the hinge comprises an CD8α, CD4, CD28, CD3ζ or IgG hinge. In some embodiments, the intracellular domain comprises a CD3ζ intracellular domain. In some embodiments, the intracellular domain of the activator receptor comprises one or more co-stimulatory domains, for example a co-stimulatory domain isolated or derived from CD27 molecule (CD27), CD28, CD137, TNF receptor superfamily member 4 (OX40), TNF receptor superfamily member 8 (CD30), CD40 molecule (CD40), CD40 ligand (CD40L), CD3ζ, integrin subunit beta 2 (LFA-1), inducible T cell costimulator (ICOS), CD2 molecule (CD2), CD7 molecule (CD7), TNF superfamily member 14 (LIGHT), killer cell lectin like receptor C2 (NKG2C), CD276 molecule (B7-H3), or hematopoietic cell signal transducer (DAP10).

In exemplary embodiments, the hinge, transmembrane of the CAR comprise a CD28 hinge, a CD28 transmembrane domain, and intracellular domain comprises a 4-1BB co-stimulatory domain and CD3 ζ intracellular domain. In some embodiments, the hinge, transmembrane and intracellular domain of the CAR comprise a sequence of FWVLVVVGGVLACYSLLVTVAFIIFWVRSKRSRLLHSDYMNMTPRRPGPTRKHY QPYAPPRDFAAYRSKRGRKKLLYIFKQPFMRPVQTTQEEDGCSCRFPEEEEGGCEL RVKFSRSADAPAYKQGQNQLYNELNLGRREEYDVLDKRRGRDPEMGGKPRRKN PQEGLYNELQKDKMAEAYSEIGMKGERRRGKGHDGLYQGLSTATKDTYDALHM QALPPR, or a sequence having at least 90%, at least 95%, at least 97% or at least 99% identity thereto.

In some embodiments, the activator receptor is a TCR, and the first part of the activator receptor comprises a TCRα chain, and the second part of the activator receptor comprises a TCRβ chain. In some embodiments, the first vector comprises a polynucleotide comprising a sequence encoding a TCRα chain, and the second vector comprises a polynucleotide comprising a sequence encoding a TCRβ chain.

The disclosure provides a vector system comprising two vectors, wherein the first vector comprises a polynucleotide comprising a sequence encoding a first part of an activator receptor, and the second vector comprises a polynucleotide comprising a sequence encoding a second part of the activator receptor. In some embodiments, both the first and second parts of the activator receptor comprise hinge and transmembrane domains, and interactions through the hinge domain, or the hinge and transmembrane domains, bring together the first and second parts of the activator receptor to form a functional receptor in a cell that expresses both parts of the receptor.

In some embodiments, the activator receptor is a CAR.

In some embodiments, the first part of the activator receptor comprises a first hinge domain, a first transmembrane domain and the intracellular domain, and the second part of the activator comprises the antigen binding domain, a second hinge domain and a second transmembrane domain. In some embodiments, the first part of the activator receptor comprises the antigen binding domain, a first hinge domain and a first transmembrane domain, and the second part of the activator comprises a second hinge domain, a second transmembrane domain and the intracellular domain. In some embodiments, the first and second hinge domains form a dimer, thereby producing a functional receptor when expressed in a cell. In some embodiments, the first and second hinge domains and transmembrane domains form a dimer, thereby producing a functional receptor when expressed in a cell.

Any suitable hinge domain, transmembrane domain, or combination thereof that induces dimerization, either homodimerization or heterodimerization, is contemplated as within the scope of the instant disclosure. Exemplary domains that induce dimerization include, but are not limited to, leucine zippers derived from the bZIP (basic leucine zipper) family of transcription factors. The leucine zipper is a three-dimensional structural motif, which has periodic repetition of leucine residues at every seventh position over a distance covering approximately eight helical turns. The leucine zipper is created by the dimerization of two specific alpha helix monomers bound to DNA. The leucine zipper is formed by amphipathic interaction between two ZIP domains. The ZIP domain is found in the alpha-helix of each monomer, and contains leucines, or leucine-like amino acids. These amino acids are spaced along polypeptide such that when the sequence is coiled in alpha-helix, the leucine residues line up on the same side of the helix, forming the ZIP domain. The leucines from two ZIP domains interact, reversibly forming dimers (the zipper).

In some embodiments, the first and second hinge domains form a homodimer. In some embodiments, the first and second hinge domains comprise CD8α hinge domains. CD8α proteins form both CD8α homodimers, and CD8α/CD8β heterodimers. Without wishing to be bound by theory, and as described in further detail in Example 2 below, it is thought that the CD8α homodimerization is mediated by cysteine residues in the CD8α hinge domain. In some embodiments, the first and second CD8α hinge domains comprise a sequence of TTTPAPRPPTPAPTIASQPLSLRPEACRPAAGGAVHTRGLDFACD, or a sequence having at least 90%, at least 95%, at least 97% or at least 99% identity thereto. In some embodiments, the first and second CD8α hinge domains comprise cysteines (C) at positions 27 and 44 of TTTPAPRPPTPAPTIASQPLSLRPEACRPAAGGAVHTRGLDFACD. In some embodiments, the first and second CD8α hinge domains comprises a sequence of TTTPAPRPPTPAPTIASQPLSLRPEACRPAAGGAVHTRGLDFACD. In some embodiments, association of the CD8α hinges of the first and second parts of the activator receptor in the cell produces a functional activator receptor.

Any suitable hinge domain, transmembrane domain, or combination thereof that induces dimerization, either homodimerization or heterodimerization, is contemplated as within the scope of the instant disclosure. Exemplary domains that induce dimerization include, but are not limited to, leucine zippers derived from the bZIP (basic leucine zipper) family of transcription factors. The leucine zipper is a three-dimensional structural motif, which has periodic repetition of leucine residues at every seventh position over a distance covering approximately eight helical turns. The leucine zipper is created by the dimerization of two specific alpha helix monomers bound to DNA. The leucine zipper is formed by amphipathic interaction between two ZIP domains. The ZIP domain is found in the alpha-helix of each monomer, and contains leucines, or leucine-like amino acids. These amino acids are spaced along polypeptide such that when the sequence is coiled in alpha-helix, the leucine residues line up on the same side of the helix, forming the ZIP domain. The leucines from two ZIP domains interact, reversibly forming dimers (the zipper).

In some embodiments, the first and second hinge domains form a heterodimer. In some embodiments, the heterodimer is a Fos/Jun heterodimer. Fos proto-oncogene, AP-1 transcription factor subunit (Fos) and Jun proto-oncogene, AP-1 transcription factor subunit (Jun) family proteins function as dimeric, bZIP family transcription factors that bind to AP-1 regulatory elements in the promoter and enhancer regions of numerous mammalian genes. Jun proteins form both homodimers and heterodimers with Fos proteins. Dimerization is mediated through a leucine zipper formed by the Fos and Jun ZIP domains.

In some embodiments, the first part of the activator comprises a first hinge domain, and the second part of the activator comprises a second hinge domain. The first hinge domain can be a Fos hinge domain, i.e. it comprises a sequence isolated or derived from a Fos family protein such as human Fos, and the second hinge domain can be a Jun hinge domain, i.e. it comprises a sequence isolated or derived from a Jun family protein such as human Jun. The person of ordinary skill in the art will appreciate that the reciprocal arrangement is also within the scope of the instant disclosure, and that the first hinge domain can comprise a Jun sequence and the second hinge domain can comprise a Fos sequence. In some embodiments, the Fos and Jun hinge domains associate, thereby forming a functional receptor in a cell.

In some embodiments, the Fos hinge domain comprises a sequence of SDGSLTDTLQAETDQLEDEKSALQTEIANLLKEKEKLEFILAAHGS, or a sequence having at least 90%, at least 95%, or at least 97% identity thereto. In some embodiments, the Fos hinge domain comprises a sequence of SDGSLTDTLQAETDQLEDEKSALQTEIANLLKEKEKLEFILAAHGS. In some embodiments, the Fos hinge domain further comprises an additional hinge sequence. For example, the Fos hinge can comprise both the Fos sequence that promotes heterodimerization of the activator receptor, and additional hinge sequences that promote one or more additional functions of the activator receptor. In some embodiments, the additional hinge sequence comprises a CD8α hinge sequence. In some embodiments, the CD8α hinge sequence comprises TTTPAPRPPTPAPTIASQPLSLRPEASRPAAGGAVHTRGLDFASD, or a sequence having at least 90%, at least 95%, or at least 97% identity thereto. In some embodiments, the CD8α hinge sequence does not comprise cysteines at positions 27 and 44 of TTTPAPRPPTPAPTIASQPLSLRPEASRPAAGGAVHTRGLDFASD. In some embodiments, the CD8α hinge sequence comprises serines at positions 27 and 44 of TTTPAPRPPTPAPTIASQPLSLRPEASRPAAGGAVHTRGLDFASD. In some embodiments, the CD8α hinge sequence comprises TTTPAPRPPTPAPTIASQPLSLRPEASRPAAGGAVHTRGLDFASD. In some embodiments, the Fos hinge comprises a sequence of TTTPAPRPPTPAPTIASQPLSLRPEASRPAAGGAVHTRGLDFASDGSLTDTLQAETD QLEDEKSALQTEIANLLKEKEKLEFILAAHGS, or a sequence having at least 90%, at least 95%, or at least 97% identity thereto. In some embodiments, the Fos hinge comprises a sequence of

TTTPAPRPPTPAPTIASQPLSLRPEASRPAAGGAVHTRGLDFASDGSLTD
TLQAETDQLEDEKSALQTEIANLLKEKEKLEFILAAHGS.

In some embodiments, the Jun hinge domain comprises a sequence of SDGSRIARLEEKVKTLKAQNSELASTANMLREQVAQLKQKVMNHG, or a sequence having at least 90%, at least 95%, or at least 97% identity thereto. In some embodiments, the Jun hinge domain comprises a sequence of SDGSRIARLEEKVKTLKAQNSELASTANMLREQVAQLKQKVMNHG. In some embodiments, the Jun hinge domain further comprises an additional hinge sequence. For example, the Fos hinge can comprise both the Jun sequence that promotes heterodimerization of the activator receptor, and additional hinge sequences that promote one or more additional functions of the activator receptor. In some embodiments, the additional hinge sequence comprises a CD8α hinge sequence. In some embodiments, the CD8α hinge sequence comprises TTTPAPRPPTPAPTIASQPLSLRPEASRPAAGGAVHTRGLDFASD, or a sequence having at least 90%, at least 95%, or at least 97% identity thereto. In some embodiments, the CD8α hinge sequence does not comprise cysteines at positions 27 and 44 of TTTPAPRPPTPAPTIASQPLSLRPEASRPAAGGAVHTRGLDFASD. In some embodiments, the CD8α hinge sequence comprises serines at positions 27 and 44 of TTTPAPRPPTPAPTIASQPLSLRPEASRPAAGGAVHTRGLDFASD. In some embodiments, the CD8α hinge sequence comprises TTTPAPRPPTPAPTIASQPLSLRPEASRPAAGGAVHTRGLDFASD. In some embodiments, the Jun hinge comprises a sequence of TTTPAPRPPTPAPTIASQPLSLRPEASRPAAGGAVHTRGLDFASDGSRIARLEEKVK TLKAQNSELASTANMLREQVAQLKQKVMNHG, or a sequence having at least 90%, at least 95%, or at least 97% identity thereto. In some embodiments, the Jun hinge comprises a sequence of

TTTPAPRPPTPAPTIASQPLSLRPEASRPAAGGAVHTRGLDFASDGSRIA
RLEEKVKTLKAQNSELASTANMLREQVAQLKQKVMNHG.

Split activator receptors comprising a CD8α hinge, or a Fos/Jun hinge, can include any suitable antigen binding domain, transmembrane domain, and/or intracellular domain known in the art. Suitable antigen binding domains include, but are not limited to, a single chain variable fragment (scFv), a single chain Fab (scFab), a single domain antibody (sdAb), a fragment antigen binding (Fab), a F(ab′)₂, or a Fab′. Suitable transmembrane domains include, but are not limited to, transmembrane domains isolated or derived from CD8α, CD4, CD28, CD3ζ, immunoglobulin G (IgG), or human leukocyte antigen A (HLA-A). Suitable intracellular domains comprise CD3ζ, CD27, CD28, CD137, OX40, CD30, CD40, CD40L, LFA-1, ICOS, CD2, CD7, LIGHT, NKG2C, B7-H3, or DAP10 intracellular domains, or combinations thereof.

The skilled artisan will appreciate that split activator receptors can, in some cases comprise extracellular sequences on either the first or second portion of the receptor, in addition to the antigen binding domain. Such sequences will not interfere with the function of the activator receptor, and depending on the sequence, may enhance one or more functions of the activator receptor.

In some embodiments, the transmembrane of the first and/or second portion of the activator receptor comprises an HLA-A transmembrane binding domain. In some embodiments, the HLA-A transmembrane domain comprises a sequence of VGIIAGLVLFGAVITGAVVAAVMWRSKRSR, or a sequence having at least 90%, at least 95%, or at least 97% identity thereto. In some embodiments, the HLA-A transmembrane domain comprises a sequence of

VGIIAGLVLFGAVITGAVVAAVMWRSKRSR.

In some embodiments, the activator receptor comprises a CAR, and the CAR comprises a CD28, 4-1BB and CD3ζ sequences. In some embodiments, the CAR comprises a sequence

LLHSDYMNMTPRRPGPTRKHYQPYAPPRDFAAYRSKRGRKKLLYIFKQPF MRPVQTTQEEDGCSCRFPEEEEGGCELRVKFSRSADAPAYKQGQNQLYNELNLG RREEYDVLDKRRGRDPEMGGKPRRKNPQEGLYNELQKDKMAEAYSEIGMKGER RRGKGHDGLYQGLSTATKDTYDALHMQALPPR, or a sequence having at least 90%, at least 95%, or at least 97% identity thereto.

The disclosure provides a dual vector system, wherein one of the two vectors comprises a sequence encoding a blocker receptor and a sequence encoding a portion of an activator receptor.

Methods of encoding multiple polypeptides using a single vector will be known to persons of ordinary skill in the art, and include, inter alia, encoding multiple polypeptides under control of different promoters, or, if a single promoter is used to control transcription of multiple polypeptides, use of sequences encoding internal ribosome entry sites (IRES), ribozymes, protease cleavage site, or self-cleaving peptides.

In some embodiments, one of the two vectors comprises a sequence encoding a blocker receptor and a sequence encoding a portion of an activator receptor, and a sequence encoding an IRES, ribozyme, protease cleavage site, or self-cleaving peptide between the sequence encoding the blocker receptor and the sequence encoding the portion of the activator receptor.

A vector including sequences encoding a blocker receptor and a first part of an activator receptor may include a self-cleaving polypeptide between the two sequences. In some embodiments, the self-cleaving peptide is a viral 2A peptide or has the sequence thereof. Exemplary viral 2A peptides can be isolated or derived from porcine teschovirus-1 (P2A), foot-and-mouth disease virus (F2A), Thosea asigna virus (T2A), and equine rhinitis A virus (E2A), among others.

Without wishing to be bound by theory, 2A peptides are thought to generate two separate polypeptide chains by ribosome skipping, thereby producing equimolar levels of multiple genes from the same mRNA. Ribosome skipping involves the ribosome skipping the synthesis of a peptide bond at the C-terminus of a 2A element, resulting in the separation between the end of the 2A element and the following peptide immediately downstream. The juncture point often occurs between the glycine and proline residues found on the C-terminus.

Exemplary self-cleaving peptides include T2A, P2A, E2A and F2A self-cleaving peptides. In some embodiments, the T2A self-cleaving peptide comprises a sequence of EGRGSLLTCGDVEENPGP. In some embodiments, the P2A self-cleaving peptide comprises a sequence of ATNFSLLKQAGDVEENPGP. In some embodiments, the E2A self-cleaving peptide comprises a sequence of QCTNYALLKLAGDVESNPGP. In some embodiments, the F2A self-cleaving peptide comprises a sequence of

VKQTLNFDLLKLAGDVESNPGP.

Any of T2A, P2A, E2A and F2A self-cleaving peptides can further comprise a linker, such as an N terminal GSG linker. Accordingly, In some embodiments, the P2A self-cleaving peptide comprises a sequence of GSGATNFSLLKQAGDVEENPGP, the T2A self-cleaving peptide comprises a sequence of GSGEGRGSLLTCGDVEENPGP, the E2A self-cleaving peptide comprises a sequence of GSGQCTNYALLKLAGDVESNPGP, and the F2A self-cleaving peptide comprises a sequence of

GSGVKQTLNFDLLKLAGDVESNPGP.

In some embodiments, the first vector comprises a sequence encoding a P2A self-cleaving polypeptide between the sequence encoding the inhibitory receptor and the sequence encoding the first part of the activator. In some embodiments, the he P2A self-cleaving peptide comprises a sequence of GSGATNFSLLKQAGDVEENPGP.

Alternatively, a sequence cleaved by a protease could be used to disjoin the polypeptide sequences encoding the blocker receptor and the portion of an activator receptor. The proteolytically cleavable sequence can include a protease recognition sequence recognized by a protease selected from the group consisting of alanine carboxypeptidase, Armillaria mellea astacin, bacterial leucyl aminopeptidase, cancer procoagulant, cathepsin B, clostripain, cytosol alanyl aminopeptidase, elastase, endoproteinase Arg-C, enterokinase, gastricsin, gelatinase, Gly-X carboxypeptidase, glycyl endopeptidase, human rhinovirus 3C protease, hypodermin C, IgA-specific serine endopeptidase, leucyl aminopeptidase, leucyl endopeptidase, lysC, lysosomal pro-X carboxypeptidase, lysyl aminopeptidase, methionyl aminopeptidase, myxobacter, nardilysin, pancreatic endopeptidase E, picomain 2A, picomain 3C, proendopeptidase, prolyl aminopeptidase, proprotein convertase I, proprotein convertase II, russellysin, saccharopepsin, semenogelase, T-plasminogen activator, thrombin, tissue kallikrein, tobacco etch virus (TEV), togavirin, tiyptophanyl aminopeptidase, U-plasminogen activator, V8, venombin A, venombin AB, and Xaa-pro aminopeptidase. In some cases, the proteolytically cleavable linker can include a protease recognition sequence recognized by a host enzyme, e.g., an enzyme naturally produced by the host cell.

For example, the a protease cleavage site can comprise a matrix metalloproteinase cleavage site, e.g., a cleavage site for a MMP selected from collagenase-1, -2, and -3 (MMP-1, -8, and -13), gelatinase A and B (MMP-2 and -9), stromelysin 1, 2, and 3 (MMP-3, -10, and -11), matrilysin (MMP-7), and membrane metalloproteinases (MT1-MMP and MT2-MMP). For example, the cleavage sequence of MMP-9 is Pro-X-X-Hy (wherein, X represents an arbitrary residue; Hy, a hydrophobic residue) as described in WO 2015/195531. Another example of a protease cleavage site is a plasminogen activator cleavage site, e.g., a uPA or a tissue plasminogen activator (tPA) cleavage site. Specific examples of cleavage sequences of uPA and tPA include sequences comprising Val-Gly-Arg. Another example of a protease cleavage site that can be included in a proteolytically cleavable linker is a tobacco etch virus (TEV) protease cleavage site, e.g., ENLYTQS, where the protease cleaves between the glutamine and the serine. Still further examples of protease cleavages sites in an enterokinase cleavage site, e.g., DDDDK, a thrombin cleavage site (LVPR or CGLVPAGSGP), a furin cleavage site (Arg-X-(Arg/Lys)-Arg, where X is any amino acid), a PreScission protease cleavage site (LEVLFQGP), SLLKSRMVPNFN or SLLIARRMPNFN, cleaved by cathepsin B; SKLVQASASGVN or SSYLKASDAPDN, cleaved by an Epstein-Barr virus protease; RPKPQQFFGLMN, cleaved by MMP-3 (stromelysin); SLRPLALWRSFN, cleaved by MMP-7 (matrilysin); SPQGIAGQRNFN, cleaved by MMP-9; DVDERDVRGFASFL cleaved by a thermolysin-like MMP; SLPLGLWAPNFN, cleaved by matrix metalloproteinase 2 (MMP-2); SLLIFRSWANFN, cleaved by cathespin L; SGWIATVIVIT, cleaved by cathepsin D; SLGPQGIWGQFN, cleaved by matrix metalloproteinase 1 (MMP-l); KKSPGRWGGSV, cleaved by urokinase-type plasminogen activator; PQGLLGAPGILG, cleaved by membrane type 1 matrixmetalloproteinase (MT-MMP); HGPEGLRVGFYESDVMGRGHARLVHVEEPHT, cleaved by stromelysin 3 (or MMP-11), thermolysin, fibroblast collagenase and stromelysin-1; GPQGLAGQRGIV, cleaved by matrix metalloproteinase 13 (collagenase-3); GGSGQRGRKALE, cleaved by tissue-type plasminogen (tPA); SLSALLSSDIFN cleaved by human prostate-specific antigen; SLPRFKIIGGFN, cleaved by kallikrein (hK3); SLLGIAVPGNFN, cleaved by neutrophil elastase; and FFKNIVTPRTPP, cleaved by calpain (calcium activated neutral protease). Depending upon the type of cell expressing the first and second vectors, the person of ordinary skill in the art will be able to select a suitable protease cleavage site, for example a protease cleavage site corresponding to a protease expressed by the cell.

In some embodiments, the vector comprising a sequence encoding a blocker receptor and a sequence encoding a portion of an activator comprises an internal ribosome entry site (IRES). IRES sequences can be isolated or derived from a number of viruses and eukaryotic genes, including, but not limited to Poliovirus, Rhinovirus, Picomaviruses, Hepatitis, and genes such as Fibroblast growth factor (FGF-1 IRES and FGF-2 IRES), Platelet-derived growth factor B (PDGF/c-sis IRES), Vascular endothelial growth factor (VEGF IRES), and Insulin-like growth factor 2 (IGF-II IRES). In some embodiments, the IRES is located between the sequence encoding the blocker receptor and the sequence encoding the portion of the activator receptor. Nucleic acid vector directing the expression of more than one protein from a single vector is known in the art as a multicistronic vector. When an IRES segment is located between two open reading frames in a eukaryotic mRNA (a bicistronic mRNA), it can drive translation of the downstream protein coding region independently of the 5′-cap structure bound to the 5′ end of the mRNA molecule. A single mRNA transcript is generated containing sequences of the first cistron, IRESs, and other downstream cistrons, rather than separate mRNA transcripts as in the conventional approach. During translation, the first cistron is translated by the ribosomal scanning mechanism because it is most proximal to the 5′ cap while the second cistron (and other downstream cistrons) are translated by internal ribosome binding to the IRES.

As used herein, “IRES activity” refers to cap-independent translation initiated by internal ribosome binding, as opposed to cap-dependent translation. “Cap-dependent translation” refers to the mechanism of translation in which the ribosomal unit essential for initiating translation binds to mRNA at or near the 5′ cap region on the mRNA. Cap-dependent translation is purported to proceed by a “ribosome scanning” mechanism whereby the ribosome complex scans the mRNA from the 5′ cap until it encounters an AUG initiation codon. “Cap-independent translation” refers to the mechanism of translation in which the ribosomal unit essential for initiating translation binds to a site on the mRNA without requiring the 5′ cap region. As used herein, the “IRES” is a nucleotide sequence that provides a site for ribosomal binding for cap-independent translation.

Ribozymes may also be used to separate the mRNA sequences encoding the two polypeptides. Ribozymes are RNA molecules that catalyze a variety of chemical reactions such as self-cleavage or ligation. Various naturally occurring ribozymes have been identified in viruses, viroids, and protozoans. One of the first catalytic RNAs was discovered in the satellite RNA of the tobacco ring spot viroid (sTRSV). In vivo, this pathogenic viroid was shown to act in cis and self-cleave during replication. Since the discovery of the first ribozyme, various classes of natural ribozymes, including hairpin and hammerhead ribozymes, have been identified and extensively characterized. Examples of cleaving ribozymes and uses thereof are described in international application WO 2005/049817 A2, the contents of which are incorporated by reference herein.

### Activator Receptors

The disclosure provides an activator receptor, wherein the activator receptor is split into two portions, each portion encoded by a separate vector as described herein.

In some embodiments, the activator receptor is a CAR.

In some embodiments, for example those embodiments where the activator receptor comprises a split antigen binding domain, the activator receptor comprises an antigen binding domain, a hinge domain, first transmembrane domain and intracellular domain. In some embodiments, the first portion of the activator receptor comprises a first portion of the antigen binding domain, and the second portion of the activator receptor comprises a second portion of the antigen binding domain, the hinge domain, the transmembrane domain, and the intracellular domain. Suitable split antigen binding domains are described supra.

In some embodiments, for example those embodiments where first and second portions of the activator receptor are associated through the hinge, or hinge and transmembrane domains, the first portion of the activator receptor comprises the antigen binding domain, a first hinge domain, and a first transmembrane domain, and the second portion of the activator receptor comprises a second hinge domain, a second transmembrane domain, and an intracellular domain. In some embodiments, the first and second hinge domains are the same, e.g. CD8α hinges. In some embodiments, the first and second hinge domains are not the same, e.g. Fos and Jun derived hinges.

Antigen binding domains of the activator receptors of the disclosure may include, but are not limited to, fragment antigen-binding (Fab) fragments, single chain Fab (scFab) fragments, F(ab′)2 fragments, Fab′ fragments, Fv fragments, recombinant IgG (rIgG) fragments, single chain antibody fragments, single chain variable fragments (scFv), single domain antibodies (e.g., sdAb, sdFv, nanobody) fragments, diabodies, and multi-specific antibodies formed from antibody fragments. In particular embodiments, the antigen binding domains are single-chain antibody fragments comprising a variable heavy chain region and/or a variable light chain region, such as scFvs.

In some embodiments, the antigen-binding domain may comprise a scFv having a VH-linker-VL orientation. In some embodiments, the antigen-binding domain may comprise a scFv having a VL-linker-VH orientation.

In some embodiments, the antigen binding domain of the receptor is a scFv.

In some embodiments, the antigen-binding domain further comprises a leader sequence or signal peptide. In embodiments where the antigen-binding domain comprises a scFv, the signal peptide may be positioned at the amino terminus of the scFv. In some embodiments, when the heavy chain variable region is N-terminal, the signal peptide may be positioned at the amino terminus of the heavy chain variable region. In some embodiments, when the light chain variable region is N-terminal, the signal peptide may be positioned at the amino terminus of the light chain variable region. The signal peptide may comprise any suitable signal peptide. In some embodiments, the signal peptide comprises the sequence of MDMRVPAQLLGLLLLWLRGARC.

In some embodiments, the activator is specific to cancer antigen, i.e. the activator receptor activates one or more functions of the cell in which it expressed upon binding to its cognate antigen. As used herein, a “cancer antigen” refers to an antigen expressed by a target cell that represents a potential therapeutic target.

In some embodiments, the activator receptor is specific to a target antigen selected from the group of TNF receptor superfamily member 17 (BCMA), BCR-Abl, bone marrow stromal cell antigen 2 (BST2), carbonic anhydrase 9 (CAIX), CD19 molecule (CD19), CD20, CD22, CD123, CD171, CD30, CD33, CD38, CD44v6, CD44v7/8, CEA cell adhesion molecule (CEACAM, or CEA), CEACAM1, CEACAM3, CEACAM5, C-type lectin domain family 12 member A (CLL-1), epidermal growth factor receptor (EGFR), EGFRvIII, epithelial cell adhesion molecule (EGP-2), erb-b2 receptor tyrosine kinase 2 (ERBB2, or Her2/neu), EPCAM, fetal acetylcholine receptor, fins related receptor tyrosine kinase 3 (FLT3), Folate receptor alpha (FBP), Disialoganglioside GD2 (GD2), disialoganglioside GD3 (GD3), Her3 (ErbB3), Her4 (ErbB4), k-light chain, kinase insert domain receptor (KDR), MAD-CT-1, MAD-CT-2, MAGE-A1, MAGE-A3, melan-A (MARTI), ML-IAP, MYCN, Oncofetal antigen (h5T4), NKG2D ligands, pyruvate dehydrogenase kinase 1 (PDKI), PDL1, PSCA, PSMA, PRSS21, ROR1, SLAMF7, TAG-72, Tn Ag, TSLPR, B7H3 (CD276), KIT (CD17), IL-13Ra2, Mesothelin (MSLN), IL-11Ra, VEGFR2, LeY, CD24, PDGFR-beta, SSEA-4; CD20, MUC1, epidermal growth factor receptor (EGFR), NCAM, Prostase, PAP, ELF2M, Ephrin B2, FAP, IGF-1 receptor, CAFX, LMP2, gp100, tyrosinase, EphA2, Fucosyl GM1, sLe, ganglioside GM3, TGS5, HMWMAA, OAcGD2, OR51E2, Folate receptor beta, TEM1/CD248, TEM7R, CLDN6, TSHR, GloboH, GPR20, GPRC5D, CXORF61, CD97, CD179a, ADRB3, ALK, Polysialic acid, PANX3, PLAC1, NY-BR-1, NY-ESO-1, UPK2, TIM-1, HAVCR1, LY6K, TARP, WT1, LAGE-1a, ETV6-AML, SPA17, XAGEl, Tie 2, Fos-related antigen 1, p53, p53 mutant, prostein, survivin, telomerase, PCTA-1, Rat sarcoma Ras mutant, hTERT, sarcoma translocation breakpoints, ERG, NA17, PAX3, Androgen receptor, Cyclin B1, RhoC, TRP-2, CYP1B1, BORIS, SART3, PAX5, OY-TESI, LCK, AKAP-4, SSX2, RAGE-1, RU1, RU2, legumain, HPV E6, HPV E7, intestinal carboxyl esterase, mut hsp70-2, CD79a, CD79b, CD72, LAIR1, CD89, LILRA2, CD300LF, CLEC12A, EMR2, FCRL5, GPC3, IGLL1, and LY75.

In some embodiments, the CAR comprises a linker, spacer, or hinge sequence between the extracellular antigen binding domain and the transmembrane domain. One of ordinary skill in the art will appreciate that a hinge sequence is a short sequence of amino acids that, in at least some instances, facilitates flexibility (see, e.g., Woof et al., Nat. Rev. Immunol., 4(2): 89-99 (2004)).

In those embodiments where the receptor is split via the antigen binding domain, the hinge sequence can be any suitable sequence derived or obtained from any suitable molecule. The hinge may be derived from or include at least a portion of an immunoglobulin Fc region, for example, an IgG1 Fc region, an IgG2 Fc region, an IgG3 Fc region, an IgG4 Fc region, an IgE Fc region, an IgM Fc region, or an IgA Fc region. In certain embodiments, the spacer domain includes at least a portion of an IgG1, an IgG2, an IgG3, an IgG4, an IgE, an IgM, or an IgA immunoglobulin Fc region that falls within its CH2 and CH3 domains. In some embodiments, the spacer domain may also include at least a portion of a corresponding immunoglobulin hinge region. In some embodiments, the hinge is derived from or includes at least a portion of a modified immunoglobulin Fc region, for example, a modified IgG1 Fc region, a modified IgG2 Fc region, a modified IgG3 Fc region, a modified IgG4 Fc region, a modified IgE Fc region, a modified IgM Fc region, or a modified IgA Fc region. The modified immunoglobulin Fc region may have one or more mutations (e.g., point mutations, insertions, deletions, duplications) resulting in one or more amino acid substitutions, modifications, or deletions that cause impaired binding of the spacer domain to an Fc receptor (FcR). In some aspects, the modified immunoglobulin Fc region may be designed with one or more mutations which result in one or more amino acid substitutions, modifications, or deletions that cause impaired binding of the spacer domain to one or more FcR including, but not limited to, FcγRI, FcγR2A, FcγR2B1, FcγR2B2, FcγR3A, FcγR3B, FcεRI, FcεR2, FcαRI, Fcα/μR, or FcRn.

In some embodiments, a portion of the immunoglobulin constant region serves as a hinge region between the antigen binding domain, e.g., scFv, and transmembrane domain. The spacer can be of a length that provides for increased responsiveness of the cell following antigen binding, as compared to in the absence of the spacer. Exemplary hinges include those having at least about 10 to 229 amino acids, about 10 to 200 amino acids, about 10 to 175 amino acids, about 10 to 150 amino acids, about 10 to 125 amino acids, about 10 to 100 amino acids, about 10 to 75 amino acids, about 10 to 50 amino acids, about 10 to 40 amino acids, about 10 to 30 amino acids, about 10 to 20 amino acids, or about 10 to 15 amino acids, and including any integer between the endpoints of any of the listed ranges. In some embodiments, a hinge has about 12 amino acids or less, about 119 amino acids or less, or about 229 amino acids or less. In some embodiments, the hinge is at or about 12 amino acids in length. Exemplary hinges include a CD28 hinge, IgG4 hinge alone, IgG4 hinge linked to CH2 and CH3 domains, or IgG4 hinge linked to the CH3 domain. Exemplary spacers include, but are not limited to, those described in Hudecek et al. (2013) Clin. Cancer Res., 19:3153, international patent application publication number WO2014031687, U.S. Pat. No. 8,822,647 or published app. No. US2014/0271635.

In some embodiments, the CAR comprises a hinge domain isolated or derived from CD4, CD8α, IgG1, IgG2, or IgG4. In some embodiments, the hinge domain is isolated or derived from the human CD8α molecule or a CD28 molecule. In some embodiments, the hinge sequence is isolated or derived from CD8α. In some embodiments, the hinge is isolated or derived from HLA-A.

In some embodiments, the hinge is isolated or derived from CD8α. In some embodiments, the CD8α hinge comprises an amino acid sequence having at least 80% identity, at least 90% identity, at least 95% identity, at least 99% identity or is identical to a sequence of TTTPAPRPPTPAPTIASQPLSLRPEACRPAAGGAVHTRGLDFACD. In some embodiments, the CD8α hinge comprises TTTPAPRPPTPAPTIASQPLSLRPEACRPAAGGAVHTRGLDFACD (SEQ ID NO: 102). In some embodiments, the CD8α hinge consists essentially of TTTPAPRPPTPAPTIASQPLSLRPEACRPAAGGAVHTRGLDFACD.

TTTPAPRPPTPAPTIASQPLSLRPEACRPAAGGAVHTRGLDFACD.

In some embodiments, the hinge is isolated or derived from CD28. In some embodiments, the CD28 hinge comprises an amino acid sequence having at least 90% identity, at least 95% identity, at least 99% identity or is identical to a sequence of CTIEVMYPPPYLDNEKSNGTIIHVKGKHLCPSPLFPGPSKP. In some embodiments, the CD28 hinge comprises or consists essentially of

CTIEVMYPPPYLDNEKSNGTIIHVKGKHLCPSPLFPGPSKP.

Optionally, a polypeptide linker may form the linkage between the transmembrane domain and the intracellular signaling domain(s) of the CAR. A glycine-seine doublet may provide a suitable linker. Alternatively, or in addition, poly-glycine and poly-serine sequences may provide suitable linkers. In some embodiments, the polypeptide linker is between 2 and 10 amino acids in length.

With respect to the transmembrane domain, the CAR can be designed to comprise a transmembrane domain that is fused to the antigen-binding domain of the CAR. In some embodiments, the transmembrane domain that naturally is associated with one of the domains in the CAR is used. In some instances, the transmembrane domain can be selected or modified by amino acid substitution to avoid binding of such domains to the transmembrane domains of the same or different surface membrane proteins to minimize interactions with other members of the receptor complex.

The transmembrane domain may be derived either from a natural or from a synthetic source. Where the source is natural, the domain may be derived from any membrane-bound or transmembrane protein. Typically, the transmembrane domain denotes a single transmembrane alpha helix of a transmembrane protein, also known as an integral protein. Transmembrane regions of particular use in this invention may be derived from (i.e. comprise at least the transmembrane region(s) of) CD28, CD3 epsilon, CD4, CD5, CD8, CD9, CD16, CD22, CD33, CD37, CD45, CD64, CD80, CD86, CD134, CD137, CD154, TCR alpha, TCR beta, or CD3 zeta and/or transmembrane regions containing functional variants thereof such as those retaining a substantial portion of the structural, e.g., transmembrane, properties thereof.

In some embodiments, the transmembrane domain comprises a comprises a transmembrane domain isolated or derived from CD8α molecule (CD8α), CD4 molecule (CD4), CD28 molecule (CD28), TNF receptor superfamily member 9 (CD137, or 4-1BB), CD80 molecule (CD80), CD86 molecule (CD86), cytotoxic T-lymphocyte associated protein 4 (CD152), programmed cell death 1 (PD-1), CD247 molecule (CD3ζ), or Fc fragment of IgE receptor Ig (FcRγ).

Alternatively, the transmembrane domain may be synthetic, in which case it will comprise predominantly hydrophobic residues such as leucine and valine. In some embodiments, a triplet of phenylalanine, tryptophan and valine can be used at one or both ends of a synthetic transmembrane domain. A transmembrane domain of the invention can be thermodynamically stable in a membrane. It may be a single alpha helix, a transmembrane beta barrel, a beta-helix of gramicidin A, or any other structure. In some embodiments, transmembrane helices are about 20 amino acids in length.

In some embodiments, the transmembrane domain in the CAR of the invention is the CD28 transmembrane domain.

In some embodiments, the transmembrane is isolated or derived from HLA-A. In some embodiments, the HLA-A transmembrane domain comprises an amino acid sequence having at least, at least 90% identity, at least 95% identity, at least 99% identity or is identical to a sequence of VGIIAGLVLFGAVITGAVVAAVMWRSKRSR. In some embodiments, the HLA-A transmembrane domain comprises or consists essentially of

VGIIAGLVLFGAVITGAVVAAVMWRSKRSR.

In those embodiments wherein the CAR is an activator receptor, the intracellular signaling domain or otherwise the cytoplasmic domain of the CAR of the invention triggers or elicits activation of at least one of the normal effector functions of the immune cell in which the CAR has been placed. For example, the intracellular domain can be isolated or derived from an immune effector cell protein. The term “effector function” refers to a specialized function of a cell. Effector function of a T cell, for example, may be cytolytic activity or helper activity including the secretion of cytokines. Thus, the term “intracellular signaling domain” refers to the portion of a protein which transduces the effector function signal and directs the cell to perform a specialized function. While the entire intracellular signaling domain may be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

Preferred examples of intracellular signaling domains for use in the activator CARs of the disclosure include the cytoplasmic sequences of the T cell receptor (TCR) and co-receptors that act in concert to initiate signal transduction following antigen receptor engagement, as well as any derivative or variant of these sequences and any synthetic sequence that has the same functional capability.

Signals generated through one intracellular signaling domain alone may be insufficient for full activation of an immune cell, and a secondary or co-stimulatory signal may also be required. For example, T cell activation can be said to be mediated by two distinct classes of cytoplasmic signaling sequence: those that initiate antigen-dependent primary activation through the TCR (primary cytoplasmic signaling sequences) and those that act in an antigen-independent manner to provide a secondary or co-stimulatory signal (secondary cytoplasmic signaling sequences). Primary cytoplasmic signaling sequences regulate primary activation of the TCR complex either in a stimulatory way, or in an inhibitory way. Primary cytoplasmic signaling sequences that act in a stimulatory manner may contain signaling motifs which are known as immunoreceptor tyrosine-based activation motifs or ITAMs.

Examples of ITAM-containing primary cytoplasmic signaling sequences that are useful as intracellular signaling domains according to the present disclosure include those derived from an intracellular signaling domain of a lymphocyte receptor chain, a TCR/CD3 complex protein, an Fc receptor subunit, an IL-2 receptor subunit, CD3ζ, FcRγ, FcRβ, CD3γ, CD3δ, CD3ε, CD5, CD22, CD79a, CD79b, CD66d, CD278(ICOS), FcεRI, DAP10, and DAP12. In some embodiments, the intracellular signaling domain in the CAR of the invention comprises a cytoplasmic signaling sequence derived from CD3. In some embodiments, the intracellular domain comprises an intracellular domain isolated or derived from CD3ζ.

In some embodiments of the activator CARs of the disclosure, the CAR comprises a costimulatory domain. The costimulatory domain refers to a portion of the CAR comprising the intracellular domain of a costimulatory molecule. A costimulatory molecule is a cell surface molecule other than an antigen receptor or their ligands that is required for an efficient response of lymphocytes to an antigen.

Various co-stimulatory domains have been reported to confer differing properties. For example, the 4-1BB co-stimulatory domain showed enhanced persistence in in vivo xenograph models (Milone et al. Mol Ther 2009; 17:1453-1464; Song et al. Cancer Res 2011; 71:4617-4627). Additionally, these different co-stimulatory domains produce different cytokine profiles which, in turn, may produce effects on target cell-mediated cytotoxicity and the tumor microenvironment. DAP10 signaling in NK cells has been associated with an increase in Th1 and inhibition of Th2 type cytokine production in CD8+ T cells (Barber et al. Blood 2011; 117:6571-6581).

Non-limiting examples of co-stimulatory molecules include an MHC class I molecule, TNF receptor proteins, immunoglobulin-like proteins, cytokine receptors, integrins, signaling lymphocytic activation molecules (SLAM proteins), NK cell receptors, a Toll ligand receptor, B7-H3, BAFFR, BTLA, BLAME (SLAMF8), CD2, CD4, CD5, CD7, CD8alpha, CD8beta, CD11a, LFA-1 (CD11a/CD18), CD11b, CD11c, CD11d, CD18, CD19, CD19a, CD27, CD28, CD29, CD30, CD40, CD49a, CD49D, CD49f, CD69, CD84, CD96 (Tactile), CD100 (SEMA4D), CD103, CRTAM, OX40 (CD134), 4-1BB (CD137), SLAM (SLAMF1, CD150, IPO-3), CD160 (BY55), SELPLG (CD162), DNAM1 (CD226), Ly9 (CD229), SLAMF4 (CD244, 2B4), ICOS (CD278), CEA cell adhesion molecule (CEACAM, or CEA), CEACAM1, CEACAM3, CEACAM5, CDS, CRTAM, DAP10, GADS, GITR, HVEM (LIGHTR), IA4, ICAM-1, IL2R beta, IL2R gamma, IL7R alpha, ITGA4, ITGA6, ITGAD, ITGAE, ITGAL, ITGAM, ITGAX, ITGB1, ITGB2, ITGB7, KIRDS2, LAT, LFA-1, LIGHT, LTBR, NKG2C, NKG2D, NKp30, NKp44, NKp46, NKp80 (KLRF1), PAG/Cbp, PD-1, PSGL1, SLAMF6 (NTB-A, Ly108), SLAMF7, SLP-76, TNFR2, TRANCE/RANKL, VLA1, VLA-6, a ligand that specifically binds with CD83, and the like.

Thus, while the activator CARs are exemplified primarily with regions of CD28, and/or 4-1BB as the co-stimulatory signaling elements, other costimulatory elements are within the scope of the disclosure. In some embodiments, the co-stimulatory domain comprises a co-stimulatory domain isolated or derived from CD27 molecule (CD27), CD28, CD137, TNF receptor superfamily member 4 (OX40), TNF receptor superfamily member 8 (CD30), CD40 molecule (CD40), CD40 ligand (CD40L), CD3ζ, integrin subunit beta 2 (LFA-1), inducible T cell costimulator (ICOS), CD2 molecule (CD2), CD7 molecule (CD7), TNF superfamily member 14 (LIGHT), killer cell lectin like receptor C2 (NKG2C), CD276 molecule (B7-H3), or hematopoietic cell signal transducer (DAP10).

The cytoplasmic signaling sequences within the intracellular signaling domain of the CAR of the invention may be linked to each other in a random or specified order. Optionally, a short oligo- or polypeptide linker, may form the linkage. In some embodiments, the linker comprises glycine-serine doublet. In some embodiments, the linker is between 2 and 10 amino acids in length.

In some embodiments, the intracellular domain comprises the intracellular signaling domain of CD3ζ and a costimulatory domain derived from CD28. In some embodiments, the intracellular domain comprises the intracellular signaling domain of CD3ζ and a costimulatory domain derived from 4-1BB. In some embodiments, the intracellular domain comprises the intracellular signaling domain of CD3-ζ and costimulatory domains derived from both CD28 and 4-1BB.

In some embodiments, the hinge, transmembrane, and intracellular domains of the CAR comprise a sequence at least 90%, at least 95%, at least 99%, or 100% identical to

TTTPAPRPPTPAPTIASQPLSLRPEACRPAAGGAVHTRGLDFACDFWVLV
VVGGVLACYSLLVTVAFIIFWVRSKRSRLLHSDYMNMTPRRPGPTRKHYQ
PYAPPRDFAAYRSKRGRKKLLYIFKQPFMRPVQTTQEEDGCSCRFPEEEE
GGCELRVKFSRSADAPAYKQGQNQLYNELNLGRREEYDVLDKRRGRDPEM
GGKPRRKNPQEGLYNELQKDKMAEAYSEIGMKGERRRGKGHDGLYQGLST
ATKDTYDALHMQALPPR.

Included in the scope of the disclosure are functional variants of the CARs described herein. The term “functional variant” as used herein refers to a CAR, polypeptide, or protein having substantial or significant sequence identity or similarity to a parent CAR, which functional variant retains the biological activity of the CAR of which it is a variant. Functional variants encompass those variants of the CAR described herein (the parent CAR) that retain the ability to recognize target cells to a similar extent, the same extent, or to a higher extent, as the parent CAR. In reference to the parent CAR, the functional variant can, for instance, be at least about 30%, 50%, 75%, 80%, 90%, 95%, 98%, 99% or 100% identical in amino acid sequence to the parent CAR. As is well known in the art, functional variants of antibodies, and by extension CARs comprising antibody fragments or antigen binding domains derived from antibodies, can be generated by varying the amino acids in the so-called framework regions of the antibody. Those of skill in the art can, through sequence alignment or structural modeling, identify residues that may be varied without loss of function and may select appropriate amino acid substitutions. Substitutions may include substitutions in solvent-exposed resides, often identified as non-conserved, polar, or hydrophilic. Interior residues may be varied by conservative substitutions—e.g., of one hydrophobic residue for another, or of residues of similar size or similar backbone rotational freedom. Structural models of representative VH and VL families have been published. Therefore it is possible, without undue effort, to identify reasonable substitutions to make based on such published structures. These techniques may be used to humanize antibodies. Furthermore, using methods disclosed herein or known in the art, one may with routine experimentation confirm that variants retain their desired function—e.g., specific binding to a target. Functional variants of antibodies are not limited to substitution in the framework regions. For example, CDR sequences may be swapped between antibodies having a common target specificity and tested with routine experimentation. CDR sequences are not fixed either. Point mutations or insertion may be designed using techniques well known in the art, and testing of such variants is routine. Moreover, VH-VL pairings can be varied. The preferred pairing partners for each VH and VL type are well known. Thus, is it not necessary to test every possible VH and VL pair. Rather, those skilled in the art are capable of rationally selecting candidate pairings. For all these reasons, the scope of the present disclosure is not intended to limit the invention to only the exact amino acid sequences disclosed herein. In particular, polypeptides of the disclosure may have amino acid substitutions and insertion in the framework regions of the antigen binding domain so long as the function of the antigen binding domain is retained, which could be confirm with routine experimentation using isolated antigen binding domains or functional CARs expressed in cells.

A functional variant can, for example, comprise the amino acid sequence of the parent CAR with at least one conservative amino acid substitution. Alternatively or additionally, the functional variants can comprise the amino acid sequence of the parent CAR with at least one non-conservative amino acid substitution. In this case, it is preferable for the non-conservative amino acid substitution to not interfere with or inhibit the biological activity of the functional variant. The non-conservative amino acid substitution may enhance the biological activity of the functional variant, such that the biological activity of the functional variant is increased as compared to the parent CAR.

Amino acid substitutions of the CARs of the disclosure are preferably conservative amino acid substitutions. Conservative amino acid substitutions are known in the art, and include amino acid substitutions in which one amino acid having certain physical and/or chemical properties is exchanged for another amino acid that has the same or similar chemical or physical properties. For instance, the conservative amino acid substitution can be an acidic/negatively charged polar amino acid substituted for another acidic/negatively charged polar amino acid (e.g., Asp or Glu), an amino acid with a nonpolar side chain substituted for another amino acid with a nonpolar side chain (e.g., Ala, Gly, Val, Ile, Leu, Met, Phe, Pro, Trp, Cys, Val, etc.), a basic/positively charged polar amino acid substituted for another basic/positively charged polar amino acid (e.g. Lys, His, Arg, etc.), an uncharged amino acid with a polar side chain substituted for another uncharged amino acid with a polar side chain (e.g., Asn, Gin, Ser, Thr, Tyr, etc.), an amino acid with a beta-branched side-chain substituted for another amino acid with a beta-branched side-chain (e.g., Ile, Thr, and Val), an amino acid with an aromatic side-chain substituted for another amino acid with an aromatic side chain (e.g., His, Phe, Trp, and Tyr), etc.

Also, amino acids may be added or removed from the sequence based on vector design.

The activator receptors can consist essentially of the specified amino acid sequence or sequences described herein, such that other components, e.g., other amino acids, do not materially change the biological activity of the functional variant.

Receptors (including functional portions and functional variants thereof) can be obtained by methods known in the art. The receptors may be made by any suitable method of making polypeptides or proteins. Suitable methods of de novo synthesizing polypeptides and proteins are described in references, such as Chan et al., Fmoc Solid Phase Peptide Synthesis, Oxford University Press, Oxford, United Kingdom, 2000; Peptide and Protein Drug Analysis, ed. Reid, R., Marcel Dekker, Inc., 2000; Epitope Mapping, ed. Westwood et al., Oxford University Press, Oxford, United Kingdom, 2001; and U.S. Pat. No. 5,449,752. Also, polypeptides and proteins can be recombinantly produced using the nucleic acids described herein using standard recombinant methods. See, for instance, Sambrook et al., Molecular Cloning: A Laboratory Manual, 3rd ed., Cold Spring Harbor Press, Cold Spring Harbor, N.Y. 2001; and Ausubel et al., Current Protocols in Molecular Biology, Greene Publishing Associates and John Wiley & Sons, N Y, 1994. Further, some of the CARs of the invention (including functional portions and functional variants thereof) can be isolated and/or purified from a source, such as a plant, a bacterium, an insect, a mammal, e.g., a rat, a human, etc. Methods of isolation and purification are well-known in the art. Alternatively, the CARs described herein (including functional portions and functional variants thereof) can be commercially synthesized by companies. In this respect, the receptors can be synthetic, recombinant, isolated, and/or purified.

The activator receptors of the disclosure can be TCRs. Antigen binding domains, for example antigen binding domains specific to a cancer antigen, can be fused to any one or more of the TCRα, TCRβ, CD3ζ, CD3δ, CD3ε or CD3γ subunits of the TCR. For example, all or part of the endogenous extracellular antigen binding domain of TCRα and/or TCRβ can be replaced with an antigen binding domain fused to the TCRα and TCRβ subunits, which are encoded separately by the dual vectors described herein.

In some embodiments, the TCR further comprises one or more additional intracellular domains that enhance and or alter the activity of the TCR. Additional intracellular domains can be fused to any one or more of TCRα, TCRβ, CD3ζ, CD3δ, CD3ε or CD3γ subunits of the TCR, in place of, or in addition to, the endogenous intracellular domain of the TCR subunit.

In some embodiments, the TCR comprises an intracellular domain that provides an activator signal to an immune cell expressing the TCR, thereby enhancing the activity of the TCR. TCRs comprising additional activator domains are described in WO 2021/030153, the contents of which are incorporated by reference in their entirety herein. Exemplary activator domains for use with the TCRs of the disclosure include, but are not limited to, domains isolated or derived from CD28 molecule (CD28), TNF receptor superfamily member 9 (4-1BB), TNF receptor superfamily member 18 (GITR), LCK proto-oncogene, Src family tyrosine kinase (Lck), CD4 molecule (CD4), CD8α molecule (CD8), FYN proto-oncogene, Src family tyrosine kinase (Fyn), zeta chain of T cell receptor associated protein kinase 70 (ZAP70), linker for activation of T cells (LAT), and lymphocyte cytosolic protein 2 (SLP76).

Exemplary intracellular domain sequences are provided in Table 1 below.

TABLE 1
Exemplary Activator CAR Intracellular Domain Sequences

| IC Domain | Protein Sequence | DNA Sequence |
|---|---|---|
| CD28-4- | RSKRSRLLHSDYMN | AGGAGTAAGAGGAGCAGGCTCCTGCACAGTGACTACATGAAC |
| 1BB-CD3z | MTPRRPGPTRKHYQ | ATGACTCCCCGCCGCCCCGGGCCCACCCGCAAGCATTACCAG |
| | PYAPPRDFAAYRSR | CCCTATGCCCCACCACGCGACTTCGCAGCCTATCGCTCCCGT |
| | FSVVKRGRKKLLYI | TTCTCTGTTGTTAAACGGGGCAGAAAGAAGCTCCTGTATATA |
| | FKQPFMRPVQTTQE | TTCAAACAACCATTTATGAGACCAGTACAAACTACTCAAGAG |
| | EDGCSCRFPEEEEG | GAAGATGGCTGTAGCTGCCGATTTCCAGAAGAAGAAGAAGGA |
| | GCELRVKFSRSADA | GGATGTGAACTGAGAGTGAAGTTCAGCAGGAGCGCAGACGCC |
| | PAYQQGQNQLYNEL | CCCGCGTACCAGCAGGGCCAGAACCAGCTCTATAACGAGCTC |
| | NLGRREEYDVLDKR | AATCTAGGACGAAGAGAGGAGTACGATGTTTTGGACAAGAGA |
| | RGRDPEMGGKPRRK | CGTGGCCGGGACCCTGAGATGGGGGGAAAGCCGAGAAGGAAG |
| | NPQEGLYNELQKDK | AACCCTCAGGAAGGCCTGTACAATGAACTGCAGAAAGATAAG |
| | MAEAYSEIGMKGER | ATGGCGGAGGCCTACAGTGAGATTGGGATGAAAGGCGAGCGC |
| | RRGKGHDGLYQGLS | CGGAGGGGCAAGGGGCACGATGGCCTTTACCAGGGTCTCAGT |
| | TATKDTYDALHMQA | ACAGCCACCAAGGACACCTACGACGCCCTTCACATGCAGGCC |
| | LPPR | CTGCCCCCTCGCTAG |
| CD28 | FWVLVVVGGVLACY | TTCTGGGTGCTGGTCGTTGTGGGCGGCGTGCTGGCCTGCTAC |
| hinge + | SLLVTVAFIIFWVR | AGCCTGCTGGTGACAGTGGCCTTCATCATCTTTTGGGTGAGG |
| TM, 4-1BB + | SKRSRLLHSDYMNM | AGCAAGCGGAGCAGACTGCTGCACAGCGACTACATGAACATG |
| CD3Z | TPRRPGPTRKHYQP | ACCCCCCGGAGGCCTGGCCCCACCCGGAAGCACTACCAGCCC |
| ICD (CD28 | YAPPRDFAAYRSKR | TACGCCCCTCCCAGGGATTTCGCCGCCTACCGGAGCAAACGG |
| hinge + | GRKKLLYIFKQPFM | GGCAGAAAGAAACTCCTGTATATATTCAAACAACCATTTATG |
| TM is | RPVQTTQEEDGCSC | AGGCCAGTACAAACTACTCAAGAGGAAGATGGCTGTAGCTGC |
| bold, | RFPEEEEGGCELRV | CGATTTCCAGAAGAAGAAGAAGGAGGATGTGAACTGAGAGTG |
| CD3z is | KFSRSADAPAYKQG | AAGTTCAGCAGGAGCGCAGACGCCCCCGCGTACAAGCAGGGC |
| underlined) | QNQLYNELNLGRRE | CAGAACCAGCTCTATAACGAGCTCAATCTAGGACGAAGAGAG |
| | EYDVLDKRRGRDPE | GAGTACGATGTTTTGGACAAGCGTAGAGGCCGGGACCCTGAG |
| | MGGKPRRKNPQEGL | ATGGGGGGAAAGCCGAGAAGGAAGAACCCTCAGGAAGGCCTG |
| | YNELQKDKMAEAYS | TACAATGAACTGCAGAAAGATAAGATGGCGGAGGCCTACAGT |
| | EIGMKGERRRGKGH | GAGATTGGGATGAAAGGCGAGCGCCGGAGGGGCAAGGGGCAC |
| | DGLYQGLSTATKDT | GATGGCCTTTACCAGGGACTCAGTACAGCCACCAAGGACACC |
| | YDALHMQALPPR | TACGACGCCCTTCACATGCAGGCCCTGCCCCCTCGC |
| CD28-CD3z | RSKRSRLLHSDYMN | AGGAGCAAGCGGAGCAGACTGCTGCACAGCGACTACATGAAC |
| | MTPRRPGPTRKHYQ | ATGACCCCCCGGAGGCCTGGCCCCACCCGGAAGCACTACCAG |
| | PYAPPRDFAAYRSR | CCCTACGCCCCTCCCAGGGATTTCGCCGCCTACCGGAGCAGA |
| | VKFSRSADAPAYKQ | GTGAAGTTCAGCAGGAGCGCAGACGCCCCCGCGTACAAGCAG |
| | GQNQLYNELNLGRR | GGCCAGAACCAGCTCTATAACGAGCTCAATCTAGGACGAAGA |
| | EEYDVLDKRRGRDP | GAGGAGTACGATGTTTTGGACAAGCGTAGAGGCCGGGACCCT |
| | EMGGKPRRKNPQEG | GAGATGGGGGGAAAGCCGAGAAGGAAGAACCCTCAGGAAGGC |
| | LYNELQKDKMAEAY | CTGTACAATGAACTGCAGAAAGATAAGATGGCGGAGGCCTAC |
| | SEIGMKGERRRGKG | AGTGAGATTGGGATGAAAGGCGAGCGCCGGAGGGGCAAGGGG |
| | HDGLYQGLSTATKD | CACGATGGCCTTTACCAGGGACTCAGTACAGCCACCAAGGAC |
| | TYDALHMQALPPR | ACCTACGACGCCCTTCACATGCAGGCCCTGCCCCCTCGCTAG |
| 4-1BB- | KRGRKKLLYIFKQP | AAACGGGGCAGAAAGAAGCTCCTGTATATATTCAAACAACCA |
| CD3z | FMRPVQTTQEEDGC | TTTATGAGACCAGTACAAACTACTCAAGAGGAAGATGGCTGT |
| | SCRFPEEEEGGCEL | AGCTGCCGATTTCCAGAAGAAGAAGAAGGAGGATGTGAACTG |
| | GGGRVKFSRSADAP | GGCGGTGGCAGAGTGAAGTTCAGCAGGAGCGCAGACGCCCCC |
| | AYQQGQNQLYNELN | GCGTACCAGCAGGGCCAGAACCAGCTCTATAACGAGCTCAAT |
| | LGRREEYDVLDKRR | CTAGGACGAAGAGAGGAGTACGATGTTTTGGACAAGAGACGT |
| | GRDPEMGGKPRRKN | GGCCGGGACCCTGAGATGGGGGGAAAGCCGAGAAGGAAGAAC |
| | PQEGLYNELQKDKM | CCTCAGGAAGGCCTGTACAATGAACTGCAGAAAGATAAGATG |
| | AEAYSEIGMKGERR | GCGGAGGCCTACAGTGAGATTGGGATGAAAGGCGAGCGCCGG |
| | RGKGHDGLYQGLST | AGGGGCAAGGGGCACGATGGCCTTTACCAGGGTCTCAGTACA |
| | ATKDTYDALHMQAL | GCCACCAAGGACACCTACGACGCCCTTCACATGCAGGCCCTG |
| | PPR | CCCCCTCGCTAG |

### Inhibitory Receptor

The disclosure provides inhibitory receptors, encoded by one of the dual vectors of the vector system described herein. In some embodiments, the inhibitory receptor comprises an extracellular antigen binding domain, a transmembrane domain, and an inhibitory intracellular domain. In some embodiments, the inhibitory receptor further comprises a hinge domain. In some embodiments, the antigen binding domain is specific to an antigen whose expression is lost in a cancer cell through loss of heterozygosity.

The disclosure provides an inhibitory receptor, such as an inhibitory CAR or TCR. In some embodiments, the inhibitory receptor comprises an extracellular antigen binding domain specific to a non-target antigen, sometimes referred to herein as a second antigen or inhibitor antigen, that is not expressed by cancer cells due to a loss of heterozygosity in the cancer cells. In some embodiments, the non-target antigen comprises an HLA class I allele or a minor histocompatibility antigen (MiHA). In some embodiments, the HLA Class I allele comprises HLA-A, HLA-B, HLA-C or HLA-E. In some embodiments, the HLA class I allele comprises HLA-A*02.

Antigen binding domains of the inhibitory receptors of the disclosure may include, but are not limited to, fragment antigen-binding (Fab) fragments, single chain Fab (scFab) fragments, F(ab′)2 fragments, Fab′ fragments, Fv fragments, recombinant IgG (rIgG) fragments, single chain antibody fragments, single chain variable fragments (scFv), single domain antibodies (e.g., sdAb, sdFv, nanobody) fragments, diabodies, and multi-specific antibodies formed from antibody fragments. In particular embodiments, the antigen binding domains are single-chain antibody fragments comprising a variable heavy chain region and/or a variable light chain region, such as scFvs.

In some embodiments, the inhibitory receptor comprises an extracellular antigen binding domain specific to a non-target antigen that is not expressed by the cancer cell due to a loss of heterozygosity in the cancer cell.

In some embodiments, the inhibitor antigen is a peptide antigen. In some embodiments, the inhibitor antigen is a peptide antigen complexed with a major histocompatibility (MHC) class I complex (peptide MHC, or pMHC). Inhibitor antigens comprising peptide antigens complexed with pMHC comprising any of HLA-A, HLA-B, or HLA-C are envisaged as within the scope of the disclosure.

In some embodiment, the inhibitor antigen is encoded by a gene that is absent or polymorphic in many tumors.

Methods of distinguishing the differential expression of inhibitor antigens between target and non-target cells will be readily apparent to the person or ordinary skill in the art. For example, the presence or absence of inhibitor antigens in non-target and target cells can be assayed by immunohistochemistry with an antibody that binds to the inhibitor antigen, followed by microscopy or FACS, RNA expression profiling of target cells and non-target cells, or DNA sequencing of non-target and target cells to determine if the genomic locus of the inhibitor antigen comprises mutations in either the target or non-target cells.

In some embodiments, the second, inhibitor antigen comprises an allele of a gene that is lost in target cells due to loss of heterozygosity. In some embodiments, the target cells comprise cancer cells. Cancer cells undergo frequent genome rearrangements, including duplication and deletions. These deletions can lead to the deletion of one copy of one or more genes in the cancer cells.

As used herein, “loss of heterozygosity (LOH)” refers to a genetic change that occurs at high frequency in cancers, whereby one of the two alleles is deleted, leaving a single mono-allelic (hemizygous) locus.

In some embodiments, the second, inhibitor antigen comprises an HLA class I allele. The major histocompatibility complex (MHC) class I is a protein complex that displays antigens to cells of the immune system, triggering immune response. The Human Leukocyte Antigens (HLAs) corresponding to MHC class I are HLA-A, HLA-B, HLA-C, HLA-E, HLA-F and HLA-G.

In some embodiments, the second, inhibitor antigen comprises an HLA class I allele. In some embodiments, the second, inhibitor antigen comprises an allele of HLA class I that is lost in a target cell through LOH. HLA-A is a group of human leukocyte antigens (HLA) of the major histocompatibility complex (MHC) that are encoded by the HLA-A locus. HLA-A is one of three major types of human MHC class I cell surface receptors. The receptor is a heterodimer comprising a heavy a chain and smaller R chain. The a chain is encoded by a variant of HLA-A, while the R chain (02-microglobulin) is invariant. There are several thousand HLA-A variants, all of which fall within the scope of the instant disclosure.

In some embodiments, the second, inhibitor antigen comprises an HLA-B allele. The HLA-B gene has many possible variations (alleles). Hundreds of versions (alleles) of the HLA-B gene are known, each of which is given a particular number (such as HLA-B27).

In some embodiments, the second, inhibitor antigen comprises an HLA-C allele. HLA-C belongs to the HLA class I heavy chain paralogues. This class I molecule is a heterodimer consisting of a heavy chain and a light chain (beta-2 microglobulin). Over one hundred HLA-C alleles have been described.

In some embodiments, the HLA class I allele has broad or ubiquitous RNA expression.

In some embodiments, the HLA class I allele has a known, or generally high minor allele frequency.

In some embodiments, the HLA class I allele does not require a peptide-MHC antigen, for example when the HLA class I allele is recognized by a pan-HLAn antigen binding domain.

In some embodiments, the second inhibitor antigen comprises an HLA-A allele. In some embodiments the HLA-A allele comprises HLA-A*02. Various single variable domains known in the art or disclosed herein that bind to and recognize HLA-A*02 are suitable for use in embodiments. Such scFvs include, for example and without limitation, the following mouse and humanized scFv antibodies that bind HLA-A*02 in a peptide-independent way shown in Tables 2 and 3 below (complementarity determining regions underlined):

TABLE 2
HLA-A*02 ScFv Sequences and Inhibitory receptor Constructs

| Construct | Protein Sequence | DNA Sequence |
|---|---|---|
| VL-VH | MDMRVPAQLLGLLL | ATGGACATGAGGGTCCCCGCTCAGCTCCTGGGGCTCCTGCTA |
| | LWLRGARCDVLMT | CTCTGGCTCCGAGGTGCCAGATGTGATGTTTTGATGACCCAA |
| | QTPLSLPVSLGDQAS | ACTCCACTCTCCCTGCCTGTCAGTCTTGGAGATCAAGCCTCCA |
| | ISCRSSQSIVHSNGN | TCTCTTGCAGATCTAGTCAGAGCATTGTACATAGTAATGGAAA |
| | TYLEWYLQKPGQSP | CACCTATTTAGAATGGTACCTGCAGAAACCAGGCCAGTCTCCA |
| | KLLIYKVSNRFSGVP | AAGCTCCTGATCTACAAAGTTTCCAACCGATTTTCTGGGGTCC |
| | DRFSGSGSGTDFTLK | CAGACAGGTTCAGTGGCAGTGGATCAGGGACAGATTTCACAC |
| | ISRVEAEDLGVYYCF | TCAAGATCAGTAGAGTGGAGGCTGAGGATCTGGGAGTTTATT |
| | QGSHVPRTSGGGTK | ACTGCTTTCAAGGTTCACATGTTCCTCGGACGTCCGGTGGAG |
| | LEIKGGGGSGGGGS | GCACCAAGCTGGAAATCAAAGGCGGAGGTGGAAGCGGAGG |
| | GGGGSGGQVQLQQ | GGGAGGATCTGGCGGCGGAGGAAGCGGAGGCCAGGTCCAG |
| | SGPELVKPGASVRIS | CTGCAGCAGTCTGGACCTGAGCTGGTGAAGCCTGGGGCTTCA |
| | CKASGYTFTSYHIHW | GTGAGGATATCCTGCAAGGCTTCTGGCTACACCTTCACAAGTT |
| | VKQRPGQGLEWIG | ACCATATACATTGGGTGAAGCAGAGGCCTGGACAGGGACTTG |
| | WIYPGNVNTEYNEK | AGTGGATTGGATGGATTTATCCTGGAAATGTTAATACTGAGT |
| | FKGKATLTADKSSST | ACAATGAGAAGTTCAAGGGCAAGGCCACACTGACTGCAGAC |
| | AYMHLSSLTSEDSAV | AAATCGTCCAGCACAGCCTACATGCACCTCAGCAGCCTGACCT |
| | YFCAREEITYAMDY | CTGAGGACTCTGCGGTCTATTTCTGTGCCAGAGAGGAGATTA |
| | WGQGTSVTVSS | CCTATGCTATGGACTACTGGGGTCAAGGAACCTCAGTCACCG |
| | | TGTCCTCA |
| VH-VL | MDMRVPAQLLGLLL | ATGGACATGAGGGTCCCCGCTCAGCTCCTGGGGCTCCTGCTA |
| | LWLRGARCQVQLV | CTCTGGCTCCGAGGTGCCAGATGTCAGGTGCAGCTGGTGCAG |
| | QSGAEVKKPGSSVK | TCTGGGGCTGAGGTGAAGAAGCCTGGGTCCTCAGTGAAGGT |
| | VSCKASGYTFTSYHI | TTCCTGCAAGGCTTCTGGATACACCTTCACTAGCTATCATATAC |
| | HWVRQAPGQGLE | ATTGGGTGCGCCAGGCCCCCGGACAAGGGCTTGAGTGGATC |
| | WIGWIYPGNVNTEY | GGATGGATCTACCCTGGCAATGTTAACACAGAATATAATGAG |
| | NEKFKGKATITADES | AAGTTCAAGGGCAAAGCCACCATTACCGCGGACGAATCCACG |
| | TNTAYMELSSLRSED | AACACAGCCTACATGGAGCTGAGCAGCCTGAGATCTGAAGAC |
| | TAVYYCAREEITYAM | ACGGCTGTGTATTACTGTGCGAGGGAGGAAATTACCTACGCT |
| | DYWGQGTLVTVSSG | ATGGACTACTGGGGCCAGGGAACCCTGGTCACCGTGTCCTCA |
| | GGGSGGGGSGGGG | GGCGGAGGTGGAAGCGGAGGGGGAGGATCTGGCGGCGGAG |
| | SGGDIQMTQSPSTL | GAAGCGGAGGCGACATTCAAATGACCCAGAGCCCATCCACCC |
| | SASVGDRVTITCRSS | TGAGCGCATCTGTAGGTGACCGGGTCACCATCACTTGTAGAT |
| | QSIVHSNGNTYLEW | CCAGTCAGAGTATTGTACACAGTAATGGGAACACCTATTTGG |
| | YQQKPGKAPKLLIYK | AATGGTATCAGCAGAAACCAGGTAAAGCCCCAAAATTGCTCA |
| | VSNRFSGVPARFSGS | TCTACAAAGTCTCTAACAGATTTAGTGGTGTACCAGCCAGGTT |
| | GSGTEFTLTISSLQPD | CAGCGGTTCCGGAAGTGGTACTGAATTCACCCTCACGATCTCC |
| | DFATYYCFQGSHVP | TCTCTCCAGCCAGATGATTTCGCCACTTATTACTGTTTTCAAGG |
| | RTFGQGTKVEVK | TTCACATGTGCCGCGCACATTCGGTCAGGGTACTAAAGTAGA |
| | | AGTCAAA |
| VH-VL | MDMRVPAQLLGLLL | ATGGACATGAGGGTCCCCGCTCAGCTCCTGGGGCTCCTGCTA |
| | LWLRGARCQVQLV | CTCTGGCTCCGAGGTGCCAGATGTCAGGTGCAGCTGGTGCAG |
| | QSGAEVKKPGSSVK | TCTGGGGCTGAGGTGAAGAAGCCTGGGTCCTCAGTGAAGGT |
| | VSCKASGYTFTSYH | TTCCTGCAAGGCTTCTGGATACACCTTCACTAGCTATCATATG |
| | MHWVRQAPGQGL | CATTGGGTGCGCCAGGCCCCCGGACAAGGGCTTGAGTGGAT |
| | EWIGYIYPGNVNTEY | CGGATACATCTACCCTGGCAATGTTAACACAGAATATAATGA |
| | NEKFKGKATLTADKS | GAAGTTCAAGGGCAAAGCCACCCTTACCGCGGACAAATCCAC |
| | TNTAYMELSSLRSED | GAACACAGCCTACATGGAGCTGAGCAGCCTGAGATCTGAAGA |
| | TAVYFCAREEITYAM | CACGGCTGTGTATTTCTGTGCGAGGGAGGAAATTACCTACGC |
| | DYWGQGTLVTVSSG | TATGGACTACTGGGGCCAGGGAACCCTGGTCACCGTGTCCTC |
| | GGGSGGGGSGGGG | AGGCGGAGGTGGAAGCGGAGGGGGAGGATCTGGCGGCGGA |
| | SGGDVQMTQSPSTL | GGAAGCGGAGGCGACGTTCAAATGACCCAGAGCCCATCCACC |
| | SASVGDRVTITCSSS | CTGAGCGCATCTGTAGGTGACCGGGTCACCATCACTTGTAGC |
| | QSIVHSNGNTYME | TCCAGTCAGAGTATTGTACACAGTAATGGGAACACCTATATG |
| | WYQQKPGKAPKLLI | GAATGGTATCAGCAGAAACCAGGTAAAGCCCCAAAATTGCTC |
| | YKVSNRFSGVPDRFS | ATCTACAAAGTCTCTAACAGATTTAGTGGTGTACCAGACAGGT |
| | GSGSGTEFTLTISSLQ | TCAGCGGTTCCGGAAGTGGTACTGAATTCACCCTCACGATCTC |
| | PDDFATYYCHQGSH | CTCTCTCCAGCCAGATGATTTCGCCACTTATTACTGTCATCAAG |
| | VPRTFGQGTKVEVK | GTTCACATGTGCCGCGCACATTCGGTCAGGGTACTAAAGTAG |
| | | AAGTCAAA |

In some embodiments, the second antigen binding domain comprises a scFv domain that binds to HLA-A*02 antigen. In some embodiments, the scFv domain comprises a sequence of disclosed in Table 2, or a sequence having at least 90%, at least 95%, at least 97%, at least 99% or is identical thereto.

TABLE 3
Additional HLA-A*02 scFv binding domains

mouse:
DVLMTQTPLSLPVSLGDQASISCRSSQSIVHSNGNTYLEWYLQKPGQSP
KLLIYKVSNRFSGVPDRFSGSGSGTDFTLKISRVEAEDLGVYYCFQGSH
VPRTSGGGTKLEIKGGGGSGGGGSGGGGSGGQVQLQQSGPELVKPGASV
RISCKASGYTFTSYHIHWVKQRPGQGLEWIGWIYPGNVNTEYNEKFKGK
ATLTADKSSSTAYMHLSSLTSEDSAVYFCAREEITYAMDYWGQGTSVTV
SS
humanized:
QVQLVQSGAEVKKPGSSVKVSCKASGYTFTSYHIHWVRQAPGQGLEWMG
WIYPGNVNTEYNEKFKGKATITADKSTSTAYMELSSLRSEDTAVYYCAR
EEITYAMDYWGQGTTVTVSSGGGGSGGGGSGGGGSGGEIVLTQSPGTLS
LSPGERATLSCRSSQSIVHSNGNTYLEWYQQKPGQAPRLLIYKVSNRFS
GIPDRFSGSGSGTDFTLTISRLEPEDFAVYYCFQGSHVPRTFGGGTKVE
IK
humanized:
QVQLVQSGAEVKKPGSSVKVSCKASGYTFTSYHIHWVRQAPGQGLEWMG
WIYPGNVNTEYNEKFKGKATITADKSTSTAYMELSSLRSEDTAVYYCAR
EEITYAMDYWGQGTTVTVSSGGGGSGGGGSGGGGSGGDIVMTQTPLSLP
VTPGEPASISCRSSQSIVHSNGNTYLEWYLQKPGQSPQLLIYKVSNRFS
GVPDRFSGSGSGTDFTLKISRVEAEDVGVYYCFQGSHVPRTFGGGTKVE
IK
humanized:
EVQLVESGGGLVKPGGSLRLSCAASGYTFTSYHIHWVRQAPGKGLEWVG
WIYPGNVNTEYNEKFKGRFTISRDDSKNTLYLQMNSLKTEDTAVYYCAR
EEITYAMDYWGQGTTVTVSSGGGGSGGGGSGGGGSGGDIQMTQSPSSLS
ASVGDRVTITCRSSQSIVHSNGNTYLEWYQQKPGKAPKLLIYKVSNRFS
GVPSRFSGSGSGTDFTLTISSLQPEDFATYYCFQGSHVPRTFGGGTKVE
IK
humanized:
QVQLVQSGAEVKKPGSSVKVSCKASGYTFTSYHIHWVRQAPGQGLEWIG
WIYPGNVNTEYNEKFKGKATITADESTNTAYMELSSLRSEDTAVYYCAR
EEITYAMDYWGQGTLVTVSSGGGGSGGGGSGGGGSGGDIQMTQSPSTLS
ASVGDRVTITCRSSQSIVHSNGNTYLEWYQQKPGKAPKLLIYKVSNRFS
GVPARFSGSGSGTEFTLTISSLQPDDFATYYCFQGSHVPRTFGQGTKVE
VK
humanized:
QVQLVQSGAEVKKPGSSVKVSCKASGYTFTSYHMHWVRQAPGQGLEWIG
YIYPGNVNTEYNEKFKGKATLTADKSTNTAYMELSSLRSEDTAVYFCAR
EEITYAMDYWGQGTLVTVSSGGGGSGGGGSGGGGSGGDVQMTQSPSTLS
ASVGDRVTITCSSSQSIVHSNGNTYMEWYQQKPGKAPKLLIYKVSNRFS
GVPDRFSGSGSGTEFTLTISSLQPDDFATYYCHQGSHVPRTFGQGTKVE
VK
mouse:
QVQLQQSGPELVKPGASVKMSCKASGYTFTSYHIQWVKQRPGQGLEWIG
WIYPGDGSTQYNEKFKGKTTLTADKSSSTAYMLLSSLTSEDSAIYFCAR
EGTYYAMDYWGQGTSVTVSSGGGGSGGGGSGGGGSGGDVLMTQTPLSLP
VSLGDQVSISCRSSQSIVHSNGNTYLEWYLQKPGQSPKLLIYKVSNRFS
GVPDRFSGSGSGTDFTLKISRVEAEDLGVYYCFQGSHVPRTFGGGTKLE
IK
humanized:
QLQLQESGPGLVKPSETLSLTCTVSGYTFTSYHIQWIRQPPGKGLEWIG
WIYPGDGSTQYNEKFKGRATISVDTSKNQFSLNLDSVSAADTAIYYCAR
EGTYYAMDYWGKGSTVTVSSGGGGSGGGGSGGGGSGGDIQMTQSPSSLS
ASVGDRVTITCRSSQSIVHSNGNTYLEWYQQKPGKAPKLLIYKVSNRFS
GVPSRFSGSGSGTDFTFTISSLQPEDIATYYCFQGSHVPRTFGPGTKVD
IK
humanized:
EVQLVQSGAELKKPGSSVKVSCKASGYTFTSYHIQWVKQAPGQGLEWIG
WIYPGDGSTQYNEKFKGKATLTVDKSTNTAYMELSSLRSEDTAVYYCAR
EGTYYAMDYWGQGTLVTVSSGGGGSGGGGSGGGGSGGDIQMTQSPSTLS
ASVGDRVTITCRSSQSIVHSNGNTYLEWYQQKPGKAPKLLIYKVSNRFS
GVPSRFSGSGSGTDFTLTISSLQPDDFATYYCFQGSHVPRTFGQGTKVE
VK
humanized:
QVQLVQSGAEVKKPGSSVKVSCKASGYTFTSYHIQWVRQAPGQGLEWMG
WIYPGDGSTQYNEKFKGRVTITADKSTSTAYMELSSLRSEDTAVYYCAR
EGTYYAMDYWGQGTTVTVSSGGGGSGGGGSGGGGSGGEIVLTQSPGTLS
LSPGERATLSCRSSQSIVHSNGNTYLEWYQQKPGQAPRLLIYKVSNRFS
GIPDRFSGSGSGTDFTLTISRLEPEDFAVYYCFQGSHVPRTFGGGTKVE
IK
humanized:
QVTLKQSGAEVKKPGSSVKVSCTASGYTFTSYHVSWVRQAPGQGLEWLG
RIYPGDGSTQYNEKFKGKVTITADKSMDTSFMELTSLTSEDTAVYYCAR
EGTYYAMDLWGQGTLVTVSSGGGGSGGGGSGGGGSGGEIVLTQSPGTLS
LSPGERATLSCRSSQSIVHSNGNTYLAWYQQKPGQAPRLLISKVSNRFS
GVPDRFSGSGSGTDFTLTISRLEPEDFAVYYCQQGSHVPRTFGGGTKVE
IK
humanized:
QVQLVQSGAEVKKPGASVKVSCKASGYTFTSYHMHWVRQAPGQRLEWMG
WIYPGDGSTQYNEKFKGKVTITRDTSASTAYMELSSLRSEDTAVYYCAR
EGTYYAMDYWGQGTLVTVSSGGGGSGGGGSGGGGSGGDIVMTQTPLSLP
VTPGEPASISCRSSQSIVHSNGNTYLDWYLQKPGQSPQLLIYKVSNRFS
GVPDRFSGSGSGTDFTLKISRVEAEDVGVYYCMQGSHVPRTFGGGTKVE
IK

Exemplary heavy chain and light chain CDRs (CDR-H1, CDR-H-2 and CDR-H3, or CDR-L1, CDR-L2 and CDR-L3, respectively) for HLA-A*02 antigen binding domains are shown in Table 4 below.

TABLE 4
HLA-A*02 CDR Sequences

| CDR-L1 | CDR-L2 | CDR-L3 | CDR-H1 | CDR-H2 | CDR-H3 |
|---|---|---|---|---|---|
| RSSQSIV | KVSNRFS | FQGSHVP | ASGYTFT | WIYPGNVN | EEITYAMD |
| HSNGNT | GVPDR | RT | SYHIH | TEYNEKFK | Y |
| YLE | | | | GK | |
| RSSQSIV | KVSNRFS | FQGSHVP | ASGYTFT | WIYPGNVN | EEITYAMD |
| HSNGNT | GVPAR | RT | SYHIH | TEYNEKFK | Y |
| YLE | | | | GK | |
| SSSQSIV | KVSNRFSGV | HQGSHVP | ASGYTFT | YIYPGNVN | EEITYAMD |
| HSNGNTY | PDR | RT | SYHMH | TEYNEKFK | Y |
| ME | | | | GK | |
| RSSQSIV | KVSNRFS | MQGSHVP | SGYTFTS | WIYPGDGS | EGTYYAMD |
| HSNGNT | GVPDR | RT | YHMH | TQYNEKFK | Y |
| YLD | | | | G | |

In some embodiments, the scFv comprises the complementarity determined regions (CDRs) disclosed in Table 3.

In some embodiments, the inhibitor antigen is HLA-A*02, and the inhibitory antigen binding domain comprises an HLA-A*02 antigen binding domain. In some embodiments, the inhibitory receptor antigen binding domain binds HLA-A*02 independent of the peptide in a pMHC complex comprising HLA-A*02. In some embodiments, the HLA-A*02 antigen binding domain comprises a scFv domain.

In some embodiments, the inhibitor antigen comprises a minor histocompatibility antigen (MiHA). In some embodiments, the second, inhibitor antigen comprises an allele of a MiHA that is lost in a target cell through LOH.

MiHAs are peptides derived from proteins that contain nonsynonymous differences between alleles and are displayed by common HLA alleles. The non-synonymous differences can arise from SNPs, deletions, frameshift mutations or insertions in the coding sequence of the gene encoding the MiHA. Exemplary MiHAs can be about 9-12 amino acids in length and can bind to MHC class I and MHC class II proteins. Binding of the TCR to the MHC complex displaying the MiHA can activate T cells. The genetic and immunological properties of MiHAs will be known to the person of ordinary skill in the art. Candidate MiHAs are known peptides presented by known HLA class I alleles, are known to elicit T cell responses in the clinic (for example, in graft versus host disease, or transplant rejection, and allow for patient selection by simple SNP genotyping.

In some embodiments, the MiHA has broad or ubiquitous RNA expression.

In some embodiments, the MiHA has high minor allele frequency.

In some embodiments, the MiHA comprises a peptide derived from a Y chromosome gene.

In some embodiments, the second, inhibitor antigen comprises a Y chromosome gene, i.e. peptide encoded by a gene on the Y chromosome. In some embodiments, the second, inhibitor antigen comprises a peptide encoded by a Y chromosome gene that is lost in target cells through loss of Y chromosome (LoY). For example, about a third of the characterized MiHAs come from the Y chromosome. The Y chromosome contains over 200 protein coding genes, all of which are envisaged as within the scope of the instant disclosure.

As used herein, “loss of Y”, or “LoY” refers a genetic change that occurs at high frequency in tumors whereby one copy of part or all of the Y chromosome is deleted, leading to a loss of Y chromosome encoded gene(s). Loss of Y chromosome is known to occur in certain cancers. For example, there is a reported 40% somatic loss of Y chromosome in renal clear cell cancers (Arseneault et al., Sci. Rep. 7: 44876 (2017)). Similarly, clonal loss of the Y chromosome was reported in 5 out of 31 in male breast cancer subjects (Wong et al., Oncotarget 6(42):44927-40 (2015)). Loss of the Y chromosome in tumors from male patients has been described as a “consistent feature” of head and neck cancer patients (el-Naggar et al., Am J Clin Pathol 105(1):102-8 (1996)). Further, Y chromosome loss was associated with X chromosome disomy in four of seven male patients with gastric cancer (Saal et al., Virchows Arch B Cell Pathol (1993)). Thus, Y chromosome genes can be lost in a variety of cancers, and can be used as inhibitor antigens with the engineered receptors of the instant disclosure targeting cancer cells.

In some embodiments, the inhibitory receptor comprises a hinge domain. In some embodiments, the hinge is isolated or derived from CD8α or CD28. In some embodiments, the CD8α hinge comprises an amino acid sequence having at least 80% identity, at least 90% identity, at least 95% identity, at least 99% identity or is identical to a sequence of TTTPAPRPPTPAPTIASQPLSLRPEACRPAAGGAVHTRGLDFACD. In some embodiments, the CD28 hinge comprises an amino acid sequence having at least 80% identity, at least 90% identity, at least 95% identity, at least 99% identity or is identical to a sequence of CTIEVMYPPPYLDNEKSNGTIIHVKGKHLCPSPLFPGPSKP.

In some embodiments the inhibitory receptor comprises a CD28 transmembrane domain. In some embodiments, the CD28 transmembrane domain comprises an amino acid sequence having at least 90% identity, at least 95% identity, at least 99% identity or is identical to a sequence of FWVLVVVGGVLACYSLLVTVAFIIFWV.

In some embodiments, the inhibitory receptor comprises a transmembrane domain isolated or derived from CD8α. In some embodiments, the CD8α transmembrane domain comprises an amino acid sequence having at least 90% identity, at least 95% identity, at least 99% identity or is identical to a sequence of IYIWAPLAGTCGVLLLSLVIT.

In some embodiments of the inhibitor receptors of the disclosure, the inhibitory signal is transmitted through the intracellular domain of the receptor. In some embodiments, the inhibitory receptor comprises an inhibitory intracellular domain. In some embodiments, the inhibitory receptor is a CAR comprising an inhibitory intracellular domain (an inhibitory CAR). In some embodiments, the inhibitory receptor is a TCR comprising an inhibitory intracellular domain (an inhibitory TCR).

In some embodiments, the inhibitory intracellular domain comprises an immunoreceptor tyrosine-based inhibitory motif (ITIM). In some embodiments, the inhibitory intracellular domain comprising an ITIM can be isolated or derived from an immune checkpoint inhibitor such as CTLA-4 and PD-1. CTLA-4 and PD-1 are immune inhibitory receptors expressed on the surface of T cells, and play a pivotal role in attenuating or terminating T cell responses.

Inhibitory domains can be isolated from human tumor necrosis factor related apoptosis inducing antigen (TRAIL) receptor and CD200 receptor 1.

In some embodiments, the inhibitory domain comprises an intracellular domain, a transmembrane or a combination thereof. In some embodiments, the inhibitory domain comprises an intracellular domain, a transmembrane domain, a hinge region or a combination thereof. In some embodiments, the inhibitory domain comprises an immunoreceptor tyrosine-based inhibitory motif (ITIM). In some embodiments, the inhibitory domain comprising an ITIM can be isolated or derived from an immune checkpoint inhibitor such as CTLA-4 and PD-1.

Inhibitory domains can be isolated from human tumor necrosis factor related apoptosis inducing antigen (TRAIL) receptor and CD200 receptor 1. In some embodiments, the inhibitory domain is isolated or derived from a human protein, for example a human TRAIL receptor, CTLA-4, or PD-1 protein. In some embodiments, the TRAIL receptor comprises TR10A, TR10B or TR10D.

Endogenous TRAIL is expressed as a 281-amino acid type II trans-membrane protein, which is anchored to the plasma membrane and presented on the cell surface. TRAIL is expressed by natural killer cells, which, following the establishment of cell-cell contacts, can induce TRAIL-dependent apoptosis in target cells. Physiologically, the TRAIL-signaling system was shown to be essential for immune surveillance, for shaping the immune system through regulating T-helper cell 1 versus T-helper cell 2 as well as “helpless” CD8+ T-cell numbers, and for the suppression of spontaneous tumor formation.

In some embodiments, the inhibitory domain comprises an intracellular domain isolated or derived from a CD200 receptor. The cell surface glycoprotein CD200 receptor 1 (Uniprot ref. Q8TD46) represents another example of an inhibitory intracellular domain of the present invention. This inhibitory receptor for the CD200/OX2 cell surface glycoprotein limits inflammation by inhibiting the expression of proinflammatory molecules including TNF-alpha, interferons, and inducible nitric oxide synthase (iNOS) in response to selected stimuli.

In some embodiments, the engineered receptor comprises an inhibitory domain isolated or derived from killer cell immunoglobulin like receptor, three Ig domains and long cytoplasmic tail 2 (KIR3DL2), killer cell immunoglobulin like receptor, three Ig domains and long cytoplasmic tail 3 (KIR3DL3), leukocyte immunoglobulin like receptor B1 (LIR1, also called LIR-1 and LILRB1), programmed cell death 1 (PD-1), Fc gamma receptor IIB (FcgRIIB), killer cell lectin like receptor KI (NKG2D), CTLA-4, a domain containing a synthetic consensus ITIM, a ZAP70 SH2 domain (e.g., one or both of the N and C terminal SH2 domains), or ZAP70 KI_K369A (kinase inactive ZAP70).

In some embodiments, the inhibitory domain is isolated or derived from a human protein.

In some embodiments, the second, inhibitory receptor comprises a cytoplasmic domain and transmembrane domain isolated or derived from the same protein, for example an ITIM containing protein. In some embodiments, the second, inhibitory receptor comprises a cytoplasmic domain, a transmembrane domain, and an extracellular domain or a portion thereof isolated or derived isolated or derived from the same protein, for example an ITIM containing protein. In some embodiments, the second, inhibitory receptor comprises a hinge region isolated or derived from isolated or derived from the same protein as the intracellular domain and/or transmembrane domain, for example an ITIM containing protein. In some embodiments, the second, inhibitory receptor comprises an inhibitory domain. In some embodiments, the second, inhibitory receptor comprises an inhibitory intracellular domain and/or an inhibitory transmembrane domain. In some embodiments, the second engineered receptor is a CAR comprising an inhibitory domain (an inhibitory CAR). In some embodiments, the inhibitory intracellular domain is fused to the intracellular domain of the CAR. In some embodiments, the inhibitory intracellular domain is fused to the transmembrane domain of a CAR. In some embodiments, the inhibitory intracellular domain is fused to the intracellular domain of a TCR subunit. In some embodiments, the inhibitory intracellular domain is fused to the transmembrane domain of a TCR subunit.

The present disclosure describes inhibitory receptors having one or more domains from Leukocyte immunoglobulin-like receptor subfamily B member 1 (LILRB1, or LIR1). In some embodiments, the inhibitory receptor comprises a LILRB1 intracellular domain or a functional variant thereof.

Leukocyte immunoglobulin-like receptor subfamily B member 1 (LILRB1), also known as Leukocyte immunoglobulin-like receptor B1, as well as ILT2, LIR1, MIR7, PIRB, CD85J, ILT-2 LIR-1, MIR-7 and PIR-B, is a member of the leukocyte immunoglobulin-like receptor (LIR) family. The LILRB1 protein belongs to the subfamily B class of LIR receptors. These receptors contain two to four extracellular immunoglobulin domains, a transmembrane domain, and two to four cytoplasmic immunoreceptor tyrosine-based inhibitory motifs (ITIMs). The LILRB1 receptor is expressed on immune cells, where it binds to MHC class I molecules on antigen-presenting cells and transduces a negative signal that inhibits stimulation of an immune response. LILRB1 is thought to regulate inflammatory responses, as well as cytotoxicity, and to play a role in limiting auto-reactivity. Multiple transcript variants encoding different isoforms of LILRB1 exist, all of which are contemplated as within the scope of the instant disclosure.

In various embodiments, a chimeric antigen receptor is provided, comprising a polypeptide, wherein the polypeptide comprises one or more of: an LILRB1 hinge domain or functional fragment or variant thereof; an LILRB1 transmembrane domain or a functional variant thereof; and an LILRB1 intracellular domain or an intracellular domain comprising at least one, or at least two immunoreceptor tyrosine-based inhibitory motifs (ITIMs), wherein each ITIM is independently selected from NLYAAV, VTYAEV, VTYAQL, and SIYATL.

In some embodiments, the inhibitory receptor comprises a LILRB1 intracellular domain or a functional variant thereof, the sequences of which are described in Table 5 below.

As used herein an “immunoreceptor tyrosine-based inhibitory motif” or “ITIM” refers to a conserved sequence of amino acids with a consensus sequence of S/I/V/LxYxxI/V/L, or the like, that is found in the cytoplasmic tails of many inhibitory receptors of the immune system. After ITIM-possessing inhibitory receptors interact with their antigen, the ITIM motif is phosphorylated, allowing the inhibitory receptor to recruit other enzymes, such as the phosphotyrosine phosphatases SHP-1 and SHP-2, or the inositol-phosphatase called SHIP.

In some embodiments, the polypeptide comprises an intracellular domain comprising at least one immunoreceptor tyrosine-based inhibitory motif (ITIM), at least two ITIMs, at least 3 ITIMs, at least 4 ITIMs, at least 5 ITIMs or at least 6 ITIMs. In some embodiments, the intracellular domain has 1, 2, 3, 4, 5, or 6 ITIMs.

In some embodiments, the polypeptide comprises an intracellular domain comprising at least one ITIM, or at least two ITIMs, at least 3 ITIMs, at least 4 ITIMs or at least 5 ITIMs independently selected from the group of ITIMs consisting of NLYAAV, VTYAEV, VTYAQL, and SIYATL.

In some embodiments, the inhibitory receptor comprises a transmembrane domain.

A “transmembrane domain”, as used herein, refers to a domain of a protein that spans membrane of the cell. Transmembrane domains typically consist predominantly of non-polar amino acids, and may traverse the lipid bilayer once or several times. Transmembrane domains usually comprise alpha helices, a configuration which maximizes internal hydrogen bonding.

Transmembrane domains of the inhibitory receptors of the disclosure may be isolated or derived from any source are envisaged as within the scope of the fusion proteins of the disclosure. The transmembrane domain may be derived either from a natural or from a recombinant source. Where the source is natural, the domain may be derived from any membrane-bound or transmembrane protein. Exemplary transmembrane domains may include at least the transmembrane region(s) of e.g., the alpha, beta or zeta chain of the TCR, CD3 delta, CD3 epsilon or CD3 gamma, CD28, CD3 epsilon, CD45, CD4, CD5, CD8, CD9, CD16, CD22, CD33, CD37, CD64, CD80, CD86, CD134, CD137, CD154.

In some embodiments, the inhibitory receptor comprises a LILRB1 transmembrane domain, or a functional variant thereof, the sequences of which are described in Table 5 below.

In some embodiments, the transmembrane comprises a TCR alpha transmembrane domain. In some embodiments, the TCR alpha transmembrane domain comprises an amino acid sequence having at least 85% identity, at least 90% identity, at least 95% identity, at least 96% identity, at least 97% identity, at least 98% identity, at least 99% identity or is identical to a sequence of: VIGFRILLLKVAGFNLLMTLRLW.

In some embodiments, the transmembrane comprises a TCR beta transmembrane domain. In some embodiments, the TCR beta transmembrane domain comprises an amino acid sequence having at least 85 identity, at least 90% identity, at least 95% identity, at least 96% identity, at least 97% identity, at least 98% identity, at least 99% identity or is identical to a sequence of: TILYEILLGKATLYAVLVSALVL.

In some embodiments the transmembrane domain comprise a CD8α or CD28 transmembrane domain, as described herein for the activator receptor.

In some embodiments, the transmembrane domain can be attached to the extracellular region chimeric antigen receptor, e.g., the antigen-binding domain or antigen binding domain, via a hinge, e.g., a hinge from a human protein. For example, in some embodiments, the hinge can be a human immunoglobulin (Ig) hinge, e.g., an IgG4 hinge, a CD8α hinge or an LILRB1 hinge as described above for the activator receptor. In some embodiments, the inhibitory receptor comprises a LILRB1 hinge domain, or a functional variant thereof, the sequences of which are described in Table 5 below.

Additional sequences for the LILRB1 based inhibitory receptors of the disclosure are shown in Table 5 below.

TABLE 5
Polypeptide Sequences for Elements of
Illustrative Inhibitory Receptors

| Name | Sequence |
|---|---|
| LILRB1 | MTPILTVLICLGLSLGPRTHVQAGHLPKPTLWAEPGSVITQ |
| | GSPVTLRCQGGQETQEYRLYREKKTALWITRIPQELVKKG |
| | QFPIPSITWEHAGRYRCYYGSDTAGRSESSDPLELVVTGAY |
| | IKPTLSAQPSPVVNSGGNVILQCDSQVAFDGFSLCKEGEDE |
| | HPQCLNSQPHARGSSRAIFSVGPVSPSRRWWYRCYAYDSN |
| | SPYEWSLPSDLLELLVLGVSKKPSLSVQPGPIVAPEETLTL |
| | QCGSDAGYNRFVLYKDGERDFLQLAGAQPQAGLSQANFT |
| | LGPVSRSYGGQYRCYGAHNLSSEWSAPSDPLDILIAGQFY |
| | DRVSLSVQPGPTVASGENVTLLCQSQGWMQTFLLTKEGA |
| | ADDPWRLRSTYQSQKYQAEFPMGPVTSAHAGTYRCYGS |
| | QSSKPYLLTHPSDPLELVVSGPSGGPSSPTTGPTSTSGPED |
| | QPLTPTGSDPQSGLGRHLGVVIGILVAVILLLLLLLLLFLILRH |
| | RRQGKHWTSTQRKADFQHPAGAVGPEPTDRGLQWRS |
| | SPAADAQEENLYAAVKHTQPEDGVEMDTRSPHDEDPQAV |
| | TYAEVKHSRPRREMASPPSPLSGEFLDTKDRQAEEDRQM |
| | DTEAAASEAPQDVTYAQLHSLTLRREATEPPPSQEGPSPA |
| | VPSIYATLAIHPSQEGPSPAVPSIYATLAIH |
| LILRB1 hinge- | YGSQSSKPYLLTHPSDPLELVVSGPSGGPSSPTTGPTSTSG |
| transmembrane- | PEDQPLTPTGSDPQSGLGRHLGVVIGILVAVILLLLLLLLL |
| intracellular | FLILRHRRQGKHWTSTQRKADFQHPAGAVGPEPTDRGLQ |
| domain | WRSSPAADAQEENLYAAVKHTQPEDGVEMDTRSPHDEDP |
| | QAVTYAEVKHSRPRREMASPPSPLSGEFLDTKDRQAEEDR |
| | QMDTEAAASEAPQDVTYAQLHSLTLRREATEPPPSQEGPS |
| | PAVPSIYATLAIH |
| LILRB1 hinge- | VVSGPSGGPSSPTTGPTSTSGPEDQPLTPTGSDPQSGLGRH |
| transmembrane- | LGVVIGILVAVILLLLLLLLLFLILRHRRQGKHWTSTQRK |
| intracellular | ADFQHPAGAVGPEPTDRGLQWRSSPAADAQEENLYAAVK |
| domain (w/o | HTQPEDGVEMDTRSPHDEDPQAVTYAEVKHSRPRREMAS |
| YGSQSSKPYLL | PPSPLSGEFLDTKDRQAEEDRQMDTEAAASEAPQDVTYA |
| THPSDPLEL) | QLHSLTLRREATEPPPSQEGPSPAVPSIYATLAIH |
| LILRB1 hinge | YGSQSSKPYLLTHPSDPLELVVSGPSGGPSSPTTGPTSTSG |
| domain | PEDQPLTPTGSDPQSGLGRHLG |
| LILRB1 | VVIGILVAVILLLLLLLLLFLIL |
| transmembrane | |
| domain | |
| LILRB1 | RHRRQGKHWTSTQRKADFQHPAGAVGPEPTDRGLQWRS |
| intracellular | SPAADAQEENLYAAVKHTQPEDGVEMDTRSPHDEDPQAV |
| domain | TYAEVKHSRPRREMASPPSPLSGEFLDTKDRQAEEDRQMD |
| | TEAAASEAPQDVTYAQLHSLTLRREATEPPPSQEGPSPAVP |
| | SIYATLAIH |
| ITIM1 | NLYAAV |
| ITIM2 | VTYAEV |
| ITIM3 | VTYAQL |
| ITIM4 | SIYATL |
| ITIM1-2 | NLYAAVKHTQPEDGVEMDTRSPHDEDPQAVTYAEV |
| ITIM2-3 | VTYAEVKHSRPRREMASPPSPLSGEFLDTKDRQAEEDRQ |
| | MDTEAAASEAPQDVTYAQL |
| ITIM3-4 | VTYAQLHSLTLRREATEPPPSQEGPSPAVPSIYATL |
| ITIM1-3 | NLYAAVKHTQPEDGVEMDTRSPHDEDPQAVTYAEVKHS |
| | RPRREMASPPSPLSGEFLDTKDRQAEEDRQMDTEAAASEA |
| | PQDVTYAQL |
| ITIM2-4 | VTYAEVKHSRPRREMASPPSPLSGEFLDTKDRQAEEDRQ |
| | MDTEAAASEAPQDVTYAQLHSLTLRREATEPPPSQEGPSP |
| | AVPSIYATL |
| ITIM1-4 | NLYAAVKHTQPEDGVEMDTRSPHDEDPQAVTYAEVKHS |
| | RPRREMASPPSPLSGEFLDTKDRQAEEDRQMDTEAAASEA |
| | PQDVTYAQLHSLTLRREATEPPPSQEGPSPAVPSIYATL |
| D3D4 domain | YGSQSSKPYLLTHPSDPLEL |
| Short hinge | VVSGPSGGPSSPTTGPTSTSGPEDQPLTPTGSDPQSGLGRH |
| | LG |
| Hinge (iTIM | YGSQSSKPYLLTHPSDPLELVVSGPSGGPSSPTTGPTSTSGP |
| hinge) | EDQPLTPTGSDPQSGLGRHLGV |
| Short hinge 2 | VVSGPSGGPSSPTTGPTSTSGPEDQPLTPTGSDPQSGLGRH |
| | LGV |
| Long hinge 1 | AGSGGSGGSGGSPVPSTPPTPSPSTPPTPSPSGGSGNSSGSG |
| | GSPVPSTPPTPSPSTPPTPSPSASV |
| Long hinge 2 | AGSGGSGGSGGSPVPSTPPTNSSSTPPTPSPSPVPSTPPTNSSST |
| | PPTPSPSPVPSTPPTNSSSTPPTPSPSASV |
| 2X short hinge | VVSGPSGGPSSPTTGPTSTSGPEDQPLTPTGSDPQSGLGRH |
| | VVSGPSGGPSSPTTGPTSTSGPEDQPLTPTGSDPQSGLGRH |
| | LGV |
| Hinge (truncated) | TTGPTSTSGPEDQPLTPTGSDPQSGLGRHLGV |
| Hinge- | YGSQSSKPYLLTHPSDPLELVVSGPSGGPSSPTTGPTSTSG |
| transmembrane | PEDQPLTPTGSDPQSGLGRHLGVVIGILVAVILLLLLLLLL |
| | FLIL |
| Transmembrane- | VVIGILVAVILLLLLLLLLFLILRHRRQGKHWTSTQRKAD |
| intracellular | FQHPAGAVGPEPTDRGLQWRSSPAADAQEENLYAAVKHT |
| domain | QPEDGVEMDTRSPHDEDPQAVTYAEVKHSRPRREMASPP |
| | SPLSGEFLDTKDRQAEEDRQMDTEAAASEAPQDVTYAQL |
| | HSLTLRREATEPPPSQEGPSPAVPSIYATLAIH |

In some embodiments, the inhibitory receptors of the disclosure comprise a more than one LILRB1 domains or functional equivalents thereof. For example, in some embodiments, the inhibitory comprises an LILRB1 hinge domain, transmembrane domain, and intracellular domain, the sequences of which are set forth in Table 6 below. Table 6. Exemplary Inhibitory receptor Hinge, Transmembrane and Intracellular Domains

| Protein Sequence | DNA Sequence |
|---|---|
| YGSQSSKPYLLTHPSD | TACGGCTCACAGAGCTCCAAACCCTACCTGCTGACTCACCCCAGTGACCCCC |
| PLELVVSGPSGGPSSP | TGGAGCTCGTGGTCTCAGGACCGTCTGGGGGCCCCAGCTCCCCGACAACAG |
| TTGPTSTSGPEDQPLT | GCCCCACCTCCACATCTGGCCCTGAGGACCAGCCCCTCACCCCCACCGGGTC |
| PTGSDPQSGLGRHLG | GGATCCCCAGAGTGGTCTGGGAAGGCACCTGGGGGTTGTGATCGGCATCT |
| VVIGILVAVILLLLLLLL | TGGTGGCCGTCATCCTACTGCTCCTCCTCCTCCTCCTCCTCTTCCTCATCCTCC |
| LFLILRHRRQGKHWT | GACATCGACGTCAGGGCAAACACTGGACATCGACCCAGAGAAAGGCTGAT |
| STQRKADFQHPAGA | TTCCAACATCCTGCAGGGGCTGTGGGGCCAGAGCCCACAGACAGAGGCCT |
| VGPEPTDRGLQWRS | GCAGTGGAGGTCCAGCCCAGCTGCCGATGCCCAGGAAGAAAACCTCTATG |
| SPAADAQEENLYAAV | CTGCCGTGAAGCACACACAGCCTGAGGATGGGGTGGAGATGGACACTCGG |
| KHTQPEDGVEMDTR | AGCCCACACGATGAAGACCCCCAGGCAGTGACGTATGCCGAGGTGAAACA |
| SPHDEDPQAVTYAEV | CTCCAGACCTAGGAGAGAAATGGCCTCTCCTCCTTCCCCACTGTCTGGGGA |
| KHSRPRREMASPPSP | ATTCCTGGACACAAAGGACAGACAGGCGGAAGAGGACAGGCAGATGGAC |
| LSGEFLDTKDRQAEE | ACTGAGGCTGCTGCATCTGAAGCCCCCCAGGATGTGACCTACGCCCAGCTG |
| DRQMDTEAAASEAP | CACAGCTTGACCCTCAGACGGGAGGCAACTGAGCCTCCTCCATCCCAGGAA |
| QDVTYAQLHSLTLRR | GGGCCCTCTCCAGCTGTGCCCAGCATCTACGCCACTCTGGCCATCCACTAG |
| EATEPPPSQEGPSPA | |
| VPSIYATLAIH | |

### Vectors and Polynucleotides

The disclosure also provides dual vector systems, comprising polynucleotides encoding the activator and inhibitory receptors described herein.

The polynucleotide, used interchangeably with nucleic acid, includes DNA and RNA such as genomic DNA, cDNA and mRNA, or combinations thereof. The nucleic acid may comprise, in addition to the sequences encoding proteins of the disclosure, further sequences such as those required for the transcription and/or translation of the sequence encoding the protein. This may include a promoter, enhancer, transcription, splicing, and/or translation initiation and/or termination sequences, selectable markers, as wells sequences protecting or directing the RNA or protein within the cell. The selection and combination of these sequences is within the knowledge of the person skilled in the art, and sequences may be selected in accordance with the cell the nucleic acid or protein is intended for.

Polynucleotides of the disclosure may be delivered to cells as an isolated nucleic acid or in a vector. The isolated nucleic acid or the vector may be delivered in lipid- or lipid-based delivery system, such as a liposome. Alternatively, the vector may comprise viral proteins, such as when the vector is a viral vector. The term “vector” as used herein refers to a construction comprised of genetic material designed to direct transformation or transductions of a targeted cell. A vector contains multiple genetic elements positionally and sequentially oriented with other necessary elements such that the nucleic acid in a nucleic acid cassette can be transcribed and when necessary translated in the transfected cells. The term vector as used herein can refer to nucleic acid, e.g., DNA derived from a plasmid, cosmid, phagemid, bacteriophage, virus, retrovirus, adenovirus, adeno-associated virus, lentivirus, or other type of virus into which one or more fragments of nucleic acid may be inserted or cloned which encode for particular proteins. The term “plasmid” as used herein refers to a construction comprised of extrachromosomal genetic material, usually of a circular duplex of DNA which can replicate independently of chromosomal DNA. The plasmid does not necessarily replicate.

Any suitable vectors are envisaged as within the scope of the instant disclosure. The polynucleotides encoding a blocker receptor and/or activator receptor (or parts thereof) can be cloned into a number of types of vectors. For example, the polynucleotides can be cloned into a vector including, but not limited to a plasmid, a cosmid, a phagemid, a phage derivative, or a viral vector.

Vectors of particular interest include expression vectors, and replication vectors. Expression vectors may be provided to cells, such as immune cells, in the form of a viral vector. Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

The vector can contain one or more unique restriction sites, and may be capable of autonomous replication in a defined host or organism such that the cloned sequence is reproduced. The vector molecule can confer some well-defined phenotype on the host organism which is either selectable or readily detected. The vector may have a linear or circular configuration. The components of a vector can contain but are not limited to a DNA molecule incorporating: (1) DNA; (2) a sequence encoding a receptor, or a part of a receptor, of the disclosure; and (3) regulatory elements for transcription, translation, RNA processing, RNA stability, and replication.

Vectors can provide expression of a protein, protein fragment, or RNA encoded by a nucleic acid sequence in cells or tissue. Expression includes the efficient transcription of an inserted gene or gene fragment. Expression products may be polypeptides, RNAs, or a combination thereof. Expression of vector products can be continuous, constitutive, tissue specific, or inducible. Vectors can include elements for replication the vector in bacteria, and selection for maintenance of plasmid in bacteria.

In some embodiments, the vector comprises the following elements linked sequentially at an appropriate distance to allow functional expression: a promoter, a 5′ mRNA untranslated sequence, a translation initiation site, a nucleic acid cassette containing the sequence of a blocker receptor and/or activator receptor (or portions thereof) to be expressed, a 3′ mRNA untranslated region, and a polyadenylation signal sequence. Optionally, the vector can contain an enhancer, typically 5′ of the promoter. As used herein the term “expression vector” refers to a DNA vector that contains all of the information necessary to produce a protein or RNA encoded by the vector in a heterologous cell.

Additional elements for gene expression, e.g., enhancers, regulate the frequency, level, and tissue specificity of transcriptional initiation. Typically, these are located in the region upstream of the start site, although enhancers can be located downstream of the start site as well, either downstream of the 3′ UTR or within an intron. The spacing between promoter elements frequently is flexible, so that promoter function is preserved when elements are inverted or moved relative to one another. Depending on the promoter, individual elements can function either cooperatively or independently to activate transcription.

Any suitable promoter may be used to drive expression of the protein, or proteins, encoded by the first and/or second vectors of the disclosure. The promoter can be constitutive, tissue specific, or inducible. In some embodiments, the first vector comprises a first promoter and the second vector comprises a second promoter. In some embodiments, the first and second promoters are the same promoter. In some embodiments, the first and second promoters are not the same promoter.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1α (EF-1α). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter.

Inducible promoters are also contemplated as within the scope of the instant disclosure. The use of an inducible promoter provides a molecular switch capable of turning on expression of a protein or RNA encoded by the polynucleotide sequence to which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

Cell-specific promoters are also contemplated as within the scope of the instant disclosure. In some embodiments, the promoter is a cell-specific promoter. In some embodiments, the promoter may be an immune cell specific promoter. In some embodiments, the promoter may be a T-cell specific promoter. Examples of T-cell specific promoters include, but are not limited to, distal Lck (dLck), and CD36 promoters.

In some embodiments, the promoter may be synthetic. In some embodiments, the promoter may be a dual or bidirectional promoter. Such promoter systems are able to direct transcription of more than one coding sequence, wherein the bidirectional promoter is positioned in the intergenic region between two adjacent genes and transcribed in opposite directions driving simultaneous transcription. Further, bidirectional promoters may often exhibit similar levels of expression patterning due to the co-expression of the adjacent genes. Examples of bidirectional promoter systems include, but are not limited to, pBI-CMV1, wherein transcription is driven by one of two constitutively active human cytomegalovirus promoters, P_{CMV IE} and P_{minCMV}, which are oriented just upstream of the two coding regions of interest. Similarly, in some embodiments, two minimal promoters may be used to drive expression of two coding sequences in opposite directions, wherein the promoters are both proximal to an intergenic enhancer element.

Additional sequence elements may be deployed to further enhance expression proteins or RNAs from the polynucleotides of the disclosure. Post-transcriptional regulation can have a profound impact on gene expression. In particular, the untranslated regions (UTR) of mRNA plays a role in efficacy of translation, stability, and localization. The 5′ UTR is a regulatory region of DNA situated at the 5′ end of all protein coding sequence that is transcribed into mRNA but not translated into protein. 5′ UTRs may contain various regulatory elements, e.g., 5′ cap structure, G-quadruplex structure (G4), stem-loop structure, and internal ribosome entry sites (IRES), which, in turn, may control translation initiation. The 3′ UTR, situated downstream of the protein coding sequence, has been found to be involved in numerous regulatory processes including transcript cleavage, stability and polyadenylation, translation, and mRNA localization. The 3′ UTR serves as a binding site for numerous regulatory proteins and small non-coding RNAs, e.g., microRNAs.

Particular features that impact post-transcriptional regulation include generalized secondary structure, open reading frames, internal ribosome entry sites, and cis-acting elements, all of which potentially impact the efficacy of expression. UTR sequences of the polynucleotides of the disclosure may, for example, be engineered to increase protein synthesis by increasing both the time that the mRNA remains in translating polysomes (message stability) and/or the rate at which ribosomes initiate translation on the message (message translation efficiency). Examples of UTR modifications may include, but are not limited to, mRNAs comprising an exogenous open reading frame (ORF) flanked by a 5′ UTR and a 3′ UTR, wherein the 5′ UTR and the 3′ UTR are derived from a naturally abundant mRNA in a tissue. The tissue may be, for example, lymphoid tissue. The lymphoid tissue may be, for example, a lymphocyte (e.g., a B-lymphocyte, a helper T-lymphocyte, a cytotoxic T-lymphocyte, a regulatory T-lymphocyte, or a natural killer cell), a macrophage, a monocyte, a dendritic cell, a neutrophil, an eosinophil or a reticulocyte. The naturally abundant mRNA may have, for example, a tissue half-life of at least about 9 hours, or from about 5 to about 60 hours.

Polynucleotides containing post-transcriptional regulatory elements (PREs) have been found to enhance expression of recombinant molecules operably linked thereto. Such PREs may comprise cis-acting post-transcriptional regulatory elements and modified variants thereof. Examples of such include, but are not limited to, PREs from hepatitis viruses, such as the woodchuck hepatitis virus (WHV), including modified variants of a woodchuck hepatitis virus post-transcriptional regulatory element (WPRE). Transcription of the WPRE sequence results in a tertiary structure, enhancing mRNA stability, thereby enhancing expression, particularly in viral vectors.

The expression of polynucleotides encoding a blocker receptor and/or an activator receptor (or parts thereof) described herein can be achieved by operably linking a nucleic acid encoding a blocker receptor and/or a activator receptor (or parts thereof) to a promoter, and incorporating the construct into an expression vector. The vectors can be suitable for replication and integration eukaryotes. Typical cloning vectors contain transcription and translation terminators, initiation sequences, and promoters useful for regulation of the expression of the desired nucleic acid sequence.

A vector as used herein can include DNA sequence elements which enable extrachromosomal (episomal) replication of the DNA in a suitable host cells, such as a bacterial or eukaryotic cell. Vectors capable of episomal replication are maintained as extrachromosomal molecules and can replicate. These vectors are not eliminated by simple degradation but continue to be copied. These elements may be derived from a viral or mammalian genome. These provide prolonged or “persistent” expression.

Further, the expression vector may be provided to a cell in the form of a viral vector. Viruses, which are useful as vectors include, but are not limited to, retroviruses, gammaretroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

A “viral vector” in this sense is one that is physically incorporated in a viral particle by the inclusion of a portion of a viral genome within the vector, e.g., a packaging signal, and is not merely DNA or a located gene taken from a portion of a viral nucleic acid. Thus, while a portion of a viral genome can be present in a vector of the present invention, that portion does not encode one or more viral proteins, such as structural proteins or proteins required for replication, and thus is unable to produce an infectious viral particle. A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In one embodiment, lentiviral vectors are used. A number of lentiviral vectors are known in the art.

In some embodiments, the vector is a “lentiviral” vector or “lentivirus,” the terms being used interchangeably. Lentiviruses are complex retroviruses, which, in addition to the common retroviral genes gag, pol, and env, contain other genes with regulatory or structural function. The higher complexity enables the virus to modulate its life cycle, as in the course of latent infection. Some examples of lentivirus include the Human Immunodeficiency Viruses: HIV-1, HIV-2 and the Simian Immunodeficiency Virus: SIV. Lentiviral vectors have been generated by multiply attenuating the HIV virulence genes, for example, the genes env, vif, vpr, vpu and nef are deleted making the vector biologically safe.

As used herein, the term “lentiviral vector” is intended to mean a nucleic acid that encodes a lentiviral cis nucleic acid sequence required for genome packaging. A lentiviral vector also can encode other cis nucleic acid sequences beneficial for gene delivery, including for example, cis sequences required for reverse transcription, proviral integration or genome transcription. A lentiviral vector performs transduction functions of a lentiviral vector. As such, the exact makeup of a vector genome will depend on the genetic material desired to be introduced into a target cell. Therefore, a vector genome can encode, for example, additional polypeptides or functions other than that required for packaging, reverse transcription, integration, or transcription. Such functions generally include coding for cis elements required for expression of a nucleic acid of interest. The lentiviral cis sequences or elements can be derived from a lentivirus genome or other virus or vector genome so long as the lentiviral vector genome can be packaged by a packaging cell line into a lentiviral particle and introduced into a target cell. IN some embodiments, the lentiviral vector is pseudotyped, i.e. bears glycoproteins (GPs) derived from other enveloped viruses which are used to target the vector to a particular cell type.

Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity. Further, they do not transfer viral genes, therefore avoiding the problem of generating transduced cells that can be destroyed by cytotoxic T-cells. In addition, lentiviruses, in contrast to other retroviruses, are capable of transducing non-dividing cells.

Lentiviral vectors are known in the art, see U.S. Pat. Nos. 6,013,516; and 5,994,136, which are incorporated herein by reference. In general, these vectors are plasmid-based or virus-based, and are configured to carry the essential sequences for incorporating foreign nucleic acid, for selection and for transfer of the nucleic acid into a host cell.

In some cases, a lentiviral vector is introduced into a cell concurrently with one or more lentiviral packaging plasmids, which may include, without limitation, pMD2.G, pRSV-rev, pMDLG-pRRE, and pRRL-GOI. Introduction of a lentiviral vector alone or in combination with lentiviral packaging plasmids into a suitable cell may cause the lentiviral vector to be packaged into a lentiviral particle.

“Adeno-associated viruses” (AAV), from the parvovirus family, are small viruses with a genome of single stranded DNA. An “AAV vector” or simply “vector” is derived from the wild type AAV by using molecular methods to remove the wild type AAV genome, and replacing with a non-native nucleic acid, such as a therapeutic gene expression cassette. Typically, the inverted terminal repeats of the wild type AAV genome are retained in the AAV vector. An AAV vector is distinguished from an AAV, since all or a part of the viral genome has been replaced with a transgene, which is a non-native nucleic acid with respect to the AAV nucleic acid sequence.

AAVs are a helper virus for replication, so natural infections take place in the context of infection with a helper virus such as adenovirus. Infection with adeno-associated virus causes no known pathologies. Adeno-associated virus (AAV) vectors are scalable, efficient, non-cytopathic gene delivery vehicles used primarily for the treatment of genetic diseases. Their ability to transduce non-dividing cells and persist episomally results in long-term transgene expression in animals. A broad spectrum of animal models of human diseases has been successfully treated by AAV vectors, including diseases of the brain, heart, lung, eye and liver.

AAVs may include, but are not limited to, AAV type 1, AAV type 2, AAV type 3 (including types 3A and 3B), AAV type 4, AAV type 5, AAV type 6, AAV type 7, AAV type 8, AAV type 9, AAV type 10, AAV type 11, avian AAV, bovine AAV, canine AAV, equine AAV, ovine AAV, Clade F AAV and any other AAV now known or later discovered.

The genomic sequences of various serotypes of AAV and the autonomous parvoviruses, as well as the sequences of the native terminal repeats (TRs), Rep proteins, and capsid subunits are known in the art. Such sequences may be found in the literature or in public databases such as GenBank® Database. See, e.g., GenBank Database Accession Numbers NC 002077, NC_001401, NC_001729, NC 001863, NC 001829, NC_001862, NC 000883, NC 001701, NC_001510, NC 006152, NC_006261, AF063497, U89790, AF043303, AF028705, AF028704, J02275, J01901, J02275, X01457, AF288061, AH009962, AY028226, AY028223, NC_001358, NC 001540, AF513851, AF513852, AY530579; the disclosures of which are incorporated by reference herein for teaching parvovirus and AAV nucleic acid and amino acid sequences. See also, international patent publications WO 00/28061, WO 99/6160 and WO 98/11244; and U.S. Pat. No. 6,156,303; the disclosures of which are incorporated by reference herein.

The capsid structures of autonomous parvoviruses and AAV are described in more detail in BERNARD N. FIELDS et al., Virology, Volume 2, Chapters 69 & 70 (4th ed., Lippincott-Raven Publishers).

The term “tropism” as used herein refers to preferential or selective entry of the virus into certain cells or tissues, optionally followed by expression (e.g., transcription and, optionally, translation) of a sequence(s) carried by the viral genome in the cell, e.g., for a recombinant virus, expression of a heterologous nucleotide sequence of interest. In some cases, viral vectors can be “pseudotyped”, i.e. express heterologous glycoproteins from a different virus, to achieve a desired tropism.

Those skilled in the art will appreciate that transcription of a heterologous nucleic acid sequence from the viral genome may not be initiated in the absence of trans-acting factors, e.g., for an inducible promoter or otherwise regulated nucleic acid sequence. In the case of a rAAV genome, gene expression from the viral genome may be from a stably integrated provirus, from a non-integrated episome, as well as any other form in which the virus may take within the cell.

A “rAAV vector genome” or “rAAV genome” is an AAV genome (i.e., vDNA) that comprises one or more heterologous nucleic acid sequences. rAAV vectors generally require only the terminal repeat(s) (TR(s)) in cis to generate virus. All other viral sequences are dispensable and may be supplied in trans. Typically, the rAAV vector genome will only retain the one or more TR sequence so as to maximize the size of the transgene that can be efficiently packaged by the vector. The structural and non-structural protein coding sequences may be provided in trans (e.g., from a vector, such as a plasmid, or by stably integrating the sequences into a packaging cell). In some embodiments of the invention, the rAAV vector genome comprises at least one TR sequence (e.g., AAV TR sequence), optionally two TRs (e.g., two AAV TRs), which typically will be at the 5′ and 3′ ends of the vector genome and flank the heterologous nucleic acid, but need not be contiguous thereto. The TRs can be the same or different from each other. AAV TR sequences need not be from a source identical to the structural and/or non-structural protein coding sequences of the virus.

The term “terminal repeat” or “TR” includes any viral terminal repeat or synthetic sequence that forms a hairpin structure and functions as an inverted terminal repeat (i.e., mediates the desired functions such as replication, virus packaging, integration and/or provirus rescue, and the like). The TR can be an AAV TR or a non-AAV TR. For example, a non-AAV TR sequence such as those of other parvoviruses (e.g., canine parvovirus (CPV), mouse parvovirus (MVM), human parvovirus B-19) or any other suitable virus sequence (e.g., the SV40 hairpin that serves as the origin of SV40 replication) can be used as a TR, which can further be modified by truncation, substitution, deletion, insertion and/or addition. Further, the TR can be partially or completely synthetic, such as the “double-D sequence” as described in U.S. Pat. No. 5,478,745.

An “AAV terminal repeat” or “AAV TR” may be from any AAV, including but not limited to serotypes 1, 2, 3, 4, 5, 6, 7, 8, 9, 10, 11, Clade F, or any other AAV now known or later discovered. An AAV terminal repeat need not have the native terminal repeat sequence (e.g., a native AAV TR sequence may be altered by insertion, deletion, truncation and/or missense mutations), as long as the terminal repeat mediates the desired functions, e.g., replication, virus packaging, integration, and/or provirus rescue, and the like.

The virus vectors of the invention can further be “targeted” virus vectors (e.g., having a directed tropism) and/or a “hybrid” parvovirus (i.e., in which the viral TRs and viral capsid are from different parvoviruses) as described in international patent publication WO 00/28004.

The virus vectors of the invention can further be duplexed parvovirus particles as described in international patent publication WO 01/92551 (the disclosure of which is incorporated herein by reference in its entirety). Thus, in some embodiments, double stranded (duplex) genomes can be packaged into the virus capsids of the invention.

Similarly, recombinant or synthetic viruses and virus-like particles (VLPs) may be used as vectors. VLPs are multiprotein structures that mimic the organization and structure of standard natural viruses (e.g., mature virion) but lack the viral genome. Therefore, VLPs are nonreplicative in nature, which make them safe for administration. Recombinant or synthetic viruses and virus-like VLPs may comprise one or more of the polypeptides, nucleic acids, or vectors of the invention. VLPs represent a highly attenuated form of a virus. Viral proteins from several viruses are known to form VLPs, including human papillomavirus, HIV, human polyomavirus, rotavirus, parvovirus, canine parvovirus, and hepatitis E virus.

Although VLPs can be naturally occurring, they may also be synthesized through the expression of viral structural proteins which are capable of self-assembling into virus like structures. Examples may include transfecting cells with GAG ProPol and a viral envelope protein with a construct encoding a guide RNA, wherein GAG and the viral envelop localize at the membrane where the assembly of a virus-derived particle takes place.

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York). One method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Introduction of a viral vector into a eukaryotic host cell can be referred to as “transduction.”

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In order to assess the expression of introduced polypeptides, the expression vector to be introduced into a cell can also contain either a selectable marker gene or a reporter gene, or both, to facilitate identification and selection of expressing cells from the population of cells sought to be transfected or infected through viral vectors. Both selectable markers and reporter genes may be flanked with appropriate regulatory sequences to enable expression in the host cells. Useful selectable markers include, for example, antibiotic-resistance genes, such as neo and the like.

Regardless of the method used to introduce exogenous nucleic acids into a host cell, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or other assays described herein to identify agents falling within the scope of the invention.

### Additional Components of the Dual Vector System

One advantage of the dual vector system of the disclosure is that it provides more space to include additional sequences, as opposed to a single vector. Accordingly, the disclosure provides additional sequence elements for inclusion in the dual vector system of the instant disclosure. Additional components include, but are not limited to, additional genes, markers, reporters, enzymes, cytokines, short interfering RNAs (shRNAs) and the like, as well as inducible kill switch genes that can induce apoptosis in the cell expressing the vector. In some embodiments, the first and/or second vector comprises a sequence encoding an additional gene. In some embodiments, the additional gene comprises a marker, a reporter, a short interfering RNA, or an inducible kill switch.

In order to assess the expression or function of a blocker and/or activator receptor of the dual vector system of the instant disclosure, the first and/or second vector can also contain either a selectable marker gene or a reporter gene, or both. Markers and reporters facilitate identification and selection of expressing cells from the population of cells transfected or transduced cells. Both selectable markers and reporter genes may include appropriate regulatory sequences to enable expression in the host cells. Useful selectable markers include, for example, antibiotic-resistance genes, such as neo and the like.

Reporter genes can be used for identifying potentially transformed cells and for evaluating the functionality of regulatory sequences. Furthermore, the use of reporter genes in the context of the dual vector system of the instant disclosure may be used to identify successful transformation (i.e., transfection or transduction) of the cell both vectors. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or fluorescent proteins such as green fluorescent protein (GFP), mCherry, mTagBFP2, mTurquoise2, mCerulean3, EGFP, mWasabi, Superfolder GFP, mNeonGreen, mClover3, Venus, Citrine, mKOk, tdTomato, TagRFP-T, mRuby3, mScarlet, FusionRed, mCherry, mStable, mKate2, mMaroon1, mCardinal, T-Sapphire, mCyRFPT, LSSmOrange, mBeRFP, luciferase, and the like. Suitable expression systems are well known and may be prepared using known techniques or obtained commercially. Suitable promoter regions may be linked to a reporter gene and used to evaluate agents for the ability to modulate promoter-driven transcription. “Reporter” genes are typically proximally linked a regulatory sequence related to a biological process to be assayed, whereby the reporter gene is deployed as a tool for enabling qualitative and/or quantitative measurement of the biological process by virtue of the reporter's ability to be measured in some capacity. Cells exhibiting a fluorescent reporter gene are distinguishable from other cells by fluorescence emission when irradiated with wavelengths of light that excite the fluorescent protein, and can be identified by methods known in the art, such as fluorescence activated cell sorting (FACS). Similarly, cells exhibiting a luminescent reporter gene are distinguishable from other cells by measuring photon emission produced in the presence of a substrate (e.g., luciferin for luciferase). The substrate may be exogenously added or co-expressed in a dual-vector system to produce an auto-luminescent response.

In some embodiments, the dual vector system of the instant disclosure comprises a sequence encoding a fluorescent or luminescent reporter protein. In some embodiments, the fluorescent reporter protein is EGFP. In some embodiments, the luminescent reporter protein is luciferase. In some embodiments, the sequence encoding the reporter is located on a vector comprising a gene encoding a blocker receptor and a portion of an activator receptor. In some embodiments, the sequence encoding the reporter is located on a vector comprising a gene encoding a portion of an activator, wherein the vector does not include a encoding a blocker receptor. In some embodiments, the sequence encoding the reporter is located on a first vector comprising a gene encoding a blocker and a portion of an activator; and a second vector comprising a gene encoding a portion of an activator, wherein the vector is absent a gene encoding a blocker; and wherein the exogeneous reporters of the first and second vector are different and distinguishable by different fluorescence emission spectra.

In some embodiments, the dual vector system of the instant disclosure comprises a sequence encoding a luminescent reporter protein. In some embodiments, the dual vector system further comprises an exogenous gene encoding a cognate substrate of the luminescent reporter protein. In some embodiments, the luminescent reporter protein is a luciferase. In some embodiments, cognate substrate is a luciferin. In some embodiments, the sequence encoding the luminescent reporter protein is located on a vector comprising a gene encoding a blocker and a portion of an activator. In some embodiments, the sequence encoding the luminescent reporter protein is located on a vector comprising a gene encoding a portion of an activator, wherein the vector is absent a gene encoding a blocker.

An additional way to identify and select cells that express transfected DNA is to provide an exogenous gene encoding a selectable marker on the vector used for transfection or on a separate vector. In some embodiments, a selectable marker may be present on one or both of the vectors of the dual vector system of the instant disclosure. In some embodiments, wherein both of the vectors of the dual vector system comprise a gene encoding a selectable marker, the dual vectors comprise different selectable markers. Various selectable markers confer resistance to cells exposed to an appropriate selective pressure. Examples of selectable markers include, but are not limited to, genes that confer resistance to selection drugs, or genes that compensate for an essential function that is defective in the cell (e.g., amino acid synthesis). Selection antibiotics for eukaryotic cells may include, but are not limited to hygromycin B, puromycin, blasticidin, phleomycin D1, and G418 sulfate.

In some embodiments, the additional sequence element of the dual vector system comprises one or more genes encoding a selectable marker. In some embodiments, the additional sequence element of the dual vector system comprises one or more selectable markers. In some embodiments, the sequence encoding the selectable marker gene is located on a vector comprising a sequence encoding a blocker receptor and a portion of an activator receptor. In some embodiments, the sequence encoding the selectable marker gene is located on a vector comprising a sequence encoding a portion of an activator receptor, wherein the vector is absent a sequence encoding a blocker receptor.

Other useful molecular tools are envisaged in the instant disclosure. Such molecular tools may include systems to manipulate other genes of interest in a desired cell. In some embodiments, the additional sequence element of the dual vector system comprises a sequence or sequences encoding one or more short hairpin RNAs (shRNA), microRNAs, long non-coding RNAs, or the like. Sequences encoding shRNAs produce an artificial RNA molecule comprising a hairpin turn that can be utilized to silence target gene expression through RNA interference. Examples of shRNA may include simple stem-loop or microRNA (miRNA)-adapted shRNA. shRNA molecules are processed within a cell to form small interfering RNA which can knock down a targeted gene's expression. RNA-induced gene silencing in mammalian cells is presently believed to implicate a minimum of three different levels of control: (i) transcription inactivation (siRNA-guided DNA and histone methylation); (ii) small interfering RNA (siRNA)-induced mRNA degradation; and (iii) mRNA-induced transcriptional attenuation. RNAi is a molecular process by which an antisense RNA molecule binds to a complementary mRNA, or target, inducing mRNA degradation. Traditionally, double stranded RNA (dsRNA) molecules comprising a hairpin or complementary RNAs are recognized by DICER, an endo-ribonuclease, wherein the dsRNA molecule is cleaved to form siRNA, which is utilized by the RNA-Induced Silencing Complex (RISC). RNAi is mediated by RISC, wherein various proteins reduce the siRNA into a single stranded siRNA molecule which is in turn used to locate and bind with a complementary mRNA sequence. The siRNA-RISC complex binds to its specific target inducing cleavage of the target mRNA via Argonaute proteins. The resulting cleavage of the target mRNA destroys the transcript preventing it from being translated into a protein.

In some embodiments, the dual vector system of the instant disclosure comprises exogenous sequences encoding one or more shRNAs. In some embodiments, the one or more shRNAs are specific to an mRNA transcribed from the genes encoding beta-2-microglobulin (B2M), HLA-A, HLA-B, or HLA-C. In some embodiments, the one or more shRNAs are specific to an mRNA transcribed from the gene encoding B2M. In some embodiments, the one or more shRNAs are specific to an mRNA transcribed from the gene encoding HLA-A. In some embodiments, the one or more shRNAs are specific to an mRNA transcribed from the gene encoding HLA-B. In some embodiments, the one or more shRNAs are specific to an mRNA transcribed from the gene encoding HLA-C. In some embodiments, the sequence encoding the one or more shRNAs is located on a vector comprising a sequence encoding a blocker receptor and a portion of an activator receptor. In some embodiments, the sequence encoding the one or more shRNAs is located on a vector comprising a sequence encoding a portion of an activator receptor, wherein the vector is absent a sequence encoding a blocker receptor. In some embodiments, sequence encoding the one or more shRNAs is located on a first vector comprising a sequence encoding a blocker receptor and a portion of an activator receptor; and a second vector comprising a sequence encoding a portion of an activator receptor, wherein the vector is absent a sequence encoding a blocker receptor.

In some embodiments, the first or second vector comprises a sequence encoding a gene that can inducibly cause apoptosis of a cell expressing the gene (a “kill switch”). In some embodiments, the kill switch comprises a truncated BH3-interacting domain death agonist (tBID), to a steroid hormone receptor domain, such as the estrogen receptor (ESR1) ligand binding domain. Using a ligand binding from a nuclear hormone receptor such as ESR1 brings the activity of tBID under control of a ligand such as tamoxifen.

An exemplary kill switch gene comprising a tBID domain and an ESR1 ligand binding domain comprises a sequence of

MSQEDIIRNIARHLAQVGDSMDRSIPPGEKTMLVLALLLAKKVASHTPSL
LRDVFHTTVNFINQNLGSKKNSLALSLTADQMVSALLDAEPPILYSEYDP
TRPFSEASMMGLLTNLADRELVHMINWAKRVPGFVDLTLHDQVHLLECAW
LEILMIGLVWRSMEHPGKLLFAPNLLLDRNQGKCVEGMVEIFDMLLATSS
RERMMNLQGEEFVCLKSIILLNSGVYTFLSSTLKSLEEKDHIHRVLDKIT
DTLIHLMAKAGLTLQQQHQRLAQLLLILSHIRHMSNKGMEHLYSMKCKNV
VPLYDLLLEMLDAHRLHAPT.

### Cells

Provided herein are cells comprising the first and second vectors of the dual vector system described herein. In some embodiments, the first vector comprises a polynucleotide comprising a sequence encoding an inhibitory receptor and a sequence encoding a first part of an activator receptor, and the second vector comprises a polynucleotide comprising a sequence encoding a second part of the activator receptor. When the cells are transformed with both vectors of the system, the first and second parts of the activator receptor together form a functional activator receptor that is expressed by the cell. In some embodiments, the cells express an activator receptor and an inhibitory receptor. In contrast, cells that are transformed with only one of the two vectors do not express a complete activator receptor, although, depending on the vector, the cells may express an inhibitory receptor and/or inactive activator receptor fragment.

The disclosure provides cells expressing the activator and inhibitory receptors described herein. In some embodiments, the activator receptor is a CAR. In some embodiments, the activator receptor is a TCR. In some embodiments, the inhibitory receptor comprises a LIR1 intracellular domain, for example an scFv domain fused to LIR1 hinge, transmembrane and intracellular domain. In some embodiments, the cells express an additional protein or RNA encoded by the vector system, for example an siRNA, marker, reporter or kill switch protein.

In some embodiments, the cells are eukaryotic cells. In some embodiments, the eukaryotic cells are mammalian cells. In some embodiments, the cells are human cells. In some embodiments, the cells are immune cells.

As used herein, the term “immune cell” refers to a cell involved in the innate or adaptive (acquired) immune systems. Exemplary innate immune cells include phagocytic cells such as neutrophils, monocytes and macrophages, Natural Killer (NK) cells, polymophonuclear leukocytes such as neutrophils eosinophils and basophils and mononuclear cells such as monocytes, macrophages and mast cells. Immune cells with roles in acquired immunity include lymphocytes such as T-cells and B-cells.

As used herein, a “T-cell” refers to a type of lymphocyte that originates from a bone marrow precursor that develops in the thymus gland. There are several distinct types of T-cells which develop upon migration to the thymus, which include, helper CD4+ T-cells, cytotoxic CD8+ T cells, memory T cells, regulatory CD4+ T-cells and stem memory T-cells. Different types of T-cells can be distinguished by the ordinarily skilled artisan based on their expression of markers. Methods of distinguishing between T-cell types will be readily apparent to the ordinarily skilled artisan.

Methods transforming populations of immune cells, such as T cells, with the vectors of the instant disclosure will be readily apparent to the person of ordinary skill in the art. For example, CD3+ T cells can be isolated from PBMCs using a CD3+ T cell negative isolation kit (Miltenyi), according to manufacturer's instructions. T cells can be cultured at a density of 1×10\hat{ }6 cells/mL in X-Vivo 15 media supplemented with 5% human A/B serum and 1% Pen/strep in the presence of CD3/28 Dynabeads (1:1 cell to bead ratio) and 300 Units/mL of IL-2 (Miltenyi). After 2 days, T cells can be transduced with viral vectors, such as lentiviral vectors using methods known in the art. In some embodiments, the viral vector is transduced at a multiplicity of infection (MOI) of 5. Cells can then be cultured in IL-2 or other cytokines such as combinations of IL-7/15/21 for an additional 5 days prior to enrichment. Methods of isolating and culturing other populations of immune cells, such as B cells, or other populations of T cells, will be readily apparent to the person of ordinary skill in the art. Although this method outlines a potential approach it should be noted that these methodologies are rapidly evolving. For example excellent viral transduction of peripheral blood mononuclear cells can be achieved after 5 days of growth to generate a >99% CD3+ highly transduced cell population.

Methods of activating and culturing populations of T cells comprising the TCRs, CARs, fusion proteins or vectors encoding same, will be readily apparent to the person of ordinary skill in the art.

Whether prior to or after genetic modification of T cells, the T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041, 10,040,846; and U.S. Pat. Appl. Pub. No. 2006/0121005.

In some embodiments, T cells of the instant disclosure are expanded and activated in vitro. Generally, the T cells of the instant disclosure are expanded in vitro by contact with a surface having attached thereto an agent that stimulates a CD3/TCR complex associated signal and a ligand that stimulates a co-stimulatory molecule on the surface of the T cells. In particular, T cell populations may be stimulated as described herein, such as by contact with an anti-CD3 antibody. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of either CD4+ T cells or CD8+ T cells, an anti-CD3 antibody and an anti-CD28 antibody can be used. Examples of an anti-CD28 antibody include 9.3, B-T3, XR-CD28 (Diaclone, Besangon, France) can be used as can other methods commonly known in the art (Berg et al., Transplant Proc. 30(8):3975-3977, 1998; Haanen et al., J. Exp. Med. 190(9):13191328, 1999; Garland et al., J. Immunol Meth. 227(1-2):53-63, 1999).

In some embodiments, the primary stimulatory signal and the co-stimulatory signal for the T cell may be provided by different protocols. For example, the agents providing each signal may be in solution or coupled to a surface. When coupled to a surface, the agents may be coupled to the same surface (i.e., in “cis” formation) or to separate surfaces (i.e., in “trans” formation). Alternatively, one agent may be coupled to a surface and the other agent in solution. In some embodiments, the agent providing the co-stimulatory signal is bound to a cell surface and the agent providing the primary activation signal is in solution or coupled to a surface. In certain embodiments, both agents can be in solution. In another embodiment, the agents may be in soluble form, and then cross-linked to a surface, such as a cell expressing Fc receptors or an antibody or other binding agent which will bind to the agents. In this regard, see for example, U.S. Patent Application Publication Nos. 20040101519 and 20060034810 for artificial antigen presenting cells (aAPCs) that are contemplated for use in activating and expanding T cells in the present invention.

In some embodiments, the two agents are immobilized on beads, either on the same bead, i.e., “cis,” or to separate beads, i.e., “trans.” By way of example, the agent providing the primary activation signal is an anti-CD3 antibody or an antigen-binding fragment thereof and the agent providing the co-stimulatory signal is an anti-CD28 antibody or antigen-binding fragment thereof; and both agents are co-immobilized to the same bead in equivalent molecular amounts. In one embodiment, a 1:1 ratio of each antibody bound to the beads for CD4+ T cell expansion and T cell growth is used. In some embodiments, the ratio of CD3:CD28 antibody bound to the beads ranges from 100:1 to 1:100 and all integer values there between. In one aspect of the present invention, more anti-CD28 antibody is bound to the particles than anti-CD3 antibody, i.e., the ratio of CD3:CD28 is less than one. In certain embodiments of the invention, the ratio of anti CD28 antibody to anti CD3 antibody bound to the beads is greater than 2:1.

Ratios of particles to cells from 1:500 to 500:1 and any integer values in between may be used to stimulate T cells or other target cells. As those of ordinary skill in the art can readily appreciate, the ratio of particles to cells may depend on particle size relative to the target cell. For example, small sized beads could only bind a few cells, while larger beads could bind many. In certain embodiments the ratio of cells to particles ranges from 1:100 to 100:1 and any integer values in-between and in further embodiments the ratio comprises 1:9 to 9:1 and any integer values in between, can also be used to stimulate T cells. In some embodiments, a ratio of 1:1 cells to beads is used. One of skill in the art will appreciate that a variety of other ratios may be suitable for use in the present invention. In particular, ratios will vary depending on particle size and on cell size and type.

In further embodiments of the present invention, the cells, such as T cells, are combined with agent-coated beads, the beads and the cells are subsequently separated, and then the cells are cultured. In an alternative embodiment, prior to culture, the agent-coated beads and cells are not separated but are cultured together. In a further embodiment, the beads and cells are first concentrated by application of a force, such as a magnetic force, resulting in increased ligation of cell surface markers, thereby inducing cell stimulation.

By way of example, cell surface proteins may be ligated by allowing paramagnetic beads to which anti-CD3 and anti-CD28 are attached to contact the T cells. In one embodiment the cells (for example, CD4+ T cells) and beads (for example, DYNABEADS CD3/CD28 T paramagnetic beads at a ratio of 1:1) are combined in a buffer. Again, those of ordinary skill in the art can readily appreciate any cell concentration may be used. In certain embodiments, it may be desirable to significantly decrease the volume in which particles and cells are mixed together (i.e., increase the concentration of cells), to ensure maximum contact of cells and particles. For example, in one embodiment, a concentration of about 2 billion cells/ml is used. In another embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. In some embodiments, cells that are cultured at a density of 1×10⁶ cells/mL are used.

In some embodiments, the mixture may be cultured for several hours (about 3 hours) to about 14 days or any hourly integer value in between. In another embodiment, the beads and T cells are cultured together for 2-3 days. Conditions appropriate for T cell culture include an appropriate media (e.g., Minimal Essential Media or RPMI Media 1640 or, X-vivo 15, (Lonza)) that may contain factors necessary for proliferation and viability, including serum (e.g., fetal bovine or human serum), interleukin-2 (IL-2), insulin, IFN-γ, IL-4, IL-7, GM-CSF, IL-10, IL-12, IL-15, TGFβ, and TNF-α or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, α-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. In some embodiments, the media comprises X-VIVO-15 media supplemented with 5% human A/B serum, 1% penicillin/streptomycin (pen/strep) and 300 Units/ml of IL-2 (Miltenyi).

The T cells are maintained under conditions necessary to support growth, for example, an appropriate temperature (e.g., 37° C.) and atmosphere (e.g., air plus 5% CO2).

In some embodiments, the T cells are autologous. In some embodiments, the T cells are allogeneic. Prior to expansion and genetic modification, a source of T cells is obtained from a subject. Immune cells such as T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In certain embodiments of the present invention, any number of T cell lines available in the art, may be used. In certain embodiments of the present invention, T cells can be obtained from a unit of blood collected from a subject using any number of techniques known to the skilled artisan, such as Ficoll™ separation.

In some embodiments, cells from the circulating blood of an individual are obtained by apheresis. The apheresis product typically contains lymphocytes, including T cells, monocytes, granulocytes, B cells, other nucleated white blood cells, red blood cells, and platelets. In some embodiments, the cells collected by apheresis may be washed to remove the plasma fraction and to place the cells in an appropriate buffer or media for subsequent processing steps. In some embodiments, the cells are washed with phosphate buffered saline (PBS). In alternative embodiments, the wash solution lacks calcium and may lack magnesium or may lack many if not all divalent cations. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca2+-free, Mg2+-free PBS, PlasmaLyte A, or other saline solution with or without buffer. Alternatively, the undesirable components of the apheresis sample may be removed and the cells directly resuspended in culture media.

In some embodiments, immune cells such as T cells are isolated from peripheral blood lymphocytes by lysing the red blood cells and depleting the monocytes, for example, by centrifugation through a PERCOLL™ gradient or by counterflow centrifugal elutriation. Specific subpopulations of immune cells, such as T cells, B cells, or CD4+ T cells can be further isolated by positive or negative selection techniques. For example, in one embodiment, T cells are isolated by incubation with anti-CD4-conjugated beads, for a time period sufficient for positive selection of the desired T cells.

Enrichment of an immune cell population, such as a T cell population, by negative selection can be accomplished with a combination of antibodies directed to surface markers unique to the negatively selected cells. One method is cell sorting and/or selection via negative magnetic immune-adherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD 14, CD20, CD 11b, CD 16, HLA-DR, and CD8.

For isolation of a desired population of immune cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads.

In some embodiments, the cells may be incubated on a rotator for varying lengths of time at varying speeds at either 2-10° C. or at room temperature.

T cells for stimulation, or PBMCs from which immune cells such as T cells are isolated, can also be frozen after a washing step. Wishing not to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% Dextran 40 and 5% Dextrose, 20% Human Serum Albumin and 7.5% DMSO, or 31.25% Plasmalyte-A, 31.25% Dextrose 5%, 0.45% NaCl, 10% Dextran 40 and 5% Dextrose, 20% Human Serum Albumin, and 7.5% DMSO or other suitable cell freezing media containing for example, Hespan and PlasmaLyte A, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

### Immunotherapy

Provided herein are methods of treating a subject in need thereof, comprising administering to the subject a therapeutically effective amount of a composition comprising immune cells that have been transformed with the vector system of the disclosure.

Provided herein are methods of making an immune cell therapy to treat a subject in need thereof. In some embodiments, the method comprises transforming immune cells with the vector system described herein.

The current method for adoptive cell therapy using autologous cells includes isolating immune cells from patient blood, performing a series of modifications on the isolated cells, and administering the cells to a patient (Papathanasiou et al. Cancer Gene Therapy. 27:799-809 (2020)). Providing immune cells from a subject suffering from or at risk for cancer or a hematological malignancy requires isolation of immune cell from the patient's blood, and can be accomplished through methods known in the art, for example, by leukapheresis. During leukapheresis, blood from a subject is extracted and the peripheral blood mononuclear cells (PBMCs) are separated, and the remainder of the blood is returned to the subject's circulation. The PBMCs are stored either frozen or cryopreserved as a sample of immune cells and provided for further processing steps, such as, e.g. the modifications described herein.

In some embodiments, the method of treating a subject described herein comprises modifications to immune cells from the subject comprising a series of modifications comprising enrichment, activation, genetic modification, expansion, formulation, and cryopreservation.

The disclosure provides enrichment steps that can be, for example, washing and fractionating methods known in the art for preparation of subject PBMCs for downstream procedures, e.g. the modifications described herein. For example, without limitation, methods can include devices to remove gross red blood cells and platelet contaminants, systems for size-based cell fractionation for the depletion of monocytes and the isolation of lymphocytes, and/or systems that allow the enrichment of specific subsets of T cells, such as, e.g. CD4+, CD8+, CD25+, or CD62L+ T cells. Following the enrichment steps, a target sub-population of immune cells will be isolated from the subject PMBCs for further processing. Those skilled in the art will appreciate that enrichment steps, as provided herein, may also encompass any newly discovered method, device, reagent or combination thereof.

The disclosure provides activation steps that can be any method known in the art to induce activation of immune cells, e.g. T cells, required for their ex vivo expansion. Immune cell activation can be achieved, for example, by culturing the subject immune cells in the presence of dendritic cells, culturing the subject immune cells in the presence of artificial antigen-presenting cells (AAPCs), or culturing the immune cells in the presence of irradiated K562-derived AAPCs. Other methods for activating subject immune cells can be, for example, culturing the immune cells in the presence of isolated activating factors and compositions, e.g. beads, surfaces, or particles functionalized with activating factors. Activating factors can include, for example, antibodies, e.g. anti-CD3 and/or anti-CD28 antibodies. Activating factors can also be, for example, cytokines, e.g. interleukin (IL)-2 or IL-21. Activating factors can also be costimulatory molecules, such as, for example, CD40, CD40L, CD70, CD80, CD83, CD86, CD137L, ICOSL, GITRL, and CD134L. Those skilled in the art will appreciate that activating factors, as provided herein, may also encompass any newly discovered activating factor, reagent, composition, or combination thereof that can activate immune cells.

The disclosure provides genetic modification steps for modifying the subject immune cells. In some embodiments, the genetic modification comprises transducing the immune cell the vector system described herein. In some embodiments, the method comprises transducing the immune cell with a first vector comprising a sequence encoding the inhibitory receptor and a first part of the activator receptor, and a second vector comprising the second part of the activator receptor, thereby producing an immune cell expressing the activator and inhibitory receptors.

The disclosure provides expansion steps for the genetically modified subject immune cells. Genetically modified subject immune cells can be expanded in any immune cell expansion system known in the art to generate therapeutic doses of immune cells for administration. For example, bioreactor bags for use in a system comprising controller pumps, and probes that allow for automatic feeding and waste removal can be used for immune cell expansion. Cell culture flasks with gas-permeable membranes at the base may be used for immune cell expansion. Any such system known in the art that enables expansion of immune cells for clinical use is encompassed by the expansion step provided herein. Immune cells are expanded in culture systems in media formulated specifically for expansion. Expansion can also be facilitated by culturing the immune cell of the disclosure in the presence of activation factors as described herein. Those skilled in the art will appreciate that expansion steps, as provided herein, may also encompass any newly discovered culture systems, media, or activating factors that can be used to expand immune cells.

The disclosure provides formulation and cryopreservation steps for the expanded genetically modified subject immune cells. Formulation steps provided include, for example, washing away excess components used in the preparation and expansion of immune cells of the methods of treatment described herein. Any pharmaceutically acceptable formulation medium or wash buffer compatible with immune cell known in the art may be used to wash, dilute/concentration immune cells, and prepare doses for administration. Formulation medium can be acceptable for administration of the immune cells, such as, for example crystalloid solutions for intravenous infusion. Cryopreservation can optionally be used to store immune cells long-term. Cryopreservation can be achieved using known methods in the art, including for example, storing cells in a cryopreservation medium containing cryopreservation components. Cryopreservation components can include, for example, dimethyl sulfoxide or glycerol. Immune cells stored in cryopreservation medium can be cryopreserved by reducing the storage temperature to −80° C. to −180° C.

In some embodiments, the method comprises administering immune cells described herein. In some embodiments, the method comprises administering a conditioning regimen prior to administering the immune cells described herein. In some embodiments, the conditioning regimen is lymphodepletion. A lymphodepletion regimen can include, for example, administration of alemtuzumab, cyclophosphamide, benduamustin, rituximab, pentostatin, and/or fludarabine. Lymphodepletion regimen can be administered in one or more cycles until the desired outcome of reduced circulating immune cells.

In some embodiments, the conditioning regimen comprises administering an agent that specifically targets, and reduces or eliminates CD52+ cells in the subject, and the immune cells are modified to reduce or eliminate CD52 expression.

In some embodiments, the subject in need thereof has cancer. Cancer is a disease in which abnormal cells divide without control and spread to nearby tissue. In some embodiments, the cancer comprises a liquid tumor or a solid tumor. Exemplary liquid tumors include leukemias and lymphomas. Exemplary solid tumors include sarcomas and carcinomas. Cancers can arise in virtually an organ in the body, including blood, bone marrow, lung, breast, colon, bone, central nervous system, pancreas, prostate and ovary. Further cancers that are solid tumors include, for example, prostate cancer, testicular cancer, breast cancer, brain cancer, pancreatic cancer, colon cancer, thyroid cancer, stomach cancer, lung cancer, ovarian cancer, Kaposi's sarcoma, skin cancer, squamous cell skin cancer, renal cancer, head and neck cancers, throat cancer, squamous carcinomas that form on the moist mucosal linings of the nose, mouth, throat, bladder cancer, osteosarcoma, cervical cancer, endometrial cancer, esophageal cancer, liver cancer, and kidney cancer. In some embodiments, the condition treated by the methods described herein is metastasis of melanoma cells, prostate cancer cells, testicular cancer cells, breast cancer cells, brain cancer cells, pancreatic cancer cells, colon cancer cells, thyroid cancer cells, stomach cancer cells, lung cancer cells, ovarian cancer cells, Kaposi's sarcoma cells, skin cancer cells, renal cancer cells, head or neck cancer cells, throat cancer cells, squamous carcinoma cells, bladder cancer cells, osteosarcoma cells, cervical cancer cells, endometrial cancer cells, esophageal cancer cells, liver cancer cells, or kidney cancer cells.

Any cancer wherein a plurality of the cancer cells express the first, activator antigen and do not express the second, inhibitor antigen is envisaged as within the scope of the instant disclosure.

Treating cancer can result in a reduction in size of a tumor. A reduction in size of a tumor may also be referred to as “tumor regression”. Preferably, after treatment, tumor size is reduced by 5% or greater relative to its size prior to treatment; more preferably, tumor size is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Size of a tumor may be measured by any reproducible means of measurement. The size of a tumor may be measured as a diameter of the tumor.

Treating cancer can result in a reduction in tumor volume. Preferably, after treatment, tumor volume is reduced by 5% or greater relative to its size prior to treatment; more preferably, tumor volume is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Tumor volume may be measured by any reproducible means of measurement.

Treating cancer results in a decrease in number of tumors. Preferably, after treatment, tumor number is reduced by 5% or greater relative to number prior to treatment; more preferably, tumor number is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. Number of tumors may be measured by any reproducible means of measurement. The number of tumors may be measured by counting tumors visible to the naked eye or at a specified magnification. Preferably, the specified magnification is 2×, 3×, 4×, 5×, 10×, or 50×.

Treating cancer can result in a decrease in number of metastatic lesions in other tissues or organs distant from the primary tumor site. Preferably, after treatment, the number of metastatic lesions is reduced by 5% or greater relative to number prior to treatment; more preferably, the number of metastatic lesions is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. The number of metastatic lesions may be measured by any reproducible means of measurement. The number of metastatic lesions may be measured by counting metastatic lesions visible to the naked eye or at a specified magnification. Preferably, the specified magnification is 2×, 3×, 4×, 5×, 10×, or 50×.

Treating cancer can result in an increase in average survival time of a population of treated subjects in comparison to a population receiving carrier alone. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer can result in an increase in average survival time of a population of treated subjects in comparison to a population of untreated subjects. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer can result in increase in average survival time of a population of treated subjects in comparison to a population receiving monotherapy with a drug that is not a compound of the present invention, or a pharmaceutically acceptable salt, prodrug, metabolite, analog or derivative thereof. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer can result in a decrease in the mortality rate of a population of treated subjects in comparison to a population receiving carrier alone. Treating cancer can result in a decrease in the mortality rate of a population of treated subjects in comparison to an untreated population. Treating cancer can result in a decrease in the mortality rate of a population of treated subjects in comparison to a population receiving monotherapy with a drug that is not a compound of the present invention, or a pharmaceutically acceptable salt, prodrug, metabolite, analog or derivative thereof. Preferably, the mortality rate is decreased by more than 2%; more preferably, by more than 5%; more preferably, by more than 10%; and most preferably, by more than 25%. A decrease in the mortality rate of a population of treated subjects may be measured by any reproducible means. A decrease in the mortality rate of a population may be measured, for example, by calculating for a population the average number of disease-related deaths per unit time following initiation of treatment with an active compound. A decrease in the mortality rate of a population may also be measured, for example, by calculating for a population the average number of disease-related deaths per unit time following completion of a first round of treatment with an active compound.

Treating cancer can result in a decrease in tumor growth rate. Preferably, after treatment, tumor growth rate is reduced by at least 5% relative to number prior to treatment; more preferably, tumor growth rate is reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. Tumor growth rate may be measured by any reproducible means of measurement. Tumor growth rate can be measured according to a change in tumor diameter per unit time.

Treating cancer can result in a decrease in tumor regrowth. Preferably, after treatment, tumor regrowth is less than 5%; more preferably, tumor regrowth is less than 10%; more preferably, less than 20%; more preferably, less than 30%; more preferably, less than 40%; more preferably, less than 50%; even more preferably, less than 50%; and most preferably, less than 75%. Tumor regrowth may be measured by any reproducible means of measurement. Tumor regrowth is measured, for example, by measuring an increase in the diameter of a tumor after a prior tumor shrinkage that followed treatment. A decrease in tumor regrowth is indicated by failure of tumors to reoccur after treatment has stopped.

Treating or preventing a cell proliferative disorder can result in a reduction in the rate of cellular proliferation. Preferably, after treatment, the rate of cellular proliferation is reduced by at least 5%; more preferably, by at least 10%; more preferably, by at least 20%; more preferably, by at least 30%; more preferably, by at least 40%; more preferably, by at least 50%; even more preferably, by at least 50%; and most preferably, by at least 75%. The rate of cellular proliferation may be measured by any reproducible means of measurement. The rate of cellular proliferation is measured, for example, by measuring the number of dividing cells in a tissue sample per unit time.

Treating or preventing a cell proliferative disorder can result in a reduction in the proportion of proliferating cells. Preferably, after treatment, the proportion of proliferating cells is reduced by at least 5%; more preferably, by at least 10%; more preferably, by at least 20%; more preferably, by at least 30%; more preferably, by at least 40%; more preferably, by at least 50%; even more preferably, by at least 50%; and most preferably, by at least 75%. The proportion of proliferating cells may be measured by any reproducible means of measurement. Preferably, the proportion of proliferating cells is measured, for example, by quantifying the number of dividing cells relative to the number of nondividing cells in a tissue sample. The proportion of proliferating cells can be equivalent to the mitotic index.

Treating or preventing a cell proliferative disorder can result in a decrease in size of an area or zone of cellular proliferation. Preferably, after treatment, size of an area or zone of cellular proliferation is reduced by at least 5% relative to its size prior to treatment; more preferably, reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. Size of an area or zone of cellular proliferation may be measured by any reproducible means of measurement. The size of an area or zone of cellular proliferation may be measured as a diameter or width of an area or zone of cellular proliferation.

Treating or preventing a cell proliferative disorder can result in a decrease in the number or proportion of cells having an abnormal appearance or morphology. Preferably, after treatment, the number of cells having an abnormal morphology is reduced by at least 5% relative to its size prior to treatment; more preferably, reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. An abnormal cellular appearance or morphology may be measured by any reproducible means of measurement. An abnormal cellular morphology can be measured by microscopy, e.g., using an inverted tissue culture microscope. An abnormal cellular morphology can take the form of nuclear pleiomorphism.

### Kits

The disclosure provides kits and articles of manufacture comprising the dual vectors described herein, and immune cells comprising the dual vectors and proteins encoded by same, as described herein. In some embodiments, the kit comprises articles such as vials, syringes and instructions for use.

In some embodiments, the kit comprises a plurality of immune cells comprising an engineered receptor as described herein. In some embodiments, the plurality of immune cells comprises a plurality of T cells.

### Examples

### Example 1: Splitting the Activator Receptor at the Antigen Binding Domain

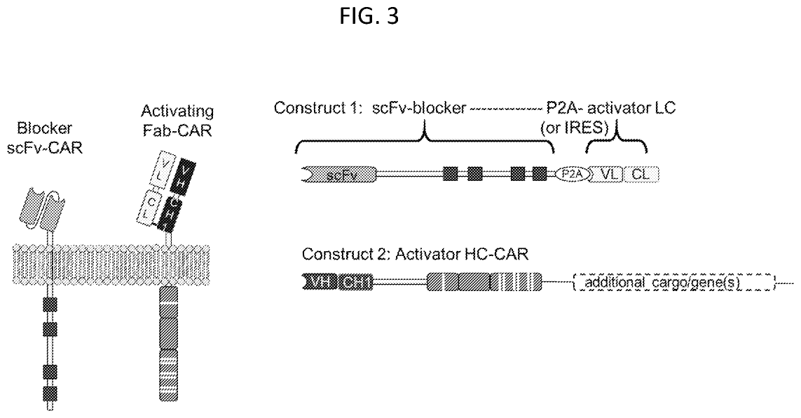

The activator receptor was split via the heavy and light chains of a Fab antigen binding domain. The blocker receptor and the light chain of the activator, separated by a P2A self-cleaving polypeptide, were included in one vector, while the heavy chain, hinge, transmembrane domain, and intracellular domain of the activator were encoded by a second vectors. A schematic of these two vectors is shown in FIG. 3. By splitting the activator Fab-CAR between the two vectors, this arrangement of elements ensures that only cells that express a blocker can also express a functional activator. It also eliminates the need to enrich for cells expressing the blocker receptor post co-transfection or co-transduction.

Jurkat cells encoding an NFAT Luciferase reporter were obtained from BPS Bioscience. In culture, Jurkat cells were maintained in RPMI media supplemented with 10% FBS, 1% Pen/Strep and 0.4 mg/mL G418/Geneticin. The ONE-Step Luciferase Assay System (BPS Bioscience) was used to evaluate Jurkat luminescence.

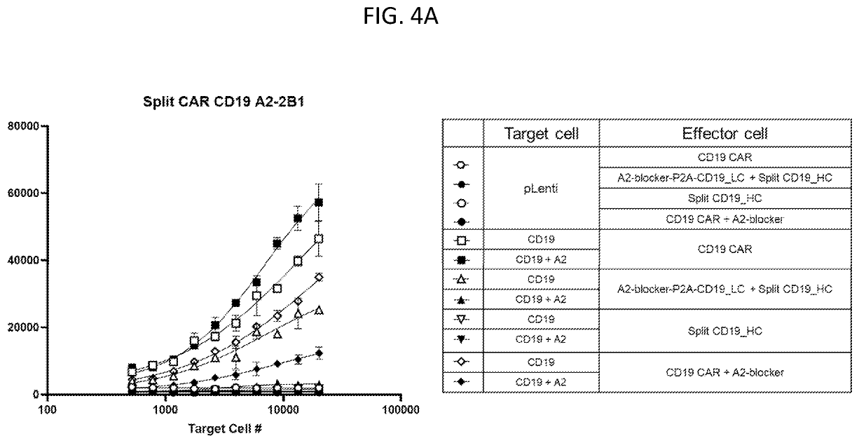

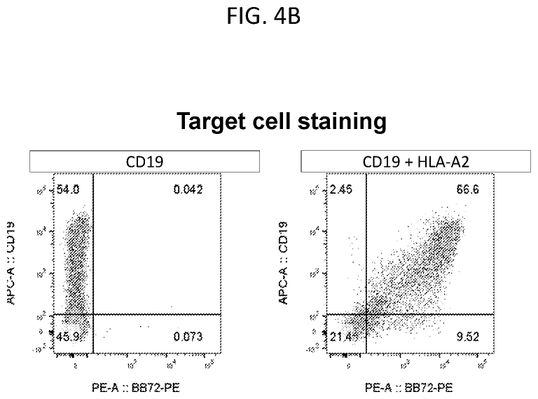

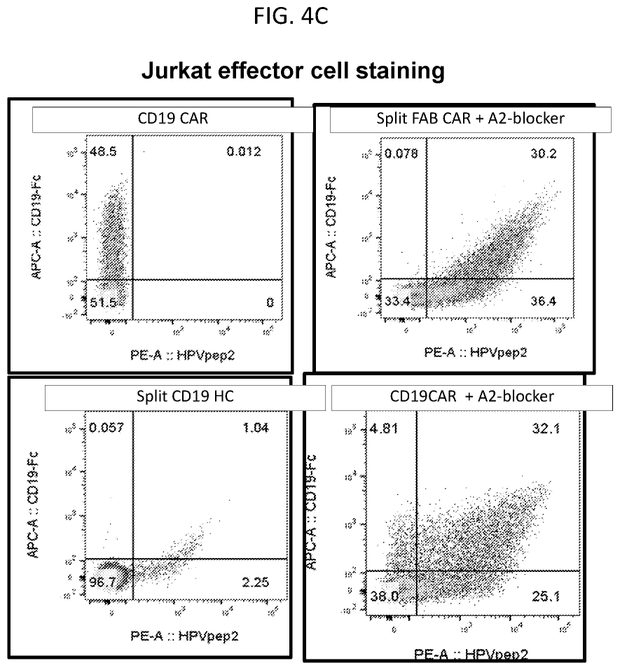

Jurkat NFAT Luciferase (JNL) reporter effector cells were transfected with the activator and blocker constructs disclosed in FIG. 4A. Sequences of the vectors used for the blocker and split activator combination are provided in Table 8 below. Cells were transfected with 1 ug of each construct per 1E6 cells, at 2 ug per 1E6 cells total. Cells were transfected using the Neon transfection system (100 uL volume, 1500 V, 10 ms, 3 pulses). Jurkat clone E6-1 cells were transfected with the target antigens using similar method as above except at 2 ug of each construct per 1E6 cells. 18-24 hours post-transfection, 10,000 effector Jurkat cells were co-cultured with increasing numbers of target Jurkat cells (FIG. 4A, left, X-axis), and activation was measured using the Luciferase reporter system.

As can be seen in FIG. 4A, Jurkat cells transfected with CD19 CAR split between the two vectors are activated in the presence of CD19+ target cells. When Jurkat cells are transfected with both the split CD19 activator CAR and the HLA-A*02 blocker using the dual vectors, the blocker is capable of inhibiting Jurkat activation in the presence of CD19+ HLA-A*02+ target cells. FIGS. 4B and 4C show that both the activator and blocker receptors are expressed by effector cells transformed with the dual vector system. Effector cells were stained with stained with fluorescently labeled HLA-A*02 probe to detect the blocker receptor, and Fc-tagged recombinant human CD19 (ACROBiosystems) to detect the activating receptor. Target cells were stained using anti-CD19 antibody and anti-HLA-A*02 antibody (Clone BB7.2).

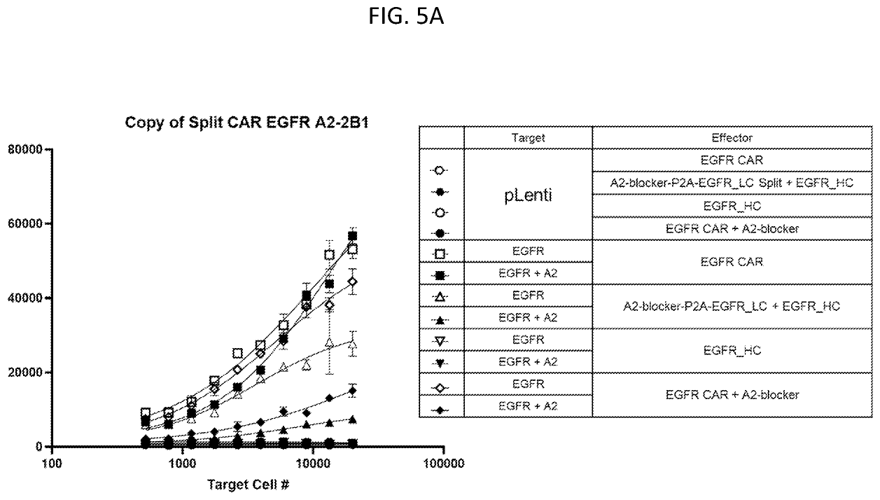

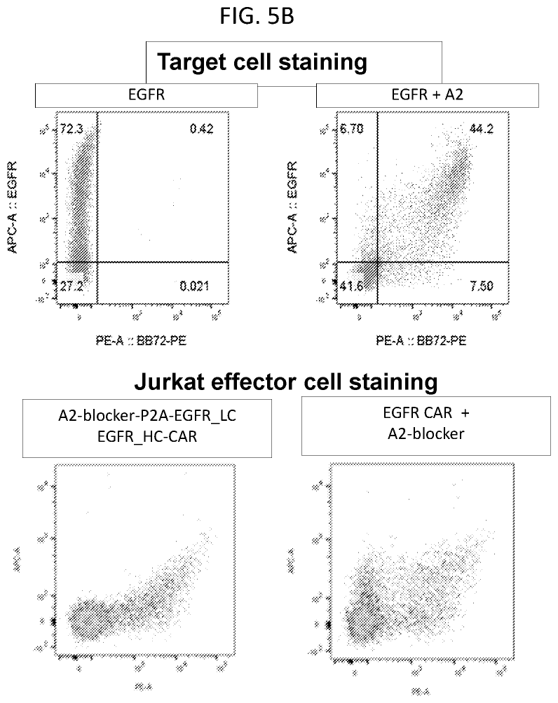

Similar results were obtained using an EGFR split activator CAR, with same arrangement shown as shown in FIG. 3. The results are shown in FIGS. 5A-5B. Jurkat NFAT Luciferase (JNL) reporter effector cells were transfected with the activator and blocker constructs disclosed in FIG. 5A. Cells were transfected 1 ug of each construct per 1E6 cells, at 2 ug per 1E6 cells total using the Neon transfection system (100 uL volume, 1500 V, 10 ms, 3 pulses). Jurkat clone E6-1 cells were transfected with the target antigens using similar method as above except at 2 ug of each construct per 1E6 cells. 18-24 hours post-transfection, 10000 effector Jurkat cells were co-cultured with increasing number of target Jurkat cells. FIG. 5B shows the expression of the EGFR activator and HLA-A*02 LIR1 blocker receptors by Jurkat effector cells (bottom row), as well as the expression of the EGFR and HLA-A*02 antigens by the target cells (top row). Effectors were stained with fluorescently labeled HLA-A*02 probe and recombinant human EGFR protein. Target cells were stained using anti-EGFR antibody and anti-HLA-A*02 antibody.

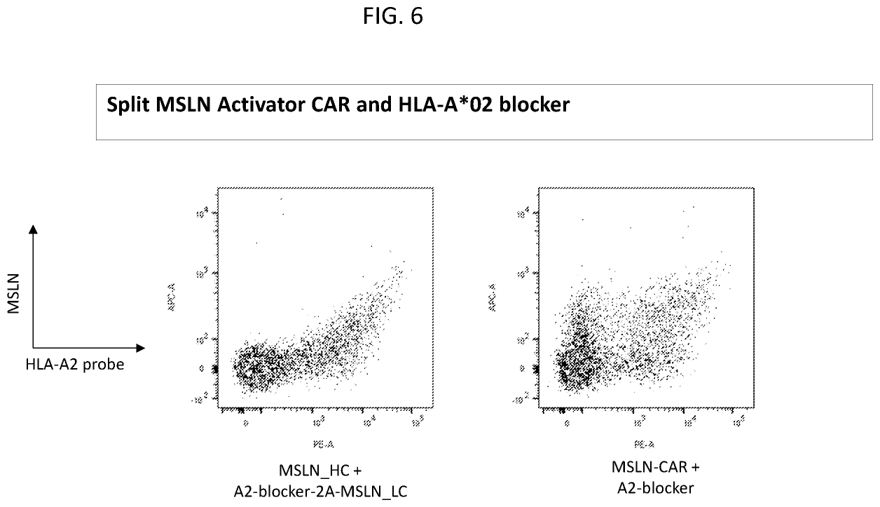

FIG. 6 shows that Jurkat NFAT Luciferase (JNL) reporter effector cells transfected with two vectors encoding a split MSLN CAR and an HLA-A*02 LIR1 blocker receptor, as shown in FIG. 3, expressed the MSLN CAR and HLA-A*02 LIR1 receptor after transfection. 1 day following transfection, cells were stained with biotinylated recombinant human MSLN protein (ACROBiosystems) or fluorescently labeled HLA-A*02 probe.

TABLE 7
Sequences of Constructs

| Name | DNA | Protein |
|---|---|---|
| HLA-A*02 | ATGGACATGAGGGTCCCCGCTCAGCTCCTGGGGCTCCTG | MDMRVPAQLLGLLL |
| ScFv | CTACTCTGGCTCCGAGGTGCCAGATGTCAGGTGCAGCTG | LWLRGARCQVQLVQ |
| | GTGCAGTCTGGGGCTGAGGTGAAGAAGCCTGGGTCCTCA | SGAEVKKPGSSVKV |
| | GTGAAGGTTTCCTGCAAGGCTTCTGGATACACCTTCACT | SCKASGYTFTSYHI |
| | AGCTATCATATACATTGGGTGCGCCAGGCCCCCGGACAA | HWVRQAPGQCLEWI |
| | TGTCTTGAGTGGATCGGATGGATCTACCCTGGCAATGTT | GWIYPGNVNTEYNE |
| | AACACAGAATATAATGAGAAGTTCAAGGGCAAAGCCACC | KFKGKATITADEST |
| | ATTACCGCGGACGAATCCACGAACACAGCCTACATGGAG | NTAYMELSSLRSED |
| | CTGAGCAGCCTGAGATCTGAAGACACGGCTGTGTATTAC | TAVYYCAREEITYA |
| | TGTGCGAGGGAGGAAATTACCTACGCTATGGACTACTGG | MDYWGQGTLVTVSS |
| | GGCCAGGGAACCCTGGTCACCGTGTCCTCAGGCGGAGGT | GGGGSGGGGSGGGG |
| | GGAAGCGGAGGGGGAGGATCTGGCGGCGGAGGAAGCGGA | SGGDIQMTQSPSTL |
| | GGCGACATTCAAATGACCCAGAGCCCATCCACCCTGAGC | SASVGDRVTITCRS |
| | GCATCTGTAGGTGACCGGGTCACCATCACTTGTAGATCC | SQSIVHSNGNTYLE |
| | AGTCAGAGTATTGTACACAGTAATGGGAACACCTATTTG | WYQQKPGKAPKLLI |
| | GAATGGTATCAGCAGAAACCAGGTAAAGCCCCAAAATTG | YKVSNRFSGVPARF |
| | CTCATCTACAAAGTCTCTAACAGATTTAGTGGTGTACCA | SGSGSGTEFTLTIS |
| | GCCAGGTTCAGCGGTTCCGGAAGTGGTACTGAATTCACC | SLQPDDFATYYCFQ |
| | CTCACGATCTCCTCTCTCCAGCCAGATGATTTCGCCACT | GSHVPRTFGCGTKV |
| | TATTACTGTTTTCAAGGTTCACATGTGCCGCGCACATTC | EVK |
| | GGTTGCGGTACTAAAGTAGAAGTCAAA | |
| LIR1 hinge + | TACGGCTCACAGAGCTCCAAACCCTACCTGCTGACTCAC | YGSQSSKPYLLTHP |
| TM + ICD | CCCAGTGACCCCCTGGAGCTCGTGGTCTCAGGACCGTCT | SDPLELVVSGPSGG |
| | GGGGGCCCCAGCTCCCCGACAACAGGCCCCACCTCCACA | PSSPTTGPTSTSGP |
| | TCTGGCCCTGAGGACCAGCCCCTCACCCCCACCGGGTCG | EDQPLTPTGSDPQS |
| | GATCCCCAGAGTGGTCTGGGAAGGCACCTGGGGGTTGTG | GLGRHLGVVIGILV |
| | ATCGGCATCTTGGTGGCCGTCATCCTACTGCTCCTCCTC | AVILLLLLLLLLFL |
| | CTCCTCCTCCTCTTCCTCATCCTCCGACATCGACGTCAG | ILRHRRQGKHWTST |
| | GGCAAACACTGGACATCGACCCAGAGAAAGGCTGATTTC | QRKADFQHPAGAVG |
| | CAACATCCTGCAGGGGCTGTGGGGCCAGAGCCCACAGAC | PEPTDRGLQWRSSP |
| | AGAGGCCTGCAGTGGAGGTCCAGCCCAGCTGCCGATGCC | AADAQEENLYAAVK |
| | CAGGAAGAAAACCTCTATGCTGCCGTGAAGCACACACAG | HTQPEDGVEMDTRS |
| | CCTGAGGATGGGGTGGAGATGGACACTCGGAGCCCACAC | PHDEDPQAVTYAEV |
| | GATGAAGACCCCCAGGCAGTGACGTATGCCGAGGTGAAA | KHSRPRREMASPPS |
| | CACTCCAGACCTAGGAGAGAAATGGCCTCTCCTCCTTCC | PLSGEFLDTKDRQA |
| | CCACTGTCTGGGGAATTCCTGGACACAAAGGACAGACAG | EEDRQMDTEAAASE |
| | GCGGAAGAGGACAGGCAGATGGACACTGAGGCTGCTGCA | APQDVTYAQLHSLT |
| | TCTGAAGCCCCCCAGGATGTGACCTACGCCCAGCTGCAC | LRREATEPPPSQEG |
| | AGCTTGACCCTCAGACGGGAGGCAACTGAGCCTCCTCCA | PSPAVPSIYATLAI |
| | TCCCAGGAAGGGCCCTCTCCAGCTGTGCCCAGCATCTAC | H |
| | GCCACTCTGGCCATCCAC | |
| P2A with N- | GGATCCGGAGCCACCAACTTCAGCCTGCTGAAGCAGGCC | GSGATNFSLLKQAG |
| terminal GSG | GGCGACGTGGAGGAGAACCCCGGCCCC | DVEENPGP |
| CD28 hinge + | TTCTGGGTGCTGGTCGTTGTGGGCGGCGTGCTGGCCTGC | FWVLVVVGGVLACY |
| TM, 4-1BB + | TACAGCCTGCTGGTGACAGTGGCCTTCATCATCTTTTGG | SLLVTVAFIIFWVR |
| CD3z ICD | GTGAGGAGCAAGCGGAGCAGACTGCTGCACAGCGACTAC | SKRSRLLHSDYMNM |
| (CD28 hinge + | ATGAACATGACCCCCCGGAGGCCTGGCCCCACCCGGAAG | TPRRPGPTRKHYQP |
| TM is | CACTACCAGCCCTACGCCCCTCCCAGGGATTTCGCCGCC | YAPPRDFAAYRSKR |
| bold, CD3z | TACCGGAGCAAACGGGGCAGAAAGAAACTCCTGTATATA | GRKKLLYIFKQPFM |
| is | TTCAAACAACCATTTATGAGGCCAGTACAAACTACTCAA | RPVQTTQEEDGCSC |
| underlined) | GAGGAAGATGGCTGTAGCTGCCGATTTCCAGAAGAAGAA | RFPEEEEGGCELRV |
| | GAAGGAGGATGTGAACTGAGAGTGAAGTTCAGCAGGAGC | KFSRSADAPAYKQG |
| | GCAGACGCCCCCGCGTACAAGCAGGGCCAGAACCAGCTC | QNQLYNELNLGRRE |
| | TATAACGAGCTCAATCTAGGACGAAGAGAGGAGTACGAT | EYDVLDKRRGRDPE |
| | GTTTTGGACAAGCGTAGAGGCCGGGACCCTGAGATGGGG | MGGKPRRKNPQEGL |
| | GGAAAGCCGAGAAGGAAGAACCCTCAGGAAGGCCTGTAC | YNELQKDKMAEAYS |
| | AATGAACTGCAGAAAGATAAGATGGCGGAGGCCTACAGT | EIGMKGERRRGKGH |
| | GAGATTGGGATGAAAGGCGAGCGCCGGAGGGGCAAGGGG | DGLYQGLSTATKDT |
| | CACGATGGCCTTTACCAGGGACTCAGTACAGCCACCAAG | YDALHMQALPPR |
| | GACACCTACGACGCCCTTCACATGCAGGCCCTGCCCCCT | |
| | CGC | |
| LC constant | CGGACCGTGGCCGCTCCCAGCGTGTTCATCTTCCCCCCC | RTVAAPSVFIFPPS |
| region | AGCGACGAGCAGCTTAAGAGCGGTACCGCTAGCGTGGTG | DEQLKSGTASVVCL |
| | TGCCTGCTGAACAACTTCTACCCCCGGGAGGCCAAGGTG | LNNFYPREAKVQWK |
| | CAGTGGAAGGTGGACAACGCCCTGCAGAGCGGCAACAGC | VDNALQSGNSQESV |
| | CAGGAAAGCGTCACCGAGCAGGACAGCAAGGACTCCACC | TEQDSKDSTYSLSS |
| | TACAGCCTGAGCAGCACCCTGACCCTGAGCAAGGCCGAC | TLTLSKADYEKHKV |
| | TACGAGAAGCACAAGGTGTACGCCTGCGAAGTGACCCAC | YACEVTHQGLSSPV |
| | CAGGGCCTGTCCAGCCCCGTGACCAAGAGCTTCAACCGG | TKSFNRGEC |
| | GGCGAGTGC | |
| HC constant | TGGGGCCAAGGAACCTCAGTCACCGTGTCCTCAGCCTCC | WGQGTSVTVSSAST |
| region | ACCAAGGGCCCATCGGTCTTCCCCCTGGCACCCTCCTCC | KGPSVFPLAPSSKS |
| | AAGAGCACCTCTGGGGGCACAGCGGCCCTGGGCTGCCTG | TSGGTAALGCLVKD |
| | GTCAAGGACTACTTCCCCGAACCGGTGACGGTGTCGTGG | YFPEPVTVSWNSGA |
| | AACTCAGGCGCCCTGACCAGCGGCGTGCATACCTTCCCG | LTSGVHTFPAVLQS |
| | GCTGTCCTACAGTCCTCAGGACTCTACTCCCTCAGCAGC | SGLYSLSSVVTVPS |
| | GTGGTGACCGTGCCCTCCAGCAGCTTGGGCACCCAGACC | SSLGTQTYICNVNH |
| | TACATCTGCAACGTGAATCACAAGCCCAGCAACACCAAG | KPSNTKVDKKVEPK |
| | GTGGACAAGAAAGTTGAGCCCAAATCTTGTACCACGACG | SCTTTPAPRPPTPA |
| | CCAGCGCCGCGACCACCAACACCGGCGCCCACCATCGCG | PTIASQPLSLRPEA |
| | TCGCAGCCCCTGTCCCTGCGCCCAGAGGCGTGCCGGCCA | CRPAAGGAVHTRGL |
| | GCGGCGGGGGGCGCAGTGCACACGAGGGGGCTGGACTTC | DFACD |
| | GCCTGTGAT | |
| HC constant | TGGGGTCAAGGTACCTCAGTCACCGTGTCCTCAGCCTCC | WGQGTSVTVSSAST |
| region, | ACCAAGGGCCCATCGGTCTTCCCCCTGGCACCCTCCTCC | KGPSVFPLAPSSKS |
| alternative | AAGAGCACCTCTGGGGGCACAGCGGCCCTGGGCTGCCTG | TSGGTAALGCLVKD |
| DNA sequence | GTCAAGGACTACTTCCCCGAACCGGTGACGGTGTCGTGG | YFPEPVTVSWNSGA |
| | AACTCAGGCGCCCTGACCAGCGGCGTGCATACCTTCCCG | LTSGVHTFPAVLQS |
| | GCTGTCCTACAGTCCTCAGGACTCTACTCCCTCAGCAGC | SGLYSLSSVVTVPS |
| | GTGGTGACCGTGCCCTCCAGCAGCTTGGGCACCCAGACC | SSLGTQTYICNVNH |
| | TACATCTGCAACGTGAATCACAAGCCCAGCAACACCAAG | KPSNTKVDKKVEPK |
| | GTGGACAAGAAAGTTGAGCCCAAATCTTGTACCACGACG | SCTTTPAPRPPTPA |
| | CCAGCGCCGCGACCACCAACACCGGCGCCCACCATCGCG | PTIASQPLSLRPEA |
| | TCGCAGCCCCTGTCCCTGCGCCCAGAGGCGTGCCGGCCA | CRPAAGGAVHTRGL |
| | GCGGCGGGGGGCGCAGTGCACACGAGGGGGCTGGACTTC | DFACD |
| | GCCTGTGAT | |
| HC constant | TGGGGCCAGGGAACCCTGGTCACCGTGTCCTCAGCCTCC | WGQGTLVTVSSAST |
| region, | ACCAAGGGCCCATCGGTCTTCCCCCTGGCACCCTCCTCC | KGPSVFPLAPSSKS |
| alternative | AAGAGCACCTCTGGGGGCACAGCGGCCCTGGGCTGCCTG | TSGGTAALGCLVKD |
| sequence | GTCAAGGACTACTTCCCCGAACCGGTGACGGTGTCGTGG | YFPEPVTVSWNSGA |
| | AACTCAGGCGCCCTGACCAGCGGCGTGCATACCTTCCCG | LTSGVHTFPAVLQS |
| | GCTGTCCTACAGTCCTCAGGACTCTACTCCCTCAGCAGC | SGLYSLSSVVTVPS |
| | GTGGTGACCGTGCCCTCCAGCAGCTTGGGCACCCAGACC | SSLGTQTYICNVNH |
| | TACATCTGCAACGTGAATCACAAGCCCAGCAACACCAAG | KPSNTKVDKKVEPK |
| | GTGGACAAGAAAGTTGAGCCCAAATCTTGTACCACGACG | SCTTTPAPRPPTPA |
| | CCAGCGCCGCGACCACCAACACCGGCGCCCACCATCGCG | PTIASQPLSLRPEA |
| | TCGCAGCCCCTGTCCCTGCGCCCAGAGGCGTGCCGGCCA | CRPAAGGAVHTRGL |
| | GCGGCGGGGGGCGCAGTGCACACGAGGGGGCTGGACTTC | DFACD |
| | GCCTGTGAT | |
| Anti-CD19 LC | ATGCTTCTCCTGGTGACAAGCCTTCTGCTCTGTGAGTTA | MLLLVTSLLLCELP |
| variable | CCACACCCAGCATTCCTCCTGATCCCAGACATCCAGATG | HPAFLLIPDIQMTQ |
| region | ACACAGACTACATCCTCCCTGTCTGCCTCTCTGGGAGAC | TTSSLSASLGDRVT |
| | AGAGTCACCATCAGTTGCAGGGCAAGTCAGGACATTAGT | ISCRASQDISKYLN |
| | AAATATTTAAATTGGTATCAGCAGAAACCAGATGGAACT | WYQQKPDGTVKLLI |
| | GTTAAACTCCTGATCTACCATACATCAAGATTACACTCA | YHTSRLHSGVPSRF |
| | GGAGTCCCATCAAGGTTCAGTGGCAGTGGGTCTGGAACA | SGSGSGTDYSLTIS |
| | GATTATTCTCTCACCATTAGCAACCTGGAGCAAGAAGAT | NLEQEDIATYFCQQ |
| | ATTGCCACTTACTTTTGCCAACAGGGTAATACGCTTCCG | GNTLPYTFGGGTKL |
| | TACACGTTCGGAGGGGGGACCAAGCTGGAGATCACACTT | EITLLLVTSLLLCE |
| | CTCCTGGTGACAAGCCTTCTGCTCTGTGAGTTACCACAC | LPHPAFLLIPDIQM |
| | CCAGCATTCCTCCTGATCCCAGACATCCAGATGACACAG | TQTTSSLSASLGDR |
| | ACTACATCCTCCCTGTCTGCCTCTCTGGGAGACAGAGTC | VTISCRASQDISKY |
| | ACCATCAGTTGCAGGGCAAGTCAGGACATTAGTAAATAT | LNWYQQKPDGTVKL |
| | TTAAATTGGTATCAGCAGAAACCAGATGGAACTGTTAAA | LIYHTSRLHSGVPS |
| | CTCCTGATCTACCATACATCAAGATTACACTCAGGAGTC | RFSGSGSGTDYSLT |
| | CCATCAAGGTTCAGTGGCAGTGGGTCTGGAACAGATTAT | ISNLEQEDIATYFC |
| | TCTCTCACCATTAGCAACCTGGAGCAAGAAGATATTGCC | QQGNTLPYTFGGGT |
| | ACTTACTTTTGCCAACAGGGTAATACGCTTCCGTACACG | KLEIT |
| | TTCGGAGGGGGGACCAAGCTGGAGATCACA | |
| Anti-CD19 LC | ATGCTTCTCCTGGTGACAAGCCTTCTGCTCTGTGAGTTA | MLLLVTSLLLCELP |
| (variable | CCACACCCAGCATTCCTCCTGATCCCAGACATCCAGATG | HPAFLLIPDIQMTQ |
| region is | ACACAGACTACATCCTCCCTGTCTGCCTCTCTGGGAGAC | TTSSLSASLGDRVT |
| underlined) | AGAGTCACCATCAGTTGCAGGGCAAGTCAGGACATTAGT | ISCRASQDISKYLN |
| | AAATATTTAAATTGGTATCAGCAGAAACCAGATGGAACT | WYQQKPDGTVKLLI |
| | GTTAAACTCCTGATCTACCATACATCAAGATTACACTCA | YHTSRLHSGVPSRF |
| | GGAGTCCCATCAAGGTTCAGTGGCAGTGGGTCTGGAACA | SGSGSGTDYSLTIS |
| | GATTATTCTCTCACCATTAGCAACCTGGAGCAAGAAGAT | NLEQEDIATYFCQQ |
| | ATTGCCACTTACTTTTGCCAACAGGGTAATACGCTTCCG | GNTLPYTFGGGTKL |
| | TACACGTTCGGAGGGGGGACCAAGCTGGAGATCACACTT | EITLLLVTSLLLCE |
| | CTCCTGGTGACAAGCCTTCTGCTCTGTGAGTTACCACAC | LPHPAFLLIPDIQM |
| | CCAGCATTCCTCCTGATCCCAGACATCCAGATGACACAG | TQTTSSLSASLGDR |
| | ACTACATCCTCCCTGTCTGCCTCTCTGGGAGACAGAGTC | VTISCRASQDISKY |
| | ACCATCAGTTGCAGGGCAAGTCAGGACATTAGTAAATAT | LNWYQQKPDGTVKL |
| | TTAAATTGGTATCAGCAGAAACCAGATGGAACTGTTAAA | LIYHTSRLHSGVPS |
| | CTCCTGATCTACCATACATCAAGATTACACTCAGGAGTC | RFSGSGSGTDYSLT |
| | CCATCAAGGTTCAGTGGCAGTGGGTCTGGAACAGATTAT | ISNLEQEDIATYFC |
| | TCTCTCACCATTAGCAACCTGGAGCAAGAAGATATTGCC | QQGNTLPYTFGGGT |
| | ACTTACTTTTGCCAACAGGGTAATACGCTTCCGTACACG | KLEITRTVAAPSVF |
| | TTCGGAGGGGGGACCAAGCTGGAGATCACACGGACCGTG | IFPPSDEQLKSGTA |
| | GCCGCTCCCAGCGTGTTCATCTTCCCCCCCAGCGACGAG | SVVCLLNNFYPREA |
| | CAGCTTAAGAGCGGTACCGCTAGCGTGGTGTGCCTGCTG | KVQWKVDNALQSGN |
| | AACAACTTCTACCCCCGGGAGGCCAAGGTGCAGTGGAAG | SQESVTEQDSKDST |
| | GTGGACAACGCCCTGCAGAGCGGCAACAGCCAGGAAAGC | YSLSSTLTLSKADY |
| | GTCACCGAGCAGGACAGCAAGGACTCCACCTACAGCCTG | EKHKVYACEVTHQG |
| | AGCAGCACCCTGACCCTGAGCAAGGCCGACTACGAGAAG | LSSPVTKSFNRGEC |
| | CACAAGGTGTACGCCTGCGAAGTGACCCACCAGGGCCTG | |
| | TCCAGCCCCGTGACCAAGAGCTTCAACCGGGGCGAGTGC | |
| Anti-CD19 HC | ATGGACATGAGGGTCCCCGCTCAGCTCCTGGGGCTCCTG | MDMRVPAQLLGLLL |
| variable | CTACTCTGGCTCCGAGGTGCCAGATGTGAGGTGAAACTG | LWLRGARCEVKLQE |
| region | CAGGAGTCAGGACCTGGCCTGGTGGCGCCCTCACAGAGC | SGPGLVAPSQSLSV |
| | CTGTCCGTCACATGCACTGTCTCAGGGGTCTCATTACCC | TCTVSGVSLPDYGV |
| | GACTATGGTGTAAGCTGGATTCGCCAGCCTCCACGAAAG | SWIRQPPRKGLEWL |
| | GGTCTGGAGTGGCTGGGAGTAATATGGGGTAGTGAAACC | GVIWGSETTYYNSA |
| | ACATACTATAATTCAGCTCTCAAATCCAGACTGACCATC | LKSRLTIIKDNSKS |
| | ATCAAGGACAACTCCAAGAGCCAAGTTTTCTTAAAAATG | QVFLKMNSLQTDDT |
| | AACAGTCTGCAAACTGATGACACAGCCATTTACTACTGT | AIYYCAKHYYYGGS |
| | GCCAAACATTATTACTACGGTGGTAGCTATGCTATGGAC | YAMDY |
| | TAC | |
| Anti-CD19 HC | ATGGACATGAGGGTCCCCGCTCAGCTCCTGGGGCTCCTG | MDMRVPAQLLGLLL |
| (variable | CTACTCTGGCTCCGAGGTGCCAGATGTGAGGTGAAACTG | LWLRGARCEVKLQE |
| region is | CAGGAGTCAGGACCTGGCCTGGTGGCGCCCTCACAGAGC | SGPGLVAPSQSLSV |
| underlined) | CTGTCCGTCACATGCACTGTCTCAGGGGTCTCATTACCC | TCTVSGVSLPDYGV |
| | GACTATGGTGTAAGCTGGATTCGCCAGCCTCCACGAAAG | SWIRQPPRKGLEWL |
| | GGTCTGGAGTGGCTGGGAGTAATATGGGGTAGTGAAACC | GVIWGSETTYYNSA |
| | ACATACTATAATTCAGCTCTCAAATCCAGACTGACCATC | LKSRLTIIKDNSKS |
| | ATCAAGGACAACTCCAAGAGCCAAGTTTTCTTAAAAATG | QVFLKMNSLQTDDT |
| | AACAGTCTGCAAACTGATGACACAGCCATTTACTACTGT | AIYYCAKHYYYGGS |
| | GCCAAACATTATTACTACGGTGGTAGCTATGCTATGGAC | YAMDYWGQGTSVTV |
| | TACTGGGGCCAAGGAACCTCAGTCACCGTGTCCTCAGCC | SSASTKGPSVFPLA |
| | TCCACCAAGGGCCCATCGGTCTTCCCCCTGGCACCCTCC | PSSKSTSGGTAALG |
| | TCCAAGAGCACCTCTGGGGGCACAGCGGCCCTGGGCTGC | CLVKDYFPEPVTVS |
| | CTGGTCAAGGACTACTTCCCCGAACCGGTGACGGTGTCG | WNSGALTSGVHTFP |
| | TGGAACTCAGGCGCCCTGACCAGCGGCGTGCATACCTTC | AVLQSSGLYSLSSV |
| | CCGGCTGTCCTACAGTCCTCAGGACTCTACTCCCTCAGC | VTVPSSSLGTQTYI |
| | AGCGTGGTGACCGTGCCCTCCAGCAGCTTGGGCACCCAG | CNVNHKPSNTKVDK |
| | ACCTACATCTGCAACGTGAATCACAAGCCCAGCAACACC | KVEPKSCTTTPAPR |
| | AAGGTGGACAAGAAAGTTGAGCCCAAATCTTGTACCACG | PPTPAPTIASQPLS |
| | ACGCCAGCGCCGCGACCACCAACACCGGCGCCCACCATC | LRPEACRPAAGGAV |
| | GCGTCGCAGCCCCTGTCCCTGCGCCCAGAGGCGTGCCGG | HTRGLDFACD |
| | CCAGCGGCGGGGGGCGCAGTGCACACGAGGGGGCTGGAC | |
| | TTCGCCTGTGAT | |

TABLE 8
Full Length Constructs

| Name | DNA Sequence | Protein Sequence |
|---|---|---|
| HLA-A*02 | ATGGACATGAGGGTCCCCGCTCAGCTCCTGGGGCTCCTG | MDMRVPAQLLGLLL |
| LIR1 blocker | CTACTCTGGCTCCGAGGTGCCAGATGTCAGGTGCAGCTG | LWLRGARCQVQLVQ |
| receptor, | GTGCAGTCTGGGGCTGAGGTGAAGAAGCCTGGGTCCTCA | SGAEVKKPGSSVKV |
| P2A, anti- | GTGAAGGTTTCCTGCAAGGCTTCTGGATACACCTTCACT | SCKASGYTFTSYHI |
| CD19 light | AGCTATCATATACATTGGGTGCGCCAGGCCCCCGGACAA | HWVRQAPGQCLEWI |
| chain | TGTCTTGAGTGGATCGGATGGATCTACCCTGGCAATGTT | GWIYPGNVNTEYNE |
| | AACACAGAATATAATGAGAAGTTCAAGGGCAAAGCCACC | KFKGKATITADEST |
| | ATTACCGCGGACGAATCCACGAACACAGCCTACATGGAG | NTAYMELSSLRSED |
| | CTGAGCAGCCTGAGATCTGAAGACACGGCTGTGTATTAC | TAVYYCAREEITYA |
| | TGTGCGAGGGAGGAAATTACCTACGCTATGGACTACTGG | MDYWGQGTLVTVSS |
| | GGCCAGGGAACCCTGGTCACCGTGTCCTCAGGCGGAGGT | GGGGSGGGGSGGGG |
| | GGAAGCGGAGGGGGAGGATCTGGCGGCGGAGGAAGCGGA | SGGDIQMTQSPSTL |
| | GGCGACATTCAAATGACCCAGAGCCCATCCACCCTGAGC | SASVGDRVTITCRS |
| | GCATCTGTAGGTGACCGGGTCACCATCACTTGTAGATCC | SQSIVHSNGNTYLE |
| | AGTCAGAGTATTGTACACAGTAATGGGAACACCTATTTG | WYQQKPGKAPKLLI |
| | GAATGGTATCAGCAGAAACCAGGTAAAGCCCCAAAATTG | YKVSNRFSGVPARF |
| | CTCATCTACAAAGTCTCTAACAGATTTAGTGGTGTACCA | SGSGSGTEFTLTIS |
| | GCCAGGTTCAGCGGTTCCGGAAGTGGTACTGAATTCACC | SLQPDDFATYYCFQ |
| | CTCACGATCTCCTCTCTCCAGCCAGATGATTTCGCCACT | GSHVPRTFGCGTKV |
| | TATTACTGTTTTCAAGGTTCACATGTGCCGCGCACATTC | EVKYGSQSSKPYLL |
| | GGTTGCGGTACTAAAGTAGAAGTCAAATACGGCTCACAG | THPSDPLELVVSGP |
| | AGCTCCAAACCCTACCTGCTGACTCACCCCAGTGACCCC | SGGPSSPTTGPTST |
| | CTGGAGCTCGTGGTCTCAGGACCGTCTGGGGGCCCCAGC | SGPEDQPLTPTGSD |
| | TCCCCGACAACAGGCCCCACCTCCACATCTGGCCCTGAG | PQSGLGRHLGVVIG |
| | GACCAGCCCCTCACCCCCACCGGGTCGGATCCCCAGAGT | ILVAVILLLLLLLL |
| | GGTCTGGGAAGGCACCTGGGGGTTGTGATCGGCATCTTG | LFLILRHRRQGKHW |
| | GTGGCCGTCATCCTACTGCTCCTCCTCCTCCTCCTCCTC | TSTQRKADFQHPAG |
| | TTCCTCATCCTCCGACATCGACGTCAGGGCAAACACTGG | AVGPEPTDRGLQWR |
| | ACATCGACCCAGAGAAAGGCTGATTTCCAACATCCTGCA | SSPAADAQEENLYA |
| | GGGGCTGTGGGGCCAGAGCCCACAGACAGAGGCCTGCAG | AVKHTQPEDGVEMD |
| | TGGAGGTCCAGCCCAGCTGCCGATGCCCAGGAAGAAAAC | TRSPHDEDPQAVTY |
| | CTCTATGCTGCCGTGAAGCACACACAGCCTGAGGATGGG | AEVKHSRPRREMAS |
| | GTGGAGATGGACACTCGGAGCCCACACGATGAAGACCCC | PPSPLSGEFLDTKD |
| | CAGGCAGTGACGTATGCCGAGGTGAAACACTCCAGACCT | RQAEEDRQMDTEAA |
| | AGGAGAGAAATGGCCTCTCCTCCTTCCCCACTGTCTGGG | ASEAPQDVTYAQLH |
| | GAATTCCTGGACACAAAGGACAGACAGGCGGAAGAGGAC | SLTLRREATEPPPS |
| | AGGCAGATGGACACTGAGGCTGCTGCATCTGAAGCCCCC | QEGPSPAVPSIYAT |
| | CAGGATGTGACCTACGCCCAGCTGCACAGCTTGACCCTC | LAIHGSGATNFSLL |
| | AGACGGGAGGCAACTGAGCCTCCTCCATCCCAGGAAGGG | KQAGDVEENPGPML |
| | CCCTCTCCAGCTGTGCCCAGCATCTACGCCACTCTGGCC | LLVTSLLLCELPHP |
| | ATCCACGGATCCGGAGCCACCAACTTCAGCCTGCTGAAG | AFLLIPDIQMTQTT |
| | CAGGCCGGCGACGTGGAGGAGAACCCCGGCCCCATGCTT | SSLSASLGDRVTIS |
| | CTCCTGGTGACAAGCCTTCTGCTCTGTGAGTTACCACAC | CRASQDISKYLNWY |
| | CCAGCATTCCTCCTGATCCCAGACATCCAGATGACACAG | QQKPDGTVKLLIYH |
| | ACTACATCCTCCCTGTCTGCCTCTCTGGGAGACAGAGTC | TSRLHSGVPSRFSG |
| | ACCATCAGTTGCAGGGCAAGTCAGGACATTAGTAAATAT | SGSGTDYSLTISNL |
| | TTAAATTGGTATCAGCAGAAACCAGATGGAACTGTTAAA | EQEDIATYFCQQGN |
| | CTCCTGATCTACCATACATCAAGATTACACTCAGGAGTC | TLPYTFGGGTKLEI |
| | CCATCAAGGTTCAGTGGCAGTGGGTCTGGAACAGATTAT | TRTVAAPSVFIFPP |
| | TCTCTCACCATTAGCAACCTGGAGCAAGAAGATATTGCC | SDEQLKSGTASVVC |
| | ACTTACTTTTGCCAACAGGGTAATACGCTTCCGTACACG | LLNNFYPREAKVQW |
| | TTCGGAGGGGGGACCAAGCTGGAGATCACACGGACCGTG | KVDNALQSGNSQES |
| | GCCGCTCCCAGCGTGTTCATCTTCCCCCCCAGCGACGAG | VTEQDSKDSTYSLS |
| | CAGCTTAAGAGCGGTACCGCTAGCGTGGTGTGCCTGCTG | STLTLSKADYEKHK |
| | AACAACTTCTACCCCCGGGAGGCCAAGGTGCAGTGGAAG | VYACEVTHQGLSSP |
| | GTGGACAACGCCCTGCAGAGCGGCAACAGCCAGGAAAGC | VTKSFNRGEC |
| | GTCACCGAGCAGGACAGCAAGGACTCCACCTACAGCCTG | |
| | AGCAGCACCCTGACCCTGAGCAAGGCCGACTACGAGAAG | |
| | CACAAGGTGTACGCCTGCGAAGTGACCCACCAGGGCCTG | |
| | TCCAGCCCCGTGACCAAGAGCTTCAACCGGGGCGAGTGC | |
| Anti-CD19 | ATGGACATGAGGGTCCCCGCTCAGCTCCTGGGGCTCCTG | MDMRVPAQLLGLLL |
| Heavy Chain | CTACTCTGGCTCCGAGGTGCCAGATGTGAGGTGAAACTG | LWLRGARCEVKLQE |
| CAR (CD28, | CAGGAGTCAGGACCTGGCCTGGTGGCGCCCTCACAGAGC | SGPGLVAPSQSLSV |
| 4-1BB, CD3z) | CTGTCCGTCACATGCACTGTCTCAGGGGTCTCATTACCC | TCTVSGVSLPDYGV |
| | GACTATGGTGTAAGCTGGATTCGCCAGCCTCCACGAAAG | SWIRQPPRKGLEWL |
| | GGTCTGGAGTGGCTGGGAGTAATATGGGGTAGTGAAACC | GVIWGSETTYYNSA |
| | ACATACTATAATTCAGCTCTCAAATCCAGACTGACCATC | LKSRLTIIKDNSKS |
| | ATCAAGGACAACTCCAAGAGCCAAGTTTTCTTAAAAATG | QVFLKMNSLQTDDT |
| | AACAGTCTGCAAACTGATGACACAGCCATTTACTACTGT | AIYYCAKHYYYGGS |
| | GCCAAACATTATTACTACGGTGGTAGCTATGCTATGGAC | YAMDYWGQGTSVTV |
| | TACTGGGGCCAAGGAACCTCAGTCACCGTGTCCTCAGCC | SSASTKGPSVFPLA |
| | TCCACCAAGGGCCCATCGGTCTTCCCCCTGGCACCCTCC | PSSKSTSGGTAALG |
| | TCCAAGAGCACCTCTGGGGGCACAGCGGCCCTGGGCTGC | CLVKDYFPEPVTVS |
| | CTGGTCAAGGACTACTTCCCCGAACCGGTGACGGTGTCG | WNSGALTSGVHTFP |
| | TGGAACTCAGGCGCCCTGACCAGCGGCGTGCATACCTTC | AVLQSSGLYSLSSV |
| | CCGGCTGTCCTACAGTCCTCAGGACTCTACTCCCTCAGC | VTVPSSSLGTQTYI |
| | AGCGTGGTGACCGTGCCCTCCAGCAGCTTGGGCACCCAG | CNVNHKPSNTKVDK |
| | ACCTACATCTGCAACGTGAATCACAAGCCCAGCAACACC | KVEPKSCTTTPAPR |
| | AAGGTGGACAAGAAAGTTGAGCCCAAATCTTGTACCACG | PPTPAPTIASQPLS |
| | ACGCCAGCGCCGCGACCACCAACACCGGCGCCCACCATC | LRPEACRPAAGGAV |
| | GCGTCGCAGCCCCTGTCCCTGCGCCCAGAGGCGTGCCGG | HTRGLDFACDFWVL |
| | CCAGCGGCGGGGGGCGCAGTGCACACGAGGGGGCTGGAC | VVVGGVLACYSLLV |
| | TTCGCCTGTGATTTCTGGGTGCTGGTCGTTGTGGGCGGC | TVAFIIFWVRSKRS |
| | GTGCTGGCCTGCTACAGCCTGCTGGTGACAGTGGCCTTC | RLLHSDYMNMTPRR |
| | ATCATCTTTTGGGTGAGGAGCAAGCGGAGCAGACTGCTG | PGPTRKHYQPYAPP |
| | CACAGCGACTACATGAACATGACCCCCCGGAGGCCTGGC | RDFAAYRSKRGRKK |
| | CCCACCCGGAAGCACTACCAGCCCTACGCCCCTCCCAGG | LLYIFKQPFMRPVQ |
| | GATTTCGCCGCCTACCGGAGCAAACGGGGCAGAAAGAAA | TTQEEDGCSCREPE |
| | CTCCTGTATATATTCAAACAACCATTTATGAGGCCAGTA | EEEGGCELRVKFSR |
| | CAAACTACTCAAGAGGAAGATGGCTGTAGCTGCCGATTT | SADAPAYKQGQNQL |
| | CCAGAAGAAGAAGAAGGAGGATGTGAACTGAGAGTGAAG | YNELNLGRREEYDV |
| | TTCAGCAGGAGCGCAGACGCCCCCGCGTACAAGCAGGGC | LDKRRGRDPEMGGK |
| | CAGAACCAGCTCTATAACGAGCTCAATCTAGGACGAAGA | PRRKNPQEGLYNEL |
| | GAGGAGTACGATGTTTTGGACAAGCGTAGAGGCCGGGAC | QKDKMAEAYSEIGM |
| | CCTGAGATGGGGGGAAAGCCGAGAAGGAAGAACCCTCAG | KGERRRGKGHDGLY |
| | GAAGGCCTGTACAATGAACTGCAGAAAGATAAGATGGCG | QGLSTATKDTYDAL |
| | GAGGCCTACAGTGAGATTGGGATGAAAGGCGAGCGCCGG | HMQALPPR |
| | AGGGGCAAGGGGCACGATGGCCTTTACCAGGGACTCAGT | |
| | ACAGCCACCAAGGACACCTACGACGCCCTTCACATGCAG | |
| | GCCCTGCCCCCTCGC | |

### Example 2: Splitting the Activator Receptor at the Hinge and/or Transmembrane Domains

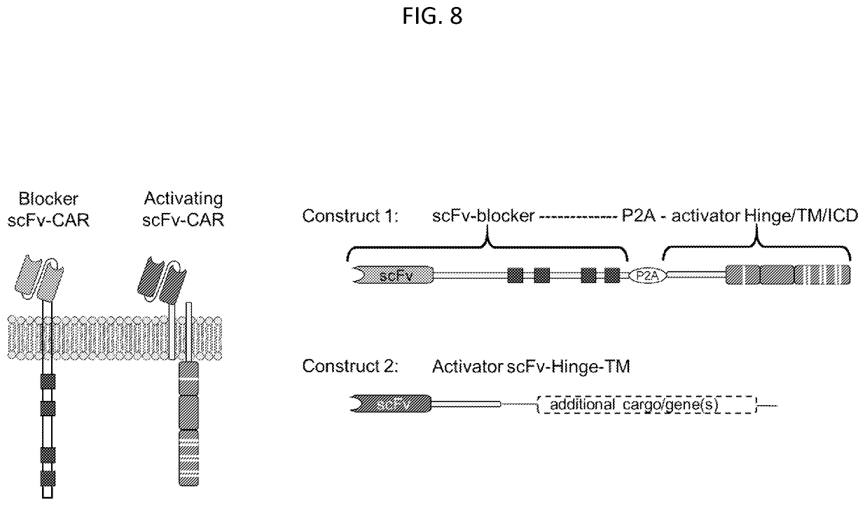

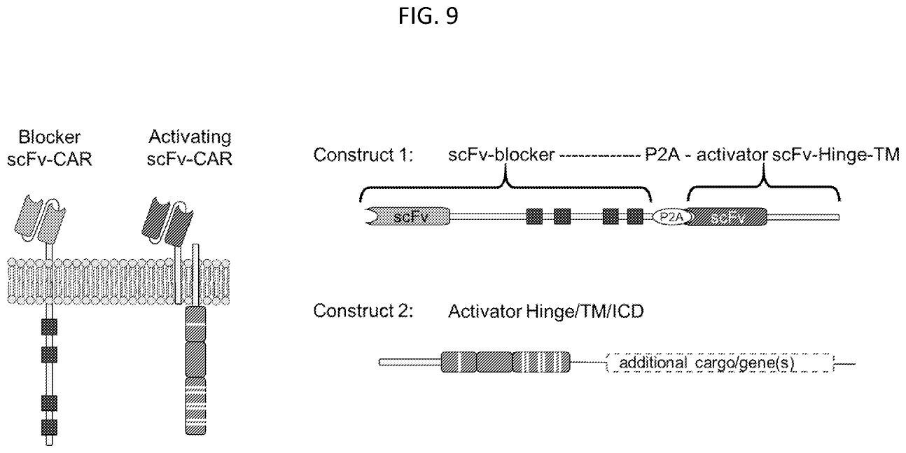

Instead of splitting via the antigen binding domain, the activator receptor can also be split at the hinge/transmembrane domains. In this case, one part of the activator receptor has a complete antigen binding domain, as well as hinge and transmembrane domains, while the other part of the receptor has the hinge, transmembrane and intracellular domains. Interactions mediated by the hinge, or hinge and transmembrane domains, lead to the reconstitution of a functional receptor when both parts of the receptor are expressed by a cell. Examples of this split receptor are show in FIGS. 8, 9 and 12B (top row).

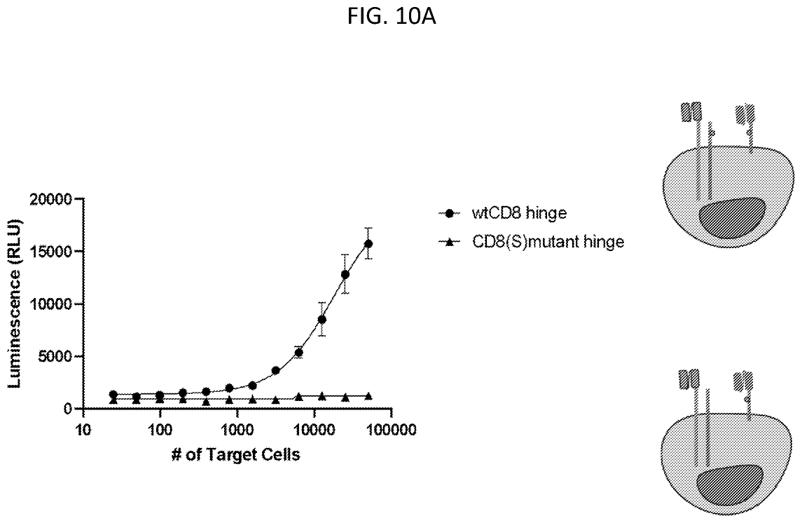

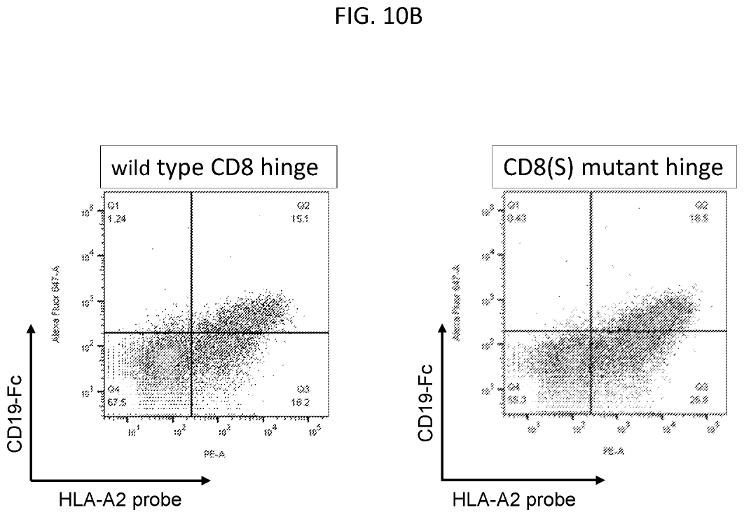

In one case, dimerization mediated by the CD8α hinge domain is sufficient to reconstitute a functional activator when the two parts of the activator shown in FIG. 8 were expressed by a cell. As can be seen in FIG. 10A, association of the CD8α hinges is sufficient to produce a functional activator receptor. This interaction is mediated by the cysteines in the hinge, as when the cysteines are mutated activator function is abolished.

FIG. 10A shows the results when the cysteines in CD8α hinge region of the activator CAR were mutated to serines (CD8(S) mutant hinge), and the function of the split CAR with and without the mutated hinge was compared in an NFAT-Luciferase assay. Jurkat NFAT-Luciferase effector cells were transfected with a split CD19 CAR construct containing either a wild type CD8 hinge (closed circle) or mutated CD8α hinge (triangle) as shown in FIG. 10A. Sequences are provided in Table 9 below. Jurkat NFAT-luciferase cells were transfected using Neon transfection (1500 V, 10 ms, 3 pulses). Jurkat clone E6-1 cells were transfected to express CD19 antigen using similar methods, except that 2 ug of each construct per 1E6 cells were used. 18-24 hours post-transfection, 10000 effector Jurkat cells were co-cultured with increasing numbers of target Jurkat cells in a cell titration assay. Expression of the receptors was measured by staining with human CD19-Fc fusion protein and fluorescently labeled HLA-A*02 probe (FIG. 10B).

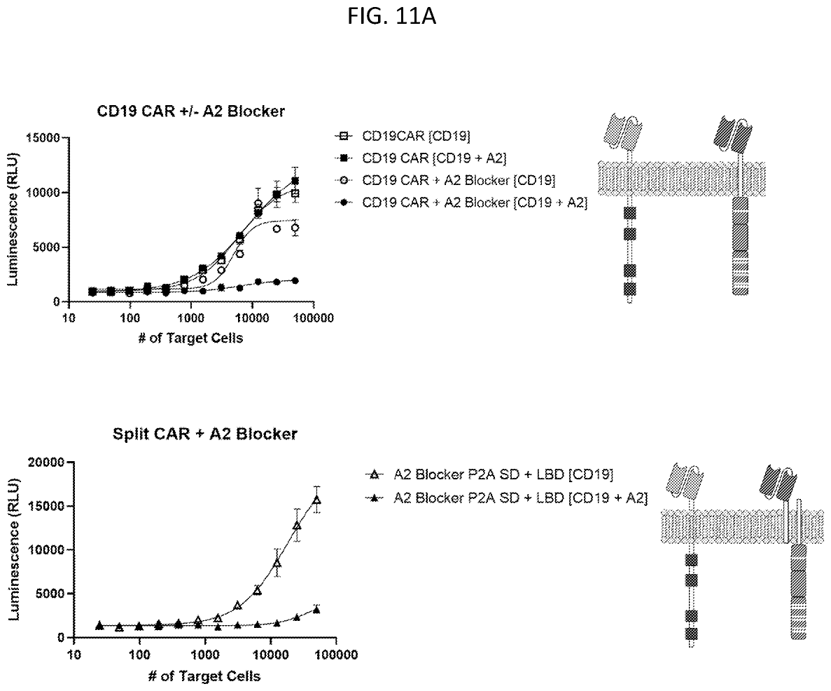

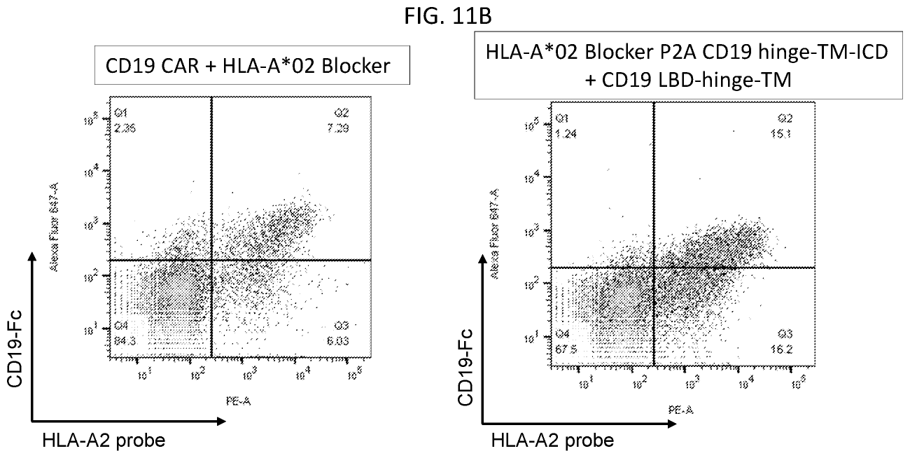

FIGS. 11A-11B show that the same approach works using a CD19 CAR paired with HLA-A*02 directed blocker. Jurkat NFAT-luciferase cells were transfected with 1 ug of activator receptor and 1 ug blocker receptor DNA using Neon transfection (1500 V, 10 ms, 3 pulses) in the case of full length receptors, and 1 ug of each vector in the case where the activator was split between two vectors (see Table X4 below). Jurkat E6.1 cells were transfected with 2 ug of either CD19 alone or 2 ug each of CD19 and A2 DNA to use as target cells. Target cell antigen expression is indicated in square brackets [ ] in FIG. 11A. Split CAR blocker mediates blocking as well as full length blocker co-expressed with full length activator. FIG. 11B shows expression of CAR, which was measured by staining with human Fc-fused CD19. Expression of the blocking receptor was measured by staining with fluorescently labeled HLA-A*02 probe.

TABLE 9
CD8a hinge constructs

| Name | DNA Sequence | Protein Sequence |
|---|---|---|
| ScFv-LIR1 | See Table 7 above | See Table 7 above |
| inhibitory | | |
| receptor | | |
| P2A | See Table 7 above | See Table 7 above |
| CD8α | ACCACGACGCCAGCGCCGCGACCACCAACACCGGCGCCC | TTTPAPRPPTPAPTIAS |
| hinge | ACCATCGCGTCGCAGCCCCTGTCCCTGCGCCCAGAGGCG | QPLSLRPEACRPAAGGA |
| | TGCCGGCCAGCGGCGGGGGGCGCAGTGCACACGAGGGGG | VHTRGLDFACD |
| | CTGGACTTCGCCTGTGAT | |
| CD8 hinge | ACCACGACGCCAGCGCCGCGACCACCAACACCGGCGCCC | TTTPAPRPPTPAPTIAS |
| (mutant | ACCATCGCGTCGCAGCCCCTGTCCCTGCGCCCAGAGGCG | QPLSLRPEASRPAAGGA |
| cysteines) | AGCCGGCCAGCGGCGGGGGGCGCAGTGCACACGAGGGGG | VHTRGLDFASD |
| | CTGGACTTCGCCAGTGAT | |
| HLA-A | GTGGGCATCATTGCTGGCCTGGTTCTCTTTGGAGCTGTG | VGIIAGLVLFGAVITGA |
| transmembrane | ATCACTGGAGCTGTGGTCGCTGCCGTGATGTGGAGGAGC | VVAAVMWRSKRSR |
| | AAGCGGAGCAGA | |
| Hinge- | GACATGAGGGTCCCCGCTCAGCTCCTGGGGCTCCTGCTA | DMRVPAQLLGLLLLWLR |
| transmembrane | CTCTGGCTCCGAGGTGCCAGATGTACCACGACGCCAGCG | GARCTTTPAPRPPTPAP |
| (wild | CCGCGACCACCAACACCGGCGCCCACCATCGCGTCGCAG | TIASQPLSLRPEACRPA |
| type CD8α | CCCCTGTCCCTGCGCCCAGAGGCGTGCCGGCCAGCGGCG | AGGAVHTRGLDFACDVG |
| underlined) | GGGGGCGCAGTGCACACGAGGGGGCTGGACTTCGCCTGT | IIAGLVLFGAVITGAVV |
| | GATGTGGGCATCATTGCTGGCCTGGTTCTCTTTGGAGCT | AAVMWRSKRSR |
| | GTGATCACTGGAGCTGTGGTCGCTGCCGTGATGTGGAGG | |
| | AGCAAGCGGAGCAGA | |
| CD28-4- | CTGCTGCACAGCGACTACATGAACATGACCCCCCGGAGG | LLHSDYMNMTPRRPGPT |
| 1BB-CD3z | CCTGGCCCCACCCGGAAGCACTACCAGCCCTACGCCCCT | RKHYQPYAPPRDFAAYR |
| | CCCAGGGATTTCGCCGCCTACCGGAGCAAACGGGGCAGA | SKRGRKKLLYIFKQPFM |
| | AAGAAACTCCTGTATATATTCAAACAACCATTTATGAGG | RPVQTTQEEDGCSCREP |
| | CCAGTACAAACTACTCAAGAGGAAGATGGCTGTAGCTGC | EEEEGGCELRVKESRSA |
| | CGATTTCCAGAAGAAGAAGAAGGAGGATGTGAACTGAGA | DAPAYKQGQNQLYNELN |
| | GTGAAGTTCAGCAGGAGCGCAGACGCCCCCGCGTACAAG | LGRREEYDVLDKRRGRD |
| | CAGGGCCAGAACCAGCTCTATAACGAGCTCAATCTAGGA | PEMGGKPRRKNPQEGLY |
| | CGAAGAGAGGAGTACGATGTTTTGGACAAGCGTAGAGGC | NELQKDKMAEAYSEIGM |
| | CGGGACCCTGAGATGGGGGGAAAGCCGAGAAGGAAGAAC | KGERRRGKGHDGLYQGL |
| | CCTCAGGAAGGCCTGTACAATGAACTGCAGAAAGATAAG | STATKDTYDALHMQALP |
| | ATGGCGGAGGCCTACAGTGAGATTGGGATGAAAGGCGAG | PR |
| | CGCCGGAGGGGCAAGGGGCACGATGGCCTTTACCAGGGA | |
| | CTCAGTACAGCCACCAAGGACACCTACGACGCCCTTCAC | |
| | ATGCAGGCCCTGCCCCCTCGC | |
| HLA-A*02 | ATGGACATGAGGGTCCCCGCTCAGCTCCTGGGGCTCCTG | MDMRVPAQLLGLLLLWL |
| SCFV LIR1 | CTACTCTGGCTCCGAGGTGCCAGATGTGATGTTTTGATG | RGARCDVLMTQTPLSLP |
| receptor; | ACCCAAACTCCACTCTCCCTGCCTGTCAGTCTTGGAGAT | VSLGDQASISCRSSQSI |
| P2A; CD8α | CAAGCCTCCATCTCTTGCAGATCTAGTCAGAGCATTGTA | VHSNGNTYLEWYLQKPG |
| hinge, | CATAGTAATGGAAACACCTATTTAGAATGGTACCTGCAG | QSPKLLIYKVSNRFSGV |
| HLA-A*02 | AAACCAGGCCAGTCTCCAAAGCTCCTGATCTACAAAGTT | PDRFSGSGSGTDFTLKI |
| TM, CD28, | TCCAACCGATTTTCTGGGGTCCCAGACAGGTTCAGTGGC | SRVEAEDLGVYYCFQGS |
| 4-1BB and | AGTGGATCAGGGACAGATTTCACACTCAAGATCAGTAGA | HVPRTSGGGTKLEIKGG |
| CD3z ICD | GTGGAGGCTGAGGATCTGGGAGTTTATTACTGCTTTCAA | GGSGGGGSGGGGSGGQV |
| | GGTTCACATGTTCCTCGGACGTCCGGTGGAGGCACCAAG | QLQQSGPELVKPGASVR |
| | CTGGAAATCAAAGGCGGAGGTGGAAGCGGAGGGGGAGGA | ISCKASGYTFTSYHIHW |
| | TCTGGCGGCGGAGGAAGCGGAGGCCAGGTCCAGCTGCAG | VKQRPGQGLEWIGWIYP |
| | CAGTCTGGACCTGAGCTGGTGAAGCCTGGGGCTTCAGTG | GNVNTEYNEKFKGKATL |
| | AGGATATCCTGCAAGGCTTCTGGCTACACCTTCACAAGT | TADKSSSTAYMHLSSLT |
| | TACCATATACATTGGGTGAAGCAGAGGCCTGGACAGGGA | SEDSAVYFCAREEITYA |
| | CTTGAGTGGATTGGATGGATTTATCCTGGAAATGTTAAT | MDYWGQGTSVTVSSYGS |
| | ACTGAGTACAATGAGAAGTTCAAGGGCAAGGCCACACTG | QSSKPYLLTHPSDPLEL |
| | ACTGCAGACAAATCGTCCAGCACAGCCTACATGCACCTC | VVSGPSGGPSSPTTGPT |
| | AGCAGCCTGACCTCTGAGGACTCTGCGGTCTATTTCTGT | STSGPEDQPLTPTGSDP |
| | GCCAGAGAGGAGATTACCTATGCTATGGACTACTGGGGT | QSGLGRHLGVVIGILVA |
| | CAAGGAACCTCAGTCACCGTGTCCTCATACGGCTCACAG | VILLLLLLLLLFLILRH |
| | AGCTCCAAACCCTACCTGCTGACTCACCCCAGTGACCCC | RRQGKHWTSTQRKADFQ |
| | CTGGAGCTCGTGGTCTCAGGACCGTCTGGGGGCCCCAGC | HPAGAVGPEPTDRGLQW |
| | TCCCCGACAACAGGCCCCACCTCCACATCTGGCCCTGAG | RSSPAADAQEENLYAAV |
| | GACCAGCCCCTCACCCCCACCGGGTCGGATCCCCAGAGT | KHTQPEDGVEMDTRSPH |
| | GGTCTGGGAAGGCACCTGGGGGTTGTGATCGGCATCTTG | DEDPQAVTYAEVKHSRP |
| | GTGGCCGTCATCCTACTGCTCCTCCTCCTCCTCCTCCTC | RREMASPPSPLSGEFLD |
| | TTCCTCATCCTCCGACATCGACGTCAGGGCAAACACTGG | TKDRQAEEDRQMDTEAA |
| | ACATCGACCCAGAGAAAGGCTGATTTCCAACATCCTGCA | ASEAPQDVTYAQLHSLT |
| | GGGGCTGTGGGGCCAGAGCCCACAGACAGAGGCCTGCAG | LRREATEPPPSQEGPSP |
| | TGGAGGTCCAGCCCAGCTGCCGATGCCCAGGAAGAAAAC | AVPSIYATLAIHGSGAT |
| | CTCTATGCTGCCGTGAAGCACACACAGCCTGAGGATGGG | NFSLLKQAGDVEENPGP |
| | GTGGAGATGGACACTCGGAGCCCACACGATGAAGACCCC | MDMRVPAQLLGLLLLWL |
| | CAGGCAGTGACGTATGCCGAGGTGAAACACTCCAGACCT | RGARCTTTPAPRPPTPA |
| | AGGAGAGAAATGGCCTCTCCTCCTTCCCCACTGTCTGGG | PTIASQPLSLRPEACRP |
| | GAATTCCTGGACACAAAGGACAGACAGGCGGAAGAGGAC | AAGGAVHTRGLDFACDV |
| | AGGCAGATGGACACTGAGGCTGCTGCATCTGAAGCCCCC | GIIAGLVLFGAVITGAV |
| | CAGGATGTGACCTACGCCCAGCTGCACAGCTTGACCCTC | VAAVMWRSKRSRLLHSD |
| | AGACGGGAGGCAACTGAGCCTCCTCCATCCCAGGAAGGG | YMNMTPRRPGPTRKHYQ |
| | CCCTCTCCAGCTGTGCCCAGCATCTACGCCACTCTGGCC | PYAPPRDFAAYRSKRGR |
| | ATCCACGGATCCGGAGCCACCAACTTCAGCCTGCTGAAG | KKLLYIFKQPFMRPVQT |
| | CAGGCCGGCGACGTGGAGGAGAACCCCGGCCCCATGGAC | TQEEDGCSCRFPEEEEG |
| | ATGAGGGTCCCCGCTCAGCTCCTGGGGCTCCTGCTACTC | GCELRVKFSRSADAPAY |
| | TGGCTCCGAGGTGCCAGATGTACCACGACGCCAGCGCCG | KQGQNQLYNELNLGRRE |
| | CGACCACCAACACCGGCGCCCACCATCGCGTCGCAGCCC | EYDVLDKRRGRDPEMGG |
| | CTGTCCCTGCGCCCAGAGGCGTGCCGGCCAGCGGCGGGG | KPRRKNPQEGLYNELQK |
| | GGCGCAGTGCACACGAGGGGGCTGGACTTCGCCTGTGAT | DKMAEAYSEIGMKGERR |
| | GTGGGCATCATTGCTGGCCTGGTTCTCTTTGGAGCTGTG | RGKGHDGLYQGLSTATK |
| | ATCACTGGAGCTGTGGTCGCTGCCGTGATGTGGAGGAGC | DTYDALHMQALPPR |
| | AAGCGGAGCAGACTGCTGCACAGCGACTACATGAACATG | |
| | ACCCCCCGGAGGCCTGGCCCCACCCGGAAGCACTACCAG | |
| | CCCTACGCCCCTCCCAGGGATTTCGCCGCCTACCGGAGC | |
| | AAACGGGGCAGAAAGAAACTCCTGTATATATTCAAACAA | |
| | CCATTTATGAGGCCAGTACAAACTACTCAAGAGGAAGAT | |
| | GGCTGTAGCTGCCGATTTCCAGAAGAAGAAGAAGGAGGA | |
| | TGTGAACTGAGAGTGAAGTTCAGCAGGAGCGCAGACGCC | |
| | CCCGCGTACAAGCAGGGCCAGAACCAGCTCTATAACGAG | |
| | CTCAATCTAGGACGAAGAGAGGAGTACGATGTTTTGGAC | |
| | AAGCGTAGAGGCCGGGACCCTGAGATGGGGGGAAAGCCG | |
| | AGAAGGAAGAACCCTCAGGAAGGCCTGTACAATGAACTG | |
| | CAGAAAGATAAGATGGCGGAGGCCTACAGTGAGATTGGG | |
| | ATGAAAGGCGAGCGCCGGAGGGGCAAGGGGCACGATGGC | |
| | CTTTACCAGGGACTCAGTACAGCCACCAAGGACACCTAC | |
| | GACGCCCTTCACATGCAGGCCCTGCCCCCTCGC | |
| HLA-A*02 | ATGGACATGAGGGTCCCCGCTCAGCTCCTGGGGCTCCTG | MDMRVPAQLLGLLLLWL |
| SCFv LIR1 | CTACTCTGGCTCCGAGGTGCCAGATGTGATGTTTTGATG | RGARCDVLMTQTPLSLP |
| receptor; | ACCCAAACTCCACTCTCCCTGCCTGTCAGTCTTGGAGAT | VSLGDQASISCRSSQSI |
| P2A; CD8α | CAAGCCTCCATCTCTTGCAGATCTAGTCAGAGCATTGTA | VHSNGNTYLEWYLQKPG |
| hinge | CATAGTAATGGAAACACCTATTTAGAATGGTACCTGCAG | QSPKLLIYKVSNRFSGV |
| (cysteines | AAACCAGGCCAGTCTCCAAAGCTCCTGATCTACAAAGTT | PDRFSGSGSGTDFTLKI |
| mutated to | TCCAACCGATTTTCTGGGGTCCCAGACAGGTTCAGTGGC | SRVEAEDLGVYYCFQGS |
| serines), | AGTGGATCAGGGACAGATTTCACACTCAAGATCAGTAGA | HVPRTSGGGTKLEIKGG |
| HLA-A*02 | GTGGAGGCTGAGGATCTGGGAGTTTATTACTGCTTTCAA | GGSGGGGSGGGGSGGQV |
| TM, CD28, | GGTTCACATGTTCCTCGGACGTCCGGTGGAGGCACCAAG | QLQQSGPELVKPGASVR |
| 4-1BB and | CTGGAAATCAAAGGCGGAGGTGGAAGCGGAGGGGGAGGA | ISCKASGYTFTSYHIHW |
| CD3z ICD | TCTGGCGGCGGAGGAAGCGGAGGCCAGGTCCAGCTGCAG | VKQRPGQGLEWIGWIYP |
| | CAGTCTGGACCTGAGCTGGTGAAGCCTGGGGCTTCAGTG | GNVNTEYNEKFKGKATL |
| | AGGATATCCTGCAAGGCTTCTGGCTACACCTTCACAAGT | TADKSSSTAYMHLSSLT |
| | TACCATATACATTGGGTGAAGCAGAGGCCTGGACAGGGA | SEDSAVYFCAREEITYA |
| | CTTGAGTGGATTGGATGGATTTATCCTGGAAATGTTAAT | MDYWGQGTSVTVSSYGS |
| | ACTGAGTACAATGAGAAGTTCAAGGGCAAGGCCACACTG | QSSKPYLLTHPSDPLEL |
| | ACTGCAGACAAATCGTCCAGCACAGCCTACATGCACCTC | VVSGPSGGPSSPTTGPT |
| | AGCAGCCTGACCTCTGAGGACTCTGCGGTCTATTTCTGT | STSGPEDQPLTPTGSDP |
| | GCCAGAGAGGAGATTACCTATGCTATGGACTACTGGGGT | QSGLGRHLGVVIGILVA |
| | CAAGGAACCTCAGTCACCGTGTCCTCATACGGCTCACAG | VILLLLLLLLLFLILRH |
| | AGCTCCAAACCCTACCTGCTGACTCACCCCAGTGACCCC | RRQGKHWTSTQRKADFQ |
| | CTGGAGCTCGTGGTCTCAGGACCGTCTGGGGGCCCCAGC | HPAGAVGPEPTDRGLQW |
| | TCCCCGACAACAGGCCCCACCTCCACATCTGGCCCTGAG | RSSPAADAQEENLYAAV |
| | GACCAGCCCCTCACCCCCACCGGGTCGGATCCCCAGAGT | KHTQPEDGVEMDTRSPH |
| | GGTCTGGGAAGGCACCTGGGGGTTGTGATCGGCATCTTG | DEDPQAVTYAEVKHSRP |
| | GTGGCCGTCATCCTACTGCTCCTCCTCCTCCTCCTCCTC | RREMASPPSPLSGEFLD |
| | TTCCTCATCCTCCGACATCGACGTCAGGGCAAACACTGG | TKDRQAEEDRQMDTEAA |
| | ACATCGACCCAGAGAAAGGCTGATTTCCAACATCCTGCA | ASEAPQDVTYAQLHSLT |
| | GGGGCTGTGGGGCCAGAGCCCACAGACAGAGGCCTGCAG | LRREATEPPPSQEGPSP |
| | TGGAGGTCCAGCCCAGCTGCCGATGCCCAGGAAGAAAAC | AVPSIYATLAIHGSGAT |
| | CTCTATGCTGCCGTGAAGCACACACAGCCTGAGGATGGG | NFSLLKQAGDVEENPGP |
| | GTGGAGATGGACACTCGGAGCCCACACGATGAAGACCCC | MDMRVPAQLLGLLLLWL |
| | CAGGCAGTGACGTATGCCGAGGTGAAACACTCCAGACCT | RGARCTTTPAPRPPTPA |
| | AGGAGAGAAATGGCCTCTCCTCCTTCCCCACTGTCTGGG | PTIASQPLSLRPEASRP |
| | GAATTCCTGGACACAAAGGACAGACAGGCGGAAGAGGAC | AAGGAVHTRGLDFASDV |
| | AGGCAGATGGACACTGAGGCTGCTGCATCTGAAGCCCCC | GIIAGLVLFGAVITGAV |
| | CAGGATGTGACCTACGCCCAGCTGCACAGCTTGACCCTC | VAAVMWRSKRSRLLHSD |
| | AGACGGGAGGCAACTGAGCCTCCTCCATCCCAGGAAGGG | YMNMTPRRPGPTRKHYQ |
| | CCCTCTCCAGCTGTGCCCAGCATCTACGCCACTCTGGCC | PYAPPRDFAAYRSKRGR |
| | ATCCACGGATCCGGAGCCACCAACTTCAGCCTGCTGAAG | KKLLYIFKQPFMRPVQT |
| | CAGGCCGGCGACGTGGAGGAGAACCCCGGCCCCATGGAC | TQEEDGCSCRFPEEEEG |
| | ATGAGGGTCCCCGCTCAGCTCCTGGGGCTCCTGCTACTC | GCELRVKESRSADAPAY |
| | TGGCTCCGAGGTGCCAGATGTACCACGACGCCAGCGCCG | KQGQNQLYNELNLGRRE |
| | CGACCACCAACACCGGCGCCCACCATCGCGTCGCAGCCC | EYDVLDKRRGRDPEMGG |
| | CTGTCCCTGCGCCCAGAGGCGAGCCGGCCAGCGGCGGGG | KPRRKNPQEGLYNELQK |
| | GGCGCAGTGCACACGAGGGGGCTGGACTTCGCCAGTGAT | DKMAEAYSEIGMKGERR |
| | GTGGGCATCATTGCTGGCCTGGTTCTCTTTGGAGCTGTG | RGKGHDGLYQGLSTATK |
| | ATCACTGGAGCTGTGGTCGCTGCCGTGATGTGGAGGAGC | DTYDALHMQALPPR |
| | AAGCGGAGCAGACTGCTGCACAGCGACTACATGAACATG | |
| | ACCCCCCGGAGGCCTGGCCCCACCCGGAAGCACTACCAG | |
| | CCCTACGCCCCTCCCAGGGATTTCGCCGCCTACCGGAGC | |
| | AAACGGGGCAGAAAGAAACTCCTGTATATATTCAAACAA | |
| | CCATTTATGAGGCCAGTACAAACTACTCAAGAGGAAGAT | |
| | GGCTGTAGCTGCCGATTTCCAGAAGAAGAAGAAGGAGGA | |
| | TGTGAACTGAGAGTGAAGTTCAGCAGGAGCGCAGACGCC | |
| | CCCGCGTACAAGCAGGGCCAGAACCAGCTCTATAACGAG | |
| | CTCAATCTAGGACGAAGAGAGGAGTACGATGTTTTGGAC | |
| | AAGCGTAGAGGCCGGGACCCTGAGATGGGGGGAAAGCCG | |
| | AGAAGGAAGAACCCTCAGGAAGGCCTGTACAATGAACTG | |
| | CAGAAAGATAAGATGGCGGAGGCCTACAGTGAGATTGGG | |
| | ATGAAAGGCGAGCGCCGGAGGGGCAAGGGGCACGATGGC | |
| | CTTTACCAGGGACTCAGTACAGCCACCAAGGACACCTAC | |
| | GACGCCCTTCACATGCAGGCCCTGCCCCCTCGC | |
| Anti CD19 | ATGCTTCTCCTGGTGACAAGCCTTCTGCTCTGTGAGTTA | MLLLVTSLLLCELPHPA |
| SCFV, | CCACACCCAGCATTCCTCCTGATCCCAGACATCCAGATG | FLLIPDIQMTQTTSSLS |
| CD8α | ACACAGACTACATCCTCCCTGTCTGCCTCTCTGGGAGAC | ASLGDRVTISCRASQDI |
| hinge, | AGAGTCACCATCAGTTGCAGGGCAAGTCAGGACATTAGT | SKYLNWYQQKPDGTVKL |
| HLA-A*02 | AAATATTTAAATTGGTATCAGCAGAAACCAGATGGAACT | LIYHTSRLHSGVPSRES |
| transmembrane | GTTAAACTCCTGATCTACCATACATCAAGATTACACTCA | GSGSGTDYSLTISNLEQ |
| domain | GGAGTCCCATCAAGGTTCAGTGGCAGTGGGTCTGGAACA | EDIATYFCQQGNTLPYT |
| | GATTATTCTCTCACCATTAGCAACCTGGAGCAAGAAGAT | FGGGTKLEITGGGGSGG |
| | ATTGCCACTTACTTTTGCCAACAGGGTAATACGCTTCCG | GGSGGGGSGGEVKLQES |
| | TACACGTTCGGAGGGGGGACCAAGCTGGAGATCACAGGC | GPGLVAPSQSLSVTCTV |
| | GGAGGTGGAAGCGGAGGGGGAGGATCTGGCGGCGGAGGA | SGVSLPDYGVSWIRQPP |
| | AGCGGAGGCGAGGTGAAACTGCAGGAGTCAGGACCTGGC | RKGLEWLGVIWGSETTY |
| | CTGGTGGCGCCCTCACAGAGCCTGTCCGTCACATGCACT | YNSALKSRLTIIKDNSK |
| | GTCTCAGGGGTCTCATTACCCGACTATGGTGTAAGCTGG | SQVFLKMNSLQTDDTAI |
| | ATTCGCCAGCCTCCACGAAAGGGTCTGGAGTGGCTGGGA | YYCAKHYYYGGSYAMDY |
| | GTAATATGGGGTAGTGAAACCACATACTATAATTCAGCT | WGQGTSVTVSSTTTPAP |
| | CTCAAATCCAGACTGACCATCATCAAGGACAACTCCAAG | RPPTPAPTIASQPLSLR |
| | AGCCAAGTTTTCTTAAAAATGAACAGTCTGCAAACTGAT | PEACRPAAGGAVHTRGL |
| | GACACAGCCATTTACTACTGTGCCAAACATTATTACTAC | DFACDVGIIAGLVLFGA |
| | GGTGGTAGCTATGCTATGGACTACTGGGGCCAAGGAACC | VITGAVVAAVMWRSKRS |
| | TCAGTCACCGTGTCCTCAACCACGACGCCAGCGCCGCGA | |
| | CCACCAACACCGGCGCCCACCATCGCGTCGCAGCCCCTG | |
| | TCCCTGCGCCCAGAGGCGTGCCGGCCAGCGGCGGGGGGC | |
| | GCAGTGCACACGAGGGGGCTGGACTTCGCCTGTGATGTG | |
| | GGCATCATTGCTGGCCTGGTTCTCTTTGGAGCTGTGATC | |
| | ACTGGAGCTGTGGTCGCTGCCGTGATGTGGAGGAGCAAG | |
| | CGGAGCA | |

### Example 3: Split Vector System Using a Fos/Jun Hinge

Additional hinge sequences that promoter dimerization can also be used, for example the Fos/Jun hinge sequences. Fos/Jun hinge sequences, and constructs incorporating these sequences, are shown in Table 10 below. An HLA-A transmembrane domain was used with the Fos/Jun hinges, as this transmembrane domain is known to exist as a monomer. This was to limit the source of dimerization to the hinge alone. Additional monomeric transmembrane domains could also have been used.

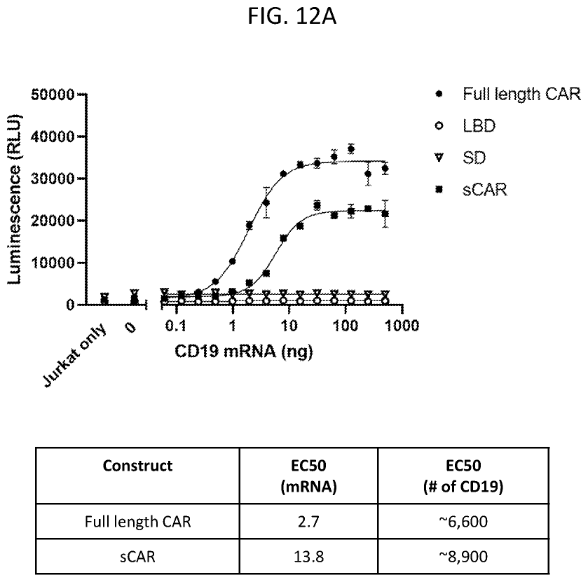

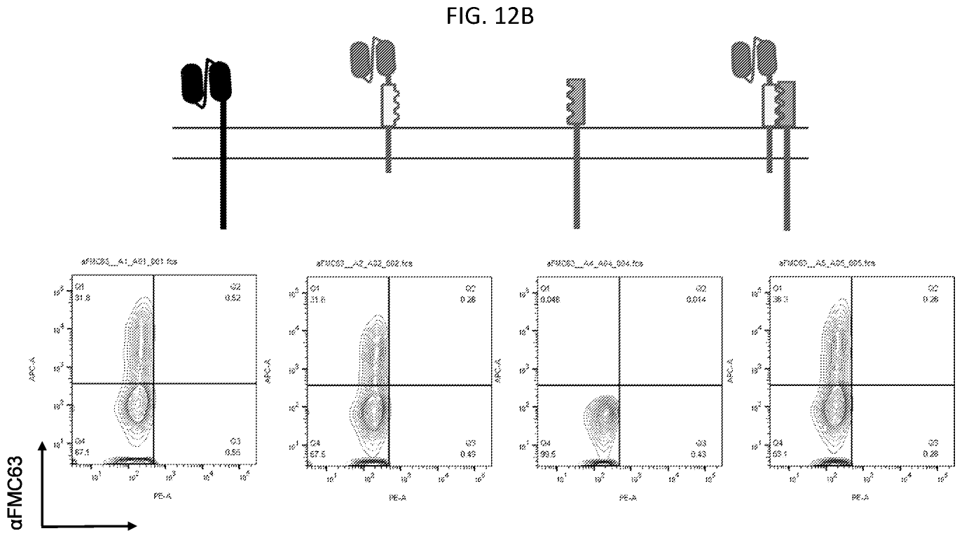

The results of using the Fos/Jun hinge are shown in FIGS. 12A-12B. For FIG. 12A, Jurkat NFAT-luciferase effector cells were transfected with 1 ug of DNA of the vectors shown in Table 10 per 1E6 cells with the Neon transfection system (1500 v, 10 ms, 3 pulses). For target cells, wild type HeLa cells were transfected with titrated CD19 mRNA using the 4D transfection system (CN114; SE buffer) and plated in 384 wells at 10000 cells per well. 18-24 hours post-transfection, Jurkat cells were harvested and resuspended in RPMI+10% heat-inactivated FBS+0.1% pen/strep at 0.66E6 cells per mL. 10000 Jurkat cells were added to HeLa cells and NFAT activity was measured using the luciferase detection system 6 hours post co-culture.

FIG. 12B shows the expression of the CD19 scFv in the Jurkat effector cells. The scFv was detected by staining with anti-FMC63 antibody.

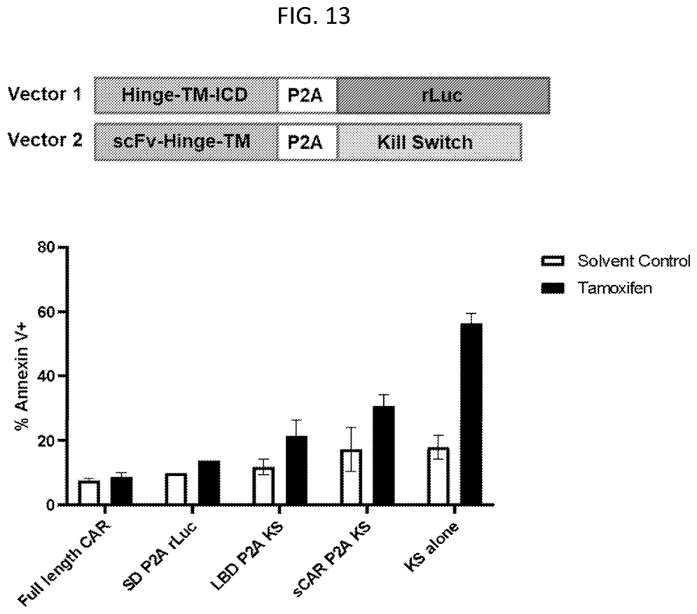

The dual vectors used in FIGS. 12A-12B also had sufficient space to incorporate a luciferase reporter and an ESR1 based kill switch (FIG. 13, top). Upon activation with tamoxifen, the kill switch gene is able to inducibly kill target cells, which was as assessed by Annexin V staining. Jurkat cell transformed with the vector that incorporated the kill switch after a P2A self-cleaving peptide showed reduced, but inducible, killing after culture with tamoxifen (FIG. 13 bottom). Jurkat cells were transfected with 1 ug each of the two vectors (FIG. 13, Table 10 below). Four hours post-transfection, 1 uM tamoxifen or the equivalent volume of ethanol was added. 18-24 hours post-transfection, cells were co-stained with Annexin V antibody (FITC) and propidium iodide. Apoptotic cells were measured using flow cytometry.

TABLE 10
Fos/Jun hinge constructs

| Name | DNA Sequence | Protein Sequence |
|---|---|---|
| Fos | AGTGATGGCAGCCTGACTGATACACTCCAAGCGGAGACAG | SDGSLTDTLQAETDQL |
| hinge | ACCAACTAGAAGATGAGAAGTCTGCTTTGCAGACCGAGAT | EDEKSALQTEIANLLK |
| | TGCCAACCTGCTGAAGGAGAAGGAAAAACTAGAGTTCATC | EKEKLEFILAAHGS |
| | CTGGCAGCTCACGGATCC | |
| Jun | AGTGATGGAAGCAGAATCGCAAGACTGGAAGAGAAGGTAA | SDGSRIARLEEKVKTL |
| hinge | AGACATTGAAGGCGCAGAATAGCGAGCTGGCTTCAACGGC | KAQNSELASTANMLRE |
| | TAATATGTTGAGGGAGCAGGTGGCTCAGTTGAAGCAGAAG | QVAQLKQKVMNHG |
| | GTGATGAACCATGGCTC | |
| CD8α | ACCACGACGCCAGCGCCGCGACCACCAACACCGGCGCCCA | TTTPAPRPPTPAPTIA |
| hinge | CCATCGCGTCGCAGCCCCTGTCCCTGCGCCCAGAGGCGAG | SQPLSLRPEASRPAAG |
| (mutant | CCGGCCAGCGGCGGGGGGCGCAGTGCACACGAGGGGGCTG | GAVHTRGLDFA |
| cysteines) | GACTTCGCC | |
| CD8α- | ACCACGACGCCAGCGCCGCGACCACCAACACCGGCGCCCA | TTTPAPRPPTPAPTIA |
| Fos | CCATCGCGTCGCAGCCCCTGTCCCTGCGCCCAGAGGCGAG | SQPLSLRPEASRPAAG |
| (Fos is | CCGGCCAGCGGCGGGGGGCGCAGTGCACACGAGGGGGCTG | GAVHTRGLDFASDGSL |
| underlined) | GACTTCGCCAGTGATGGCAGCCTGACTGATACACTCCAAG | TDTLQAETDQLEDEKS |
| | CGGAGACAGACCAACTAGAAGATGAGAAGTCTGCTTTGCA | ALQTEIANLLKEKEKL |
| | GACCGAGATTGCCAACCTGCTGAAGGAGAAGGAAAAACTA | EFILAAHGS |
| | GAGTTCATCCTGGCAGCTCACGGATCC | |
| CD8α- | ACCACGACGCCAGCGCCGCGACCACCAACACCGGCGCCCA | TTTPAPRPPTPAPTIA |
| Jun | CCATCGCGTCGCAGCCCCTGTCCCTGCGCCCAGAGGCGAG | SQPLSLRPEASRPAAG |
| (Jun is | CCGGCCAGCGGGGGGGGCGCAGTGCACACGAGGGGGCTG | GAVHTRGLDFASDGSR |
| underlined) | GACTTCGCCAGTGATGGAAGCAGAATCGCAAGACTGGAAG | IARLEEKVKTLKAQNS |
| | AGAAGGTAAAGACATTGAAGGCGCAGAATAGCGAGCTGGC | ELASTANMLREQVAQL |
| | TTCAACGGCTAATATGTTGAGGGAGCAGGTGGCTCAGTTG | KQKVMNHG |
| | AAGCAGAAGGTGATGAACCATGGCTC | |
| Alternative | AGTCGGCATCATTGCTGGCCTGGTTCTCTTTGGAGCTGTG | SRHHCWPGSLWSCDHW |
| HLA-A DNA | ATCACTGGAGCTGTGGTCGCTGCCGTGATGTGGAGGAGCA | SCGRCRDVEEQA |
| sequence | AGCGG | |
| Anti- | ATGCTTCTCCTGGTGACAAGCCTTCTGCTCTGTGAGTTAC | MLLLVTSLLLCELPHP |
| CD19 | CACACCCAGCATTCCTCCTGATCCCAGACATCCAGATGAC | AFLLIPDIQMTQTTSS |
| SCFV, | ACAGACTACATCCTCCCTGTCTGCCTCTCTGGGAGACAGA | LSASLGDRVTISCRAS |
| CD8α | GTCACCATCAGTTGCAGGGCAAGTCAGGACATTAGTAAAT | QDISKYLNWYQQKPDG |
| Fos | ATTTAAATTGGTATCAGCAGAAACCAGATGGAACTGTTAA | TVKLLIYHTSRLHSGV |
| hinge, | ACTCCTGATCTACCATACATCAAGATTACACTCAGGAGTC | PSRFSGSGSGTDYSLT |
| HLA- | CCATCAAGGTTCAGTGGCAGTGGGTCTGGAACAGATTATT | ISNLEQEDIATYFCQQ |
| A*02 | CTCTCACCATTAGCAACCTGGAGCAAGAAGATATTGCCAC | GNTLPYTFGGGTKLEI |
| transmembrane; | TTACTTTTGCCAACAGGGTAATACGCTTCCGTACACGTTC | TGGGGSGGGGSGGGGS |
| P2A, | GGAGGGGGGACCAAGCTGGAGATCACAGGCGGAGGTGGAA | GGEVKLQESGPGLVAP |
| ESR1- | GCGGAGGGGGAGGATCTGGCGGCGGAGGAAGCGGAGGCGA | SQSLSVTCTVSGVSLP |
| based | GGTGAAACTGCAGGAGTCAGGACCTGGCCTGGTGGCGCCC | DYGVSWIRQPPRKGLE |
| kill | TCACAGAGCCTGTCCGTCACATGCACTGTCTCAGGGGTCT | WLGVIWGSETTYYNSA |
| switch | CATTACCCGACTATGGTGTAAGCTGGATTCGCCAGCCTCC | LKSRLTIIKDNSKSQV |
| | ACGAAAGGGTCTGGAGTGGCTGGGAGTAATATGGGGTAGT | FLKMNSLQTDDTAIYY |
| | GAAACCACATACTATAATTCAGCTCTCAAATCCAGACTGA | CAKHYYYGGSYAMDYW |
| | CCATCATCAAGGACAACTCCAAGAGCCAAGTTTTCTTAAA | GQGTSVTVSSTTTPAP |
| | AATGAACAGTCTGCAAACTGATGACACAGCCATTTACTAC | RPPTPAPTIASQPLSL |
| | TGTGCCAAACATTATTACTACGGTGGTAGCTATGCTATGG | RPEASRPAAGGAVHTR |
| | ACTACTGGGGCCAAGGAACCTCAGTCACCGTGTCCTCAAC | GLDFASDGSLTDTLQA |
| | CACGACGCCAGCGCCGCGACCACCAACACCGGCGCCCACC | ETDQLEDEKSALQTEI |
| | ATCGCGTCGCAGCCCCTGTCCCTGCGCCCAGAGGCGAGCC | ANLLKEKEKLEFILAA |
| | GGCCAGCGGCGGGGGGCGCAGTGCACACGAGGGGGCTGGA | HGSVGIIAGLVLFGAV |
| | CTTCGCCAGTGATGGCAGCCTGACTGATACACTCCAAGCG | ITGAVVAAVMWRSKRS |
| | GAGACAGACCAACTAGAAGATGAGAAGTCTGCTTTGCAGA | RGSGATNFSLLKQAGD |
| | CCGAGATTGCCAACCTGCTGAAGGAGAAGGAAAAACTAGA | VEENPGPMSQEDIIRN |
| | GTTCATCCTGGCAGCTCACGGATCCGTGGGCATCATTGCT | IARHLAQVGDSMDRSI |
| | GGCCTGGTTCTCTTTGGAGCTGTGATCACTGGAGCTGTGG | PPGEKTMLVLALLLAK |
| | TCGCTGCCGTGATGTGGAGGAGCAAGCGGAGCAGAGGATC | KVASHTPSLLRDVFHT |
| | CGGAGCCACCAACTTCAGCCTGCTGAAGCAGGCCGGCGAC | TVNFINQNLGSKKNSL |
| | GTGGAGGAGAACCCCGGCCCCATGAGTCAAGAAGACATCA | ALSLTADQMVSALLDA |
| | TCCGGAATATTGCCAGGCACCTCGCCCAGGTCGGGGACAG | EPPILYSEYDPTRPES |
| | CATGGACCGTAGCATCCCTCCGGGCGAGAAGACCATGCTG | EASMMGLLTNLADREL |
| | GTGCTGGCCCTGCTGCTGGCCAAGAAGGTGGCCAGTCACA | VHMINWAKRVPGFVDL |
| | CGCCGTCCTTGCTCCGTGATGTCTTTCACACAACAGTGAA | TLHDQVHLLECAWLEI |
| | TTTTATTAACCAGAACCTAGGAAGCAAGAAGAACAGCCTG | LMIGLVWRSMEHPGKL |
| | GCCTTGTCCCTGACGGCCGACCAGATGGTCAGTGCCTTGT | LFAPNLLLDRNQGKCV |
| | TGGATGCTGAGCCCCCCATACTCTATTCCGAGTATGATCC | EGMVEIFDMLLATSSR |
| | TACCAGACCCTTCAGTGAAGCTTCGATGATGGGCTTACTG | FRMMNLQGEEFVCLKS |
| | ACCAACCTGGCAGACAGGGAGCTGGTTCACATGATCAACT | IILLNSGVYTELSSTL |
| | GGGCGAAGAGGGTGCCAGGCTTTGTGGATTTGACCCTCCA | KSLEEKDHIHRVLDKI |
| | TGATCAGGTCCACCTTCTAGAATGTGCCTGGCTAGAGATC | TDTLIHLMAKAGLTLQ |
| | CTGATGATTGGACTCGTCTGGCGCTCCATGGAGCACCCAG | QQHQRLAQLLLILSHI |
| | GGAAGCTACTGTTTGCTCCTAACTTGCTCTTGGACAGGAA | RHMSNKGMEHLYSMKC |
| | CCAGGGAAAATGTGTAGAGGGCATGGTGGAGATCTTCGAC | KNVVPLYDLLLEMLDA |
| | ATGCTGCTGGCTACATCATCTCGGTTCCGCATGATGAATC | HRLHAPT |
| | TGCAGGGAGAGGAGTTTGTGTGCCTCAAATCTATTATTTT | |
| | GCTTAATTCTGGAGTGTACACATTTCTGTCCAGCACCCTG | |
| | AAGTCTCTGGAAGAGAAGGACCATATCCACCGAGTCCTGG | |
| | ACAAGATCACAGACACTTTGATCCACCTGATGGCCAAGGC | |
| | AGGCCTGACCCTGCAGCAGCAGCACCAGCGGCTGGCCCAG | |
| | CTCCTCCTCATCCTCTCCCACATCAGGCACATGAGTAACA | |
| | AAGGCATGGAGCATCTGTACAGCATGAAGTGCAAGAACGT | |
| | GGTGCCCCTCTATGACCTGCTGCTGGAGATGCTGGACGCC | |
| | CACCGCCTACATGCGCCCACC | |
| Vk1, | ATGGACATGAGGGTCCCCGCTCAGCTCCTGGGGCTCCTGC | MDMRVPAQLLGLLLLW |
| CD8α | TACTCTGGCTCCGAGGTGCCAGATGTACCACGACGCCAGC | LRGARCTTTPAPRPPT |
| (mutated | GCCGCGACCACCAACACCGGCGCCCACCATCGCGTCGCAG | PAPTIASQPLSLRPEA |
| cysteines) | CCCCTGTCCCTGCGCCCAGAGGCGAGCCGGCCAGCGGCGG | SRPAAGGAVHTRGLDF |
| Jun | GGGGCGCAGTGCACACGAGGGGGCTGGACTTCGCCAGTGA | ASDGSRIARLEEKVKT |
| hinge, | TGGAAGCAGAATCGCAAGACTGGAAGAGAAGGTAAAGACA | LKAQNSELASTANMLR |
| HLA- | TTGAAGGCGCAGAATAGCGAGCTGGCTTCAACGGCTAATA | EQVAQLKQKVMNHGSV |
| A*02 | TGTTGAGGGAGCAGGTGGCTCAGTTGAAGCAGAAGGTGAT | GIIAGLVLFGAVITGA |
| transmembrane, | GAACCATGGCTCAGTCGGCATCATTGCTGGCCTGGTTCTC | VVAAVMWRSKRSRLLH |
| CD28, | TTTGGAGCTGTGATCACTGGAGCTGTGGTCGCTGCCGTGA | SDYMNMTPRRPGPTRK |
| 4-1BB | TGTGGAGGAGCAAGCGGAGCAGACTGCTGCACAGCGACTA | HYQPYAPPRDFAAYRS |
| and | CATGAACATGACCCCCCGGAGGCCTGGCCCCACCCGGAAG | KRGRKKLLYIFKQPFM |
| CD3z | CACTACCAGCCCTACGCCCCTCCCAGGGATTTCGCCGCCT | RPVQTTQEEDGCSCRF |
| ICD; | ACCGGAGCAAACGGGGCAGAAAGAAACTCCTGTATATATT | PEEEEGGCELRVKESR |
| P2A; | CAAACAACCATTTATGAGGCCAGTACAAACTACTCAAGAG | SADAPAYKQGQNQLYN |
| Renilla | GAAGATGGCTGTAGCTGCCGATTTCCAGAAGAAGAAGAAG | ELNLGRREEYDVLDKR |
| Luciferase | GAGGATGTGAACTGAGAGTGAAGTTCAGCAGGAGCGCAGA | RGRDPEMGGKPRRKNP |
| | CGCCCCCGCGTACAAGCAGGGCCAGAACCAGCTCTATAAC | QEGLYNELQKDKMAEA |
| | GAGCTCAATCTAGGACGAAGAGAGGAGTACGATGTTTTGG | YSEIGMKGERRRGKGH |
| | ACAAGCGTAGAGGCCGGGACCCTGAGATGGGGGGAAAGCC | DGLYQGLSTATKDTYD |
| | GAGAAGGAAGAACCCTCAGGAAGGCCTGTACAATGAACTG | ALHMQALPPRGSGATN |
| | CAGAAAGATAAGATGGCGGAGGCCTACAGTGAGATTGGGA | FSLLKQAGDVEENPGP |
| | TGAAAGGCGAGCGCCGGAGGGGCAAGGGGCACGATGGCCT | MTSKVYDPEQRKRMIT |
| | TTACCAGGGACTCAGTACAGCCACCAAGGACACCTACGAC | GPQWWARCKQMNVLDS |
| | GCCCTTCACATGCAGGCCCTGCCCCCTCGCGGATCCGGAG | FINYYDSEKHAENAVI |
| | CCACCAACTTCAGCCTGCTGAAGCAGGCCGGCGACGTGGA | FLHGNAASSYLWRHVV |
| | GGAGAACCCCGGCCCCATGACCAGCAAGGTGTACGATCCC | PHIEPVARCIIPDLIG |
| | GAGCAGAGGAAGCGCATGATCACAGGCCCTCAGTGGTGGG | MGKSGKSGNGSYRLLD |
| | CCAGGTGCAAGCAGATGAACGTGCTGGATAGCTTTATCAA | HYKYLTAWFELLNLPK |
| | TTACTATGACTCCGAGAAGCACGCCGAGAACGCCGTGATC | KIIFVGHDWGACLAFH |
| | TTCCTGCACGGCAATGCCGCCAGCTCCTACCTGTGGAGGC | YSYEHQDKIKAIVHAE |
| | ACGTGGTGCCCCACATCGAGCCTGTGGCAAGGTGTATCAT | SVVDVIESWDEWPDIE |
| | CCCCGATCTGATCGGCATGGGCAAGTCTGGCAAGAGCGGC | EDIALIKSEEGEKMVL |
| | AACGGCTCCTATCGCCTGCTGGACCACTACAAGTATCTGA | ENNFFVETMLPSKIMR |
| | CCGCCTGGTTCGAGCTGCTGAATCTGCCTAAGAAGATCAT | KLEPEEFAAYLEPFKE |
| | CTTTGTGGGACACGACTGGGGAGCATGCCTGGCATTCCAC | KGEVRRPTLSWPREIP |
| | TACTCTTATGAGCACCAGGATAAGATCAAGGCCATCGTGC | LVKGGKPDVVQIVRNY |
| | ACGCCGAGTCTGTGGTGGACGTGATCGAGAGCTGGGACGA | NAYLRASDDLPKMFIE |
| | GTGGCCCGACATCGAGGAGGACATCGCCCTGATCAAGAGC | SDPGFFSNAIVEGAKK |
| | GAGGAGGGCGAGAAGATGGTGCTGGAGAACAATTTCTTTG | FPNTEFVKVKGLHESQ |
| | TGGAGACCATGCTGCCCTCCAAGATCATGCGGAAGCTGGA | EDAPDEMGKYIKSFVE |
| | GCCTGAGGAGTTCGCCGCCTACCTGGAGCCCTTCAAGGAG | RVLKNEQ |
| | AAGGGCGAGGTGCGGAGACCAACACTGTCTTGGCCAAGAG | |
| | AGATCCCCCTGGTGAAGGGCGGCAAGCCCGATGTGGTGCA | |
| | GATCGTGCGGAACTACAATGCCTATCTGAGAGCCAGCGAC | |
| | GATCTGCCTAAGATGTTTATCGAGTCCGACCCAGGCTTCT | |
| | TTTCTAACGCCATCGTGGAGGGCGCCAAGAAGTTCCCTAA | |
| | TACAGAGTTTGTGAAGGTGAAGGGCCTGCACTTTTCCCAG | |
| | GAGGACGCCCCAGATGAGATGGGCAAGTATATCAAGTCTT | |
| | TCGTGGAGCGGGTGCTGAAGAACGAGCAG | |

What is claimed is:

1. A vector system for delivery of two polynucleotides to the same cell, comprising:
a. a first vector, wherein the first vector comprises a first polynucleotide, the first polynucleotide comprising a sequence encoding an inhibitory receptor and a sequence encoding a first part of an activator receptor, wherein the activator receptor comprises an antigen binding domain, at least a first hinge domain, at least a first transmembrane domain and intracellular domain; and
b. a second vector, wherein the second vector comprises a second polynucleotide, the second polynucleotide comprising a sequence encoding a second part of the activator receptor,
whereby the first and second parts of the activator receptor together form a functional activator receptor when co-expressed by a cell.

2. The vector system of claim 1, wherein the first polynucleotide comprises a sequence encoding a self-cleaving polypeptide or an internal ribosome entry site (IRES) between the sequence encoding the inhibitory receptor and the sequence encoding the first part of the activator.

3. The vector of claim 1, wherein the first polynucleotide comprises a sequence encoding a promoter between the sequence encoding the inhibitory receptor and the sequence encoding the first part of the activator.

4. The vector of claim 3, wherein the promoter is operably linked to the sequence encoding the first part of the activator.

5. The vector system of claim 2, wherein the self-cleaving polypeptide comprises a P2A self-cleaving polypeptide.

6. The vector system of any one of claims 1-5, wherein the activator receptor is a chimeric antigen receptor (CAR).

7. The method of claim 6, wherein the antigen binding domain of the activator receptor comprises a Fab.

8. The vector system of claim 7, wherein the first part of the activator receptor comprises a light chain of the Fab, and the second part of the activator receptor comprises a heavy chain of the Fab, the transmembrane domain and the intracellular domain.

9. The vector system of claim 7, wherein the first part of the activator receptor comprises a heavy chain of the Fab, and the second part of the activator receptor comprises a light chain of the Fab, the transmembrane domain and the intracellular domain.

10. The vector system of any one of claims 6-9, wherein the transmembrane domain comprises a CD8α, CD4, CD28, CD3ζ, or immunoglobulin G (IgG) transmembrane domain.

11. The vector system of any one of claims 6-10, wherein the hinge comprises an CD8α, CD4, CD28, CD3ζ or IgG hinge.

12. The vector system of any one of claims 1-6, wherein the first part of the activator receptor comprises a first hinge domain, a first transmembrane domain and the intracellular domain, and the second part of the activator comprises the antigen binding domain, a second hinge domain and a second transmembrane domain.

13. The vector system of any one of claims 1-6, wherein the first part of the activator comprises the antigen binding domain, a first hinge domain and a first transmembrane domain, and the second part of the activator comprises a second hinge domain, a second transmembrane domain and the intracellular domain.

14. The vector system of claim 12 or 13, wherein the first and second hinge domains comprise CD8α hinge domains.

15. The vector system of claim 14, wherein the CD8α hinge domain comprises a sequence of TTTPAPRPPTPAPTIASQPLSLRPEACRPAAGGAVHTRGLDFACD, or a sequence having at least 90%, at least 95%, or at least 97% identity thereto.

16. The vector system of any one of claims 12-15, wherein association of the CD8α hinges of the first and second parts of the activator receptor in the cell produces a functional activator receptor.

17. The vector system of claim 12 or 13, wherein the first hinge domain comprises a Fos hinge domain, and the second hinge domain comprises a Jun hinge domain.

18. The vector system of claim 12 or 13, wherein the first hinge domain comprises a Jun hinge domain, and the second hinge domain comprises a Fos hinge domain.

19. The vector system of claim 17 or 18, wherein the Fos hinge domain comprises a sequence of SDGSLTDTLQAETDQLEDEKSALQTEIANLLKEKEKLEFILAAHGS, or a sequence having at least 90%, at least 95%, or at least 97% identity thereto.

20. The vector system of any one of claims 17-19, wherein the Fos hinge further comprises a CD8α hinge sequence comprising one or more substitutions of serines for cysteines.

21. The vector system of any one of claims 17-20, wherein the Fos hinge comprises a sequence of TTTPAPRPPTPAPTIASQPLSLRPEASRPAAGGAVHTRGLDFASDGSLTDTL QAETDQLEDEKSALQTEIANLLKEKEKLEFILAAHGS, or a sequence having at least 90%, at least 95%, or at least 97% identity thereto.

22. The vector system of any one of claims 17-21, wherein the Jun hinge comprises a sequence of SDGSRIARLEEKVKTLKAQNSELASTANMLREQVAQLKQKVMNHG, or a sequence having at least 90%, at least 95%, or at least 97% identity thereto.

23. The vector system of claim 19, wherein the Jun hinge further comprises a CD8α hinge sequence comprising one or more substitutions of serines for cysteines.

24. The vector system of claim 23, wherein the Jun hinge comprises a sequence of TTTPAPRPPTPAPTIASQPLSLRPEASRPAAGGAVHTRGLDFASDGSRIARLE EKVKTLKAQNSELASTANMLREQVAQLKQKVMNHG, or a sequence having at least 90%, at least 95%, or at least 97% identity thereto.

25. The vector system of any one of claims 17-24, wherein association of the Fos and Jun hinges in the cell produces a functional activator receptor.

26. The vector system of any one of claims 12-25, wherein the activator receptor comprises an HLA-A transmembrane domain.

27. The vector system of claim 26, wherein the HLA-A transmembrane comprises a sequence of VGIIAGLVLFGAVITGAVVAAVMWRSKRSR, or a sequence having at least 90%, at least 95%, or at least 97% identity thereto.

28. The vector system of any one of claims 1-27, wherein the activator receptor comprises a CD3ζ intracellular domain.

29. The vector system of any one of claims 1-28, wherein the intracellular domain of the activator receptor comprises one or more co-stimulatory domains.

30. The vector system of claim 29, wherein the one or more co-stimulatory domains comprises an isolated or derived from CD27 molecule (CD27), CD28, CD137, TNF receptor superfamily member 4 (OX40), TNF receptor superfamily member 8 (CD30), CD40 molecule (CD40), CD40 ligand (CD40L), CD3ζ, integrin subunit beta 2 (LFA-1), inducible T cell costimulator (ICOS), CD2 molecule (CD2), CD7 molecule (CD7), TNF superfamily member 14 (LIGHT), killer cell lectin like receptor C2 (NKG2C), CD276 molecule (B7-H3), or hematopoietic cell signal transducer (DAP10).

31. The vector system of any one of claims 1-5, wherein the activator receptor is a T cell receptor.

32. The vector system of claim 30, wherein the first part of the activator receptor comprises a TCRα chain, and the second part of the activator receptor comprises a TCRβ chain.

33. The vector system of claim 30, wherein the first part of the activator receptor comprises a TCRβ chain, and the second part of the activator receptor comprises a TCRα chain.

34. The vector system of any one of claims 1-33, wherein the inhibitory receptor comprises an intracellular domain isolated or derived from LILRB1.

35. The vector system of claim 34, wherein the inhibitory receptor comprises a transmembrane domain.

36. The vector system of claim 35, wherein the transmembrane domain is isolated or derived from TCRa, TCRb, CD8α, CD28 or LILRB1.

37. The vector system of claim 36, wherein the inhibitory receptor further comprises an extracellular hinge domain.

38. The vector system of claim 37, wherein the extracellular hinge domain comprises a hinge domain isolated or derived from CD8α, CD28 or LILRB1.

39. The vector system of any one of claims 34-38, wherein the hinge, transmembrane, and intracellular domains comprise a sequence at least 90%, at least 95%, at least 99%, or 100% identical to YGSQSSKPYLLTHPSDPLELVVSGPSGGPSSPTTGPTSTSGPEDQPLTPTGSD PQSGLGRHLGVVIGILVAVILLLLLLLLLFLILRHRRQGKHWTSTQRKADFQ HPAGAVGPEPTDRGLQWRSSPAADAQEENLYAAVKHTQPEDGVEMDTRS PHDEDPQAVTYAEVKHSRPRREMASPPSPLSGEFLDTKDRQAEEDRQMDT EAAASEAPQDVTYAQLHSLTLRREATEPPPSQEGPSPAVPSIYATLAIH, or a sequence having at least 90%, at least 95%, or at least 97% identity thereto.

40. The vector system of any one of claims 1-39, wherein the inhibitory receptor comprises an antigen binding domain specific to an antigen whose expression is lost in a cancer cell through loss of heterozygosity.

41. The vector system of claim 40, wherein the inhibitory receptor comprises an antigen binding domain specific to an HLA-A allele, an HLA-B allele, an HLA-C allele, an HLA-E allele, an HLA-F allele, an HLA-G allele, a minor histocompatibility antigen (MiHA), or a Y chromosome antigen.

42. The vector system of any one of claims 1-41, wherein the activator receptor comprises an antigen binding domain specific to a cancer antigen.

43. The vector system of any one of claims 1-42, wherein the first and/or second polynucleotide comprises a polynucleotide sequence encoding an additional gene.

44. The vector system of claim 43, wherein the additional gene comprises a marker, a reporter, a short interfering RNA, or an inducible kill switch.

45. The vector system of any one of claims 1-44, wherein the first and second vectors are plasmids.

46. The vector system of any one of claims 1-44, wherein the first and second vectors are viral vectors.

47. The vector system of claim 46, wherein the viral vectors are adeno-associated viral (AAV) viral vectors or retroviral vectors.

48. The vector system of claim 47, wherein the retroviral vectors are lentiviral vectors.

49. A cell, comprising the first and second vectors of any one of claims 1-48.

50. The cell of claim 49, wherein the cell expresses a functional activator receptor and a functional inhibitory receptor.

51. The cell of claim 49 or 50, wherein the cell is an immune cell.

52. The method of claim 51, wherein the immune cell is a T cell, B cell, macrophage or an NK cell.

53. A pharmaceutical composition, comprising the cell of any one of claims 49-52, and a pharmaceutically acceptable carrier, diluent, or excipient.

54. A method of treating cancer in a subject, comprising administering a therapeutically effective amount of the cell of claim 49 or 50, or the pharmaceutical composition of claim 53.

55. A method of making a recombinant immune cell, comprising:
a. providing a plurality of immune cells; and
b. transforming the plurality of immune cells with the vector system of any one of claims 1-48.


SEQUENCE LISTING (177 sequences)

SEQ ID NO: 1 - protein, 107 aa, synthetic construct [sequence omitted]

SEQ ID NO: 2 - protein, 159 aa, synthetic construct
WGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCTTTPAPRPPTPAPTIASQPLSLRPEACRPAAGGAVHTRGLDFACD

SEQ ID NO: 3 - protein, 159 aa, synthetic construct
WGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCTTTPAPRPPTPAPTIASQPLSLRPEACRPAAGGAVHTRGLDFACD

SEQ ID NO: 4 - protein, 222 aa, synthetic construct
FWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 5 - protein, 45 aa, synthetic construct
TTTPAPRPPTPAPTIASQPLSLRPEACRPAAGGAVHTRGLDFACD

SEQ ID NO: 6 - protein, 46 aa, synthetic construct
SDGSLTDTLQAETDQLEDEKSALQTEIANLLKEKEKLEFILAAHGS

SEQ ID NO: 7 - protein, 45 aa, synthetic construct
TTTPAPRPPTPAPTIASQPLSLRPEASRPAAGGAVHTRGLDFASD

SEQ ID NO: 8 - protein, 89 aa, synthetic construct
TTTPAPRPPTPAPTIASQPLSLRPEASRPAAGGAVHTRGLDFASDGSLTDTLQAETDQLEDEKSALQTEIANLLKEKEKLEFILAAHGS

SEQ ID NO: 9 - protein, 45 aa, synthetic construct
SDGSRIARLEEKVKTLKAQNSELASTANMLREQVAQLKQKVMNHG

SEQ ID NO: 10 - protein, 88 aa, synthetic construct
TTTPAPRPPTPAPTIASQPLSLRPEASRPAAGGAVHTRGLDFASDGSRIARLEEKVKTLKAQNSELASTANMLREQVAQLKQKVMNHG

SEQ ID NO: 11 - protein, 30 aa, synthetic construct
VGIIAGLVLFGAVITGAVVAAVMWRSKRSR

SEQ ID NO: 12 - protein, 189 aa, synthetic construct
LLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 13 - protein, 18 aa, synthetic construct
EGRGSLLTCGDVEENPGP

SEQ ID NO: 14 - protein, 19 aa, synthetic construct
ATNFSLLKQAGDVEENPGP

SEQ ID NO: 15 - protein, 20 aa, synthetic construct
QCTNYALLKLAGDVESNPGP

SEQ ID NO: 16 - protein, 22 aa, synthetic construct
VKQTLNFDLLKLAGDVESNPGP

SEQ ID NO: 17 - protein, 22 aa, synthetic construct
GSGATNFSLLKQAGDVEENPGP

SEQ ID NO: 18 - protein, 21 aa, synthetic construct
GSGEGRGSLLTCGDVEENPGP

SEQ ID NO: 19 - protein, 23 aa, synthetic construct
GSGQCTNYALLKLAGDVESNPGP

SEQ ID NO: 20 - protein, 25 aa, synthetic construct
GSGVKQTLNFDLLKLAGDVESNPGP

SEQ ID NO: 21 - skipped

SEQ ID NO: 22 - protein, 7 aa, synthetic construct
ENLYTQS

SEQ ID NO: 23 - protein, 5 aa, synthetic construct
DDDDK

SEQ ID NO: 24 - protein, 4 aa, synthetic construct
LVPR

SEQ ID NO: 25 - protein, 10 aa, synthetic construct
CGLVPAGSGP

SEQ ID NO: 26 - skipped

SEQ ID NO: 27 - protein, 8 aa, synthetic construct
LEVLFQGP

SEQ ID NO: 28 - protein, 12 aa, synthetic construct
SLLKSRMVPNFN

SEQ ID NO: 29 - protein, 12 aa, synthetic construct
SLLIARRMPNFN

SEQ ID NO: 30 - protein, 12 aa, synthetic construct
SKLVQASASGVN

SEQ ID NO: 31 - protein, 12 aa, synthetic construct
SSYLKASDAPDN

SEQ ID NO: 32 - protein, 12 aa, synthetic construct
RPKPQQFFGLMN

SEQ ID NO: 33 - protein, 12 aa, synthetic construct
SLRPLALWRSFN

SEQ ID NO: 34 - protein, 12 aa, synthetic construct
SPQGIAGQRNFN

SEQ ID NO: 35 - protein, 14 aa, synthetic construct
DVDERDVRGFASFL

SEQ ID NO: 36 - protein, 12 aa, synthetic construct
SLPLGLWAPNFN

SEQ ID NO: 37 - protein, 12 aa, synthetic construct
SLLIFRSWANFN

SEQ ID NO: 38 - protein, 11 aa, synthetic construct
SGWIATVIVIT

SEQ ID NO: 39 - protein, 12 aa, synthetic construct
SLGPQGIWGQFN

SEQ ID NO: 40 - protein, 11 aa, synthetic construct
KKSPGRWGGSV

SEQ ID NO: 41 - protein, 12 aa, synthetic construct
PQGLLGAPGILG

SEQ ID NO: 42 - protein, 31 aa, synthetic construct
HGPEGLRVGFYESDVMGRGHARLVHVEEPHT

SEQ ID NO: 43 - protein, 12 aa, synthetic construct
GPQGLAGQRGIV

SEQ ID NO: 44 - protein, 12 aa, synthetic construct
GGSGQRGRKALE

SEQ ID NO: 45 - protein, 12 aa, synthetic construct
SLSALLSSDIFN

SEQ ID NO: 46 - protein, 12 aa, synthetic construct
SLPRFKIIGGFN

SEQ ID NO: 47 - protein, 12 aa, synthetic construct
SLLGIAVPGNFN

SEQ ID NO: 48 - protein, 12 aa, synthetic construct
FFKNIVTPRTPP

SEQ ID NO: 49 - protein, 22 aa, synthetic construct
MDMRVPAQLLGLLLLWLRGARC

SEQ ID NO: 50 - protein, 41 aa, synthetic construct
CTIEVMYPPPYLDNEKSNGTIIHVKGKHLCPSPLFPGPSKP

SEQ ID NO: 51 - protein, 267 aa, synthetic construct
TTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 52 - protein, 200 aa, synthetic construct
RSKRSRLLHSDYMNMTPRRPGPTRKHYQPYAPPRDFAAYRSRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 53 - DNA, 603 bp, synthetic construct
  source 1..603: mol_type = other DNA
aggagtaagaggagcaggctcctgcacagtgactacatgaacatgactccccgccgccccgggcccacccgcaagcattaccagccctatgccccaccacgcgacttcgcagcctatcgctcccgtttctctgttgttaaacggggcagaaagaagctcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctag

SEQ ID NO: 54 - DNA, 666 bp, synthetic construct
  source 1..666: mol_type = other DNA
ttctgggtgctggtcgttgtgggcggcgtgctggcctgctacagcctgctggtgacagtggccttcatcatcttttgggtgaggagcaagcggagcagactgctgcacagcgactacatgaacatgaccccccggaggcctggccccacccggaagcactaccagccctacgcccctcccagggatttcgccgcctaccggagcaaacggggcagaaagaaactcctgtatatattcaaacaaccatttatgaggccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagcgtagaggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggactcagtacagccaccaaggacacctacgacgcccttcacatgcaggccctgccccctcgc

SEQ ID NO: 55 - protein, 153 aa, synthetic construct
RSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 56 - DNA, 462 bp, synthetic construct
  source 1..462: mol_type = other DNA
aggagcaagcggagcagactgctgcacagcgactacatgaacatgaccccccggaggcctggccccacccggaagcactaccagccctacgcccctcccagggatttcgccgcctaccggagcagagtgaagttcagcaggagcgcagacgcccccgcgtacaagcagggccagaaccagctctataacgagctcaatctaggacgaagagaggagtacgatgttttggacaagcgtagaggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggactcagtacagccaccaaggacacctacgacgcccttcacatgcaggccctgccccctcgctag

SEQ ID NO: 57 - protein, 157 aa, synthetic construct
KRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 58 - DNA, 474 bp, synthetic construct
  source 1..474: mol_type = other DNA
aaacggggcagaaagaagctcctgtatatattcaaacaaccatttatgagaccagtacaaactactcaagaggaagatggctgtagctgccgatttccagaagaagaagaaggaggatgtgaactgggcggtggcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctag

SEQ ID NO: 59 - protein, 269 aa, synthetic construct
MDMRVPAQLLGLLLLWLRGARCDVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPRTSGGGTKLEIKGGGGSGGGGSGGGGSGGQVQLQQSGPELVKPGASVRISCKASGYTFTSYHIHWVKQRPGQGLEWIGWIYPGNVNTEYNEKFKGKATLTADKSSSTAYMHLSSLTSEDSAVYFCAREEITYAMDYWGQGTSVTVSS

SEQ ID NO: 60 - DNA, 807 bp, synthetic construct
  source 1..807: mol_type = other DNA
atggacatgagggtccccgctcagctcctggggctcctgctactctggctccgaggtgccagatgtgatgttttgatgacccaaactccactctccctgcctgtcagtcttggagatcaagcctccatctcttgcagatctagtcagagcattgtacatagtaatggaaacacctatttagaatggtacctgcagaaaccaggccagtctccaaagctcctgatctacaaagtttccaaccgattttctggggtcccagacaggttcagtggcagtggatcagggacagatttcacactcaagatcagtagagtggaggctgaggatctgggagtttattactgctttcaaggttcacatgttcctcggacgtccggtggaggcaccaagctggaaatcaaaggcggaggtggaagcggagggggaggatctggcggcggaggaagcggaggccaggtccagctgcagcagtctggacctgagctggtgaagcctggggcttcagtgaggatatcctgcaaggcttctggctacaccttcacaagttaccatatacattgggtgaagcagaggcctggacagggacttgagtggattggatggatttatcctggaaatgttaatactgagtacaatgagaagttcaagggcaaggccacactgactgcagacaaatcgtccagcacagcctacatgcacctcagcagcctgacctctgaggactctgcggtctatttctgtgccagagaggagattacctatgctatggactactggggtcaaggaacctcagtcaccgtgtcctca

SEQ ID NO: 61 - protein, 269 aa, synthetic construct
MDMRVPAQLLGLLLLWLRGARCQVQLVQSGAEVKKPGSSVKVSCKASGYTFTSYHIHWVRQAPGQGLEWIGWIYPGNVNTEYNEKFKGKATITADESTNTAYMELSSLRSEDTAVYYCAREEITYAMDYWGQGTLVTVSSGGGGSGGGGSGGGGSGGDIQMTQSPSTLSASVGDRVTITCRSSQSIVHSNGNTYLEWYQQKPGKAPKLLIYKVSNRFSGVPARFSGSGSGTEFTLTISSLQPDDFATYYCFQGSHVPRTFGQGTKVEVK

SEQ ID NO: 62 - DNA, 807 bp, synthetic construct
  source 1..807: mol_type = other DNA
atggacatgagggtccccgctcagctcctggggctcctgctactctggctccgaggtgccagatgtcaggtgcagctggtgcagtctggggctgaggtgaagaagcctgggtcctcagtgaaggtttcctgcaaggcttctggatacaccttcactagctatcatatacattgggtgcgccaggcccccggacaagggcttgagtggatcggatggatctaccctggcaatgttaacacagaatataatgagaagttcaagggcaaagccaccattaccgcggacgaatccacgaacacagcctacatggagctgagcagcctgagatctgaagacacggctgtgtattactgtgcgagggaggaaattacctacgctatggactactggggccagggaaccctggtcaccgtgtcctcaggcggaggtggaagcggagggggaggatctggcggcggaggaagcggaggcgacattcaaatgacccagagcccatccaccctgagcgcatctgtaggtgaccgggtcaccatcacttgtagatccagtcagagtattgtacacagtaatgggaacacctatttggaatggtatcagcagaaaccaggtaaagccccaaaattgctcatctacaaagtctctaacagatttagtggtgtaccagccaggttcagcggttccggaagtggtactgaattcaccctcacgatctcctctctccagccagatgatttcgccacttattactgttttcaaggttcacatgtgccgcgcacattcggtcagggtactaaagtagaagtcaaa

SEQ ID NO: 63 - protein, 269 aa, synthetic construct
MDMRVPAQLLGLLLLWLRGARCQVQLVQSGAEVKKPGSSVKVSCKASGYTFTSYHMHWVRQAPGQGLEWIGYIYPGNVNTEYNEKFKGKATLTADKSTNTAYMELSSLRSEDTAVYFCAREEITYAMDYWGQGTLVTVSSGGGGSGGGGSGGGGSGGDVQMTQSPSTLSASVGDRVTITCSSSQSIVHSNGNTYMEWYQQKPGKAPKLLIYKVSNRFSGVPDRFSGSGSGTEFTLTISSLQPDDFATYYCHQGSHVPRTFGQGTKVEVK

SEQ ID NO: 64 - DNA, 807 bp, synthetic construct
  source 1..807: mol_type = other DNA
atggacatgagggtccccgctcagctcctggggctcctgctactctggctccgaggtgccagatgtcaggtgcagctggtgcagtctggggctgaggtgaagaagcctgggtcctcagtgaaggtttcctgcaaggcttctggatacaccttcactagctatcatatgcattgggtgcgccaggcccccggacaagggcttgagtggatcggatacatctaccctggcaatgttaacacagaatataatgagaagttcaagggcaaagccacccttaccgcggacaaatccacgaacacagcctacatggagctgagcagcctgagatctgaagacacggctgtgtatttctgtgcgagggaggaaattacctacgctatggactactggggccagggaaccctggtcaccgtgtcctcaggcggaggtggaagcggagggggaggatctggcggcggaggaagcggaggcgacgttcaaatgacccagagcccatccaccctgagcgcatctgtaggtgaccgggtcaccatcacttgtagctccagtcagagtattgtacacagtaatgggaacacctatatggaatggtatcagcagaaaccaggtaaagccccaaaattgctcatctacaaagtctctaacagatttagtggtgtaccagacaggttcagcggttccggaagtggtactgaattcaccctcacgatctcctctctccagccagatgatttcgccacttattactgtcatcaaggttcacatgtgccgcgcacattcggtcagggtactaaagtagaagtcaaa

SEQ ID NO: 65 - protein, 247 aa, Mus musculus
DVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPRTSGGGTKLEIKGGGGSGGGGSGGGGSGGQVQLQQSGPELVKPGASVRISCKASGYTFTSYHIHWVKQRPGQGLEWIGWIYPGNVNTEYNEKFKGKATLTADKSSSTAYMHLSSLTSEDSAVYFCAREEITYAMDYWGQGTSVTVSS

SEQ ID NO: 66 - protein, 247 aa, Homo sapiens
QVQLVQSGAEVKKPGSSVKVSCKASGYTFTSYHIHWVRQAPGQGLEWMGWIYPGNVNTEYNEKFKGKATITADKSTSTAYMELSSLRSEDTAVYYCAREEITYAMDYWGQGTTVTVSSGGGGSGGGGSGGGGSGGEIVLTQSPGTLSLSPGERATLSCRSSQSIVHSNGNTYLEWYQQKPGQAPRLLIYKVSNRFSGIPDRFSGSGSGTDFTLTISRLEPEDFAVYYCFQGSHVPRTFGGGTKVEIK

SEQ ID NO: 67 - protein, 247 aa, Homo sapiens
QVQLVQSGAEVKKPGSSVKVSCKASGYTFTSYHIHWVRQAPGQGLEWMGWIYPGNVNTEYNEKFKGKATITADKSTSTAYMELSSLRSEDTAVYYCAREEITYAMDYWGQGTTVTVSSGGGGSGGGGSGGGGSGGDIVMTQTPLSLPVTPGEPASISCRSSQSIVHSNGNTYLEWYLQKPGQSPQLLIYKVSNRFSGVPDRFSGSGSGTDFTLKISRVEAEDVGVYYCFQGSHVPRTFGGGTKVEIK

SEQ ID NO: 68 - protein, 247 aa, Homo sapiens
EVQLVESGGGLVKPGGSLRLSCAASGYTFTSYHIHWVRQAPGKGLEWVGWIYPGNVNTEYNEKFKGRFTISRDDSKNTLYLQMNSLKTEDTAVYYCAREEITYAMDYWGQGTTVTVSSGGGGSGGGGSGGGGSGGDIQMTQSPSSLSASVGDRVTITCRSSQSIVHSNGNTYLEWYQQKPGKAPKLLIYKVSNRFSGVPSRFSGSGSGTDFTLTISSLQPEDFATYYCFQGSHVPRTFGGGTKVEIK

SEQ ID NO: 69 - protein, 247 aa, Homo sapiens
QVQLVQSGAEVKKPGSSVKVSCKASGYTFTSYHIHWVRQAPGQGLEWIGWIYPGNVNTEYNEKFKGKATITADESTNTAYMELSSLRSEDTAVYYCAREEITYAMDYWGQGTLVTVSSGGGGSGGGGSGGGGSGGDIQMTQSPSTLSASVGDRVTITCRSSQSIVHSNGNTYLEWYQQKPGKAPKLLIYKVSNRFSGVPARFSGSGSGTEFTLTISSLQPDDFATYYCFQGSHVPRTFGQGTKVEVK

SEQ ID NO: 70 - protein, 247 aa, Homo sapiens
QVQLVQSGAEVKKPGSSVKVSCKASGYTFTSYHMHWVRQAPGQGLEWIGYIYPGNVNTEYNEKFKGKATLTADKSTNTAYMELSSLRSEDTAVYFCAREEITYAMDYWGQGTLVTVSSGGGGSGGGGSGGGGSGGDVQMTQSPSTLSASVGDRVTITCSSSQSIVHSNGNTYMEWYQQKPGKAPKLLIYKVSNRFSGVPDRFSGSGSGTEFTLTISSLQPDDFATYYCHQGSHVPRTFGQGTKVEVK

SEQ ID NO: 71 - protein, 247 aa, Mus musculus
QVQLQQSGPELVKPGASVKMSCKASGYTFTSYHIQWVKQRPGQGLEWIGWIYPGDGSTQYNEKFKGKTTLTADKSSSTAYMLLSSLTSEDSAIYFCAREGTYYAMDYWGQGTSVTVSSGGGGSGGGGSGGGGSGGDVLMTQTPLSLPVSLGDQVSISCRSSQSIVHSNGNTYLEWYLQKPGQSPKLLIYKVSNRFSGVPDRFSGSGSGTDFTLKISRVEAEDLGVYYCFQGSHVPRTFGGGTKLEIK

SEQ ID NO: 72 - protein, 247 aa, Homo sapiens
QLQLQESGPGLVKPSETLSLTCTVSGYTFTSYHIQWIRQPPGKGLEWIGWIYPGDGSTQYNEKFKGRATISVDTSKNQFSLNLDSVSAADTAIYYCAREGTYYAMDYWGKGSTVTVSSGGGGSGGGGSGGGGSGGDIQMTQSPSSLSASVGDRVTITCRSSQSIVHSNGNTYLEWYQQKPGKAPKLLIYKVSNRFSGVPSRFSGSGSGTDFTFTISSLQPEDIATYYCFQGSHVPRTFGPGTKVDIK

SEQ ID NO: 73 - protein, 247 aa, Homo sapiens
EVQLVQSGAELKKPGSSVKVSCKASGYTFTSYHIQWVKQAPGQGLEWIGWIYPGDGSTQYNEKFKGKATLTVDKSTNTAYMELSSLRSEDTAVYYCAREGTYYAMDYWGQGTLVTVSSGGGGSGGGGSGGGGSGGDIQMTQSPSTLSASVGDRVTITCRSSQSIVHSNGNTYLEWYQQKPGKAPKLLIYKVSNRFSGVPSRFSGSGSGTDFTLTISSLQPDDFATYYCFQGSHVPRTFGQGTKVEVK

SEQ ID NO: 74 - protein, 247 aa, Homo sapiens
QVQLVQSGAEVKKPGSSVKVSCKASGYTFTSYHIQWVRQAPGQGLEWMGWIYPGDGSTQYNEKFKGRVTITADKSTSTAYMELSSLRSEDTAVYYCAREGTYYAMDYWGQGTTVTVSSGGGGSGGGGSGGGGSGGEIVLTQSPGTLSLSPGERATLSCRSSQSIVHSNGNTYLEWYQQKPGQAPRLLIYKVSNRFSGIPDRFSGSGSGTDFTLTISRLEPEDFAVYYCFQGSHVPRTFGGGTKVEIK

SEQ ID NO: 75 - protein, 247 aa, Homo sapiens
QVTLKQSGAEVKKPGSSVKVSCTASGYTFTSYHVSWVRQAPGQGLEWLGRIYPGDGSTQYNEKFKGKVTITADKSMDTSFMELTSLTSEDTAVYYCAREGTYYAMDLWGQGTLVTVSSGGGGSGGGGSGGGGSGGEIVLTQSPGTLSLSPGERATLSCRSSQSIVHSNGNTYLAWYQQKPGQAPRLLISKVSNRFSGVPDRFSGSGSGTDFTLTISRLEPEDFAVYYCQQGSHVPRTFGGGTKVEIK

SEQ ID NO: 76 - protein, 247 aa, Homo sapiens
QVQLVQSGAEVKKPGASVKVSCKASGYTFTSYHMHWVRQAPGQRLEWMGWIYPGDGSTQYNEKFKGKVTITRDTSASTAYMELSSLRSEDTAVYYCAREGTYYAMDYWGQGTLVTVSSGGGGSGGGGSGGGGSGGDIVMTQTPLSLPVTPGEPASISCRSSQSIVHSNGNTYLDWYLQKPGQSPQLLIYKVSNRFSGVPDRFSGSGSGTDFTLKISRVEAEDVGVYYCMQGSHVPRTFGGGTKVEIK

SEQ ID NO: 77 - protein, 16 aa, synthetic construct
RSSQSIVHSNGNTYLE

SEQ ID NO: 78 - protein, 12 aa, synthetic construct
KVSNRFSGVPDR

SEQ ID NO: 79 - protein, 12 aa, synthetic construct
ASGYTFTSYHIH

SEQ ID NO: 80 - protein, 12 aa, synthetic construct
ASGYTFTSYHIH

SEQ ID NO: 81 - protein, 18 aa, synthetic construct
WIYPGNVNTEYNEKFKGK

SEQ ID NO: 82 - protein, 9 aa, synthetic construct
EEITYAMDY

SEQ ID NO: 83 - protein, 12 aa, synthetic construct
KVSNRFSGVPAR

SEQ ID NO: 84 - protein, 16 aa, synthetic construct
SSSQSIVHSNGNTYME

SEQ ID NO: 85 - protein, 9 aa, synthetic construct
HQGSHVPRT

SEQ ID NO: 86 - protein, 12 aa, synthetic construct
ASGYTFTSYHMH

SEQ ID NO: 87 - protein, 18 aa, synthetic construct
YIYPGNVNTEYNEKFKGK

SEQ ID NO: 88 - protein, 16 aa, synthetic construct
RSSQSIVHSNGNTYLD

SEQ ID NO: 89 - protein, 9 aa, synthetic construct
MQGSHVPRT

SEQ ID NO: 90 - protein, 11 aa, synthetic construct
SGYTFTSYHMH

SEQ ID NO: 91 - protein, 17 aa, synthetic construct
WIYPGDGSTQYNEKFKG

SEQ ID NO: 92 - protein, 9 aa, synthetic construct
EGTYYAMDY

SEQ ID NO: 93 - protein, 27 aa, synthetic construct
FWVLVVVGGVLACYSLLVTVAFIIFWV

SEQ ID NO: 94 - protein, 21 aa, synthetic construct
IYIWAPLAGTCGVLLLSLVIT

SEQ ID NO: 95 - protein, 6 aa, synthetic construct
NLYAAV

SEQ ID NO: 96 - protein, 6 aa, synthetic construct
VTYAEV

SEQ ID NO: 97 - protein, 6 aa, synthetic construct
VTYAQL

SEQ ID NO: 98 - protein, 6 aa, synthetic construct
SIYATL

SEQ ID NO: 99 - protein, 23 aa, synthetic construct
VIGFRILLLKVAGFNLLMTLRLW

SEQ ID NO: 100 - protein, 23 aa, synthetic construct
TILYEILLGKATLYAVLVSALVL

SEQ ID NO: 101 - protein, 670 aa, synthetic construct
MTPILTVLICLGLSLGPRTHVQAGHLPKPTLWAEPGSVITQGSPVTLRCQGGQETQEYRLYREKKTALWITRIPQELVKKGQFPIPSITWEHAGRYRCYYGSDTAGRSESSDPLELVVTGAYIKPTLSAQPSPVVNSGGNVILQCDSQVAFDGFSLCKEGEDEHPQCLNSQPHARGSSRAIFSVGPVSPSRRWWYRCYAYDSNSPYEWSLPSDLLELLVLGVSKKPSLSVQPGPIVAPEETLTLQCGSDAGYNRFVLYKDGERDFLQLAGAQPQAGLSQANFTLGPVSRSYGGQYRCYGAHNLSSEWSAPSDPLDILIAGQFYDRVSLSVQPGPTVASGENVTLLCQSQGWMQTFLLTKEGAADDPWRLRSTYQSQKYQAEFPMGPVTSAHAGTYRCYGSQSSKPYLLTHPSDPLELVVSGPSGGPSSPTTGPTSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIHPSQEGPSPAVPSIYATLAIH

SEQ ID NO: 102 - protein, 253 aa, synthetic construct
YGSQSSKPYLLTHPSDPLELVVSGPSGGPSSPTTGPTSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 103 - protein, 20 aa, synthetic construct
YGSQSSKPYLLTHPSDPLEL

SEQ ID NO: 104 - protein, 233 aa, synthetic construct
VVSGPSGGPSSPTTGPTSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 105 - protein, 63 aa, synthetic construct
YGSQSSKPYLLTHPSDPLELVVSGPSGGPSSPTTGPTSTSGPEDQPLTPTGSDPQSGLGRHLG

SEQ ID NO: 106 - protein, 23 aa, synthetic construct
VVIGILVAVILLLLLLLLLFLIL

SEQ ID NO: 107 - protein, 167 aa, synthetic construct
RHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 108 - protein, 6 aa, synthetic construct
NLYAAV

SEQ ID NO: 109 - protein, 6 aa, synthetic construct
VTYAEV

SEQ ID NO: 110 - protein, 6 aa, synthetic construct
VTYAQL

SEQ ID NO: 111 - protein, 6 aa, synthetic construct
SIYATL

SEQ ID NO: 112 - protein, 35 aa, synthetic construct
NLYAAVKHTQPEDGVEMDTRSPHDEDPQAVTYAEV

SEQ ID NO: 113 - protein, 58 aa, synthetic construct
VTYAEVKHSRPRREMASPPSPLSGEFLDTKDRQAEEDRQMDTEAAASEAPQDVTYAQL

SEQ ID NO: 114 - protein, 36 aa, synthetic construct
VTYAQLHSLTLRREATEPPPSQEGPSPAVPSIYATL

SEQ ID NO: 115 - protein, 87 aa, synthetic construct
NLYAAVKHTQPEDGVEMDTRSPHDEDPQAVTYAEVKHSRPRREMASPPSPLSGEFLDTKDRQAEEDRQMDTEAAASEAPQDVTYAQL

SEQ ID NO: 116 - protein, 88 aa, synthetic construct
VTYAEVKHSRPRREMASPPSPLSGEFLDTKDRQAEEDRQMDTEAAASEAPQDVTYAQLHSLTLRREATEPPPSQEGPSPAVPSIYATL

SEQ ID NO: 117 - protein, 117 aa, synthetic construct
NLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATL

SEQ ID NO: 118 - protein, 20 aa, synthetic construct
YGSQSSKPYLLTHPSDPLEL

SEQ ID NO: 119 - protein, 43 aa, synthetic construct
VVSGPSGGPSSPTTGPTSTSGPEDQPLTPTGSDPQSGLGRHLG

SEQ ID NO: 120 - protein, 64 aa, synthetic construct
YGSQSSKPYLLTHPSDPLELVVSGPSGGPSSPTTGPTSTSGPEDQPLTPTGSDPQSGLGRHLGV

SEQ ID NO: 121 - protein, 44 aa, synthetic construct
VVSGPSGGPSSPTTGPTSTSGPEDQPLTPTGSDPQSGLGRHLGV

SEQ ID NO: 122 - protein, 67 aa, synthetic construct
AGSGGSGGSGGSPVPSTPPTPSPSTPPTPSPSGGSGNSSGSGGSPVPSTPPTPSPSTPPTPSPSASV

SEQ ID NO: 123 - protein, 75 aa, synthetic construct
AGSGGSGGSGGSPVPSTPPTNSSSTPPTPSPSPVPSTPPTNSSSTPPTPSPSPVPSTPPTNSSSTPPTPSPSASV

SEQ ID NO: 124 - protein, 85 aa, synthetic construct
VVSGPSGGPSSPTTGPTSTSGPEDQPLTPTGSDPQSGLGRHVVSGPSGGPSSPTTGPTSTSGPEDQPLTPTGSDPQSGLGRHLGV

SEQ ID NO: 125 - protein, 32 aa, synthetic construct
TTGPTSTSGPEDQPLTPTGSDPQSGLGRHLGV

SEQ ID NO: 126 - protein, 86 aa, synthetic construct
YGSQSSKPYLLTHPSDPLELVVSGPSGGPSSPTTGPTSTSGPEDQPLTPTGSDPQSGLGRHLGVVIGILVAVILLLLLLLLLFLIL

SEQ ID NO: 127 - protein, 190 aa, synthetic construct
VVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 128 - DNA, 762 bp, synthetic construct
  source 1..762: mol_type = other DNA
tacggctcacagagctccaaaccctacctgctgactcaccccagtgaccccctggagctcgtggtctcaggaccgtctgggggccccagctccccgacaacaggccccacctccacatctggccctgaggaccagcccctcacccccaccgggtcggatccccagagtggtctgggaaggcacctgggggttgtgatcggcatcttggtggccgtcatcctactgctcctcctcctcctcctcctcttcctcatcctccgacatcgacgtcagggcaaacactggacatcgacccagagaaaggctgatttccaacatcctgcaggggctgtggggccagagcccacagacagaggcctgcagtggaggtccagcccagctgccgatgcccaggaagaaaacctctatgctgccgtgaagcacacacagcctgaggatggggtggagatggacactcggagcccacacgatgaagacccccaggcagtgacgtatgccgaggtgaaacactccagacctaggagagaaatggcctctcctccttccccactgtctggggaattcctggacacaaaggacagacaggcggaagaggacaggcagatggacactgaggctgctgcatctgaagccccccaggatgtgacctacgcccagctgcacagcttgaccctcagacgggaggcaactgagcctcctccatcccaggaagggccctctccagctgtgcccagcatctacgccactctggccatccactag

SEQ ID NO: 129 - protein, 320 aa, synthetic construct
MSQEDIIRNIARHLAQVGDSMDRSIPPGEKTMLVLALLLAKKVASHTPSLLRDVFHTTVNFINQNLGSKKNSLALSLTADQMVSALLDAEPPILYSEYDPTRPFSEASMMGLLTNLADRELVHMINWAKRVPGFVDLTLHDQVHLLECAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEMLDAHRLHAPT

SEQ ID NO: 130 - DNA, 807 bp, synthetic construct
  source 1..807: mol_type = other DNA
atggacatgagggtccccgctcagctcctggggctcctgctactctggctccgaggtgccagatgtcaggtgcagctggtgcagtctggggctgaggtgaagaagcctgggtcctcagtgaaggtttcctgcaaggcttctggatacaccttcactagctatcatatacattgggtgcgccaggcccccggacaatgtcttgagtggatcggatggatctaccctggcaatgttaacacagaatataatgagaagttcaagggcaaagccaccattaccgcggacgaatccacgaacacagcctacatggagctgagcagcctgagatctgaagacacggctgtgtattactgtgcgagggaggaaattacctacgctatggactactggggccagggaaccctggtcaccgtgtcctcaggcggaggtggaagcggagggggaggatctggcggcggaggaagcggaggcgacattcaaatgacccagagcccatccaccctgagcgcatctgtaggtgaccgggtcaccatcacttgtagatccagtcagagtattgtacacagtaatgggaacacctatttggaatggtatcagcagaaaccaggtaaagccccaaaattgctcatctacaaagtctctaacagatttagtggtgtaccagccaggttcagcggttccggaagtggtactgaattcaccctcacgatctcctctctccagccagatgatttcgccacttattactgttttcaaggttcacatgtgccgcgcacattcggttgcggtactaaagtagaagtcaaa

SEQ ID NO: 131 - protein, 269 aa, synthetic construct
MDMRVPAQLLGLLLLWLRGARCQVQLVQSGAEVKKPGSSVKVSCKASGYTFTSYHIHWVRQAPGQCLEWIGWIYPGNVNTEYNEKFKGKATITADESTNTAYMELSSLRSEDTAVYYCAREEITYAMDYWGQGTLVTVSSGGGGSGGGGSGGGGSGGDIQMTQSPSTLSASVGDRVTITCRSSQSIVHSNGNTYLEWYQQKPGKAPKLLIYKVSNRFSGVPARFSGSGSGTEFTLTISSLQPDDFATYYCFQGSHVPRTFGCGTKVEVK

SEQ ID NO: 132 - DNA, 759 bp, synthetic construct
  source 1..759: mol_type = other DNA
tacggctcacagagctccaaaccctacctgctgactcaccccagtgaccccctggagctcgtggtctcaggaccgtctgggggccccagctccccgacaacaggccccacctccacatctggccctgaggaccagcccctcacccccaccgggtcggatccccagagtggtctgggaaggcacctgggggttgtgatcggcatcttggtggccgtcatcctactgctcctcctcctcctcctcctcttcctcatcctccgacatcgacgtcagggcaaacactggacatcgacccagagaaaggctgatttccaacatcctgcaggggctgtggggccagagcccacagacagaggcctgcagtggaggtccagcccagctgccgatgcccaggaagaaaacctctatgctgccgtgaagcacacacagcctgaggatggggtggagatggacactcggagcccacacgatgaagacccccaggcagtgacgtatgccgaggtgaaacactccagacctaggagagaaatggcctctcctccttccccactgtctggggaattcctggacacaaaggacagacaggcggaagaggacaggcagatggacactgaggctgctgcatctgaagccccccaggatgtgacctacgcccagctgcacagcttgaccctcagacgggaggcaactgagcctcctccatcccaggaagggccctctccagctgtgcccagcatctacgccactctggccatccac

SEQ ID NO: 133 - DNA, 66 bp, synthetic construct
  source 1..66: mol_type = other DNA
ggatccggagccaccaacttcagcctgctgaagcaggccggcgacgtggaggagaaccccggcccc

SEQ ID NO: 134 - DNA, 666 bp, synthetic construct; identical to SEQ ID NO: 54
  source 1..666: mol_type = other DNA

SEQ ID NO: 135 - DNA, 321 bp, synthetic construct
  source 1..321: mol_type = other DNA
cggaccgtggccgctcccagcgtgttcatcttcccccccagcgacgagcagcttaagagcggtaccgctagcgtggtgtgcctgctgaacaacttctacccccgggaggccaaggtgcagtggaaggtggacaacgccctgcagagcggcaacagccaggaaagcgtcaccgagcaggacagcaaggactccacctacagcctgagcagcaccctgaccctgagcaaggccgactacgagaagcacaaggtgtacgcctgcgaagtgacccaccagggcctgtccagccccgtgaccaagagcttcaaccggggcgagtgc

SEQ ID NO: 136 - DNA, 477 bp, synthetic construct
  source 1..477: mol_type = other DNA
tggggccaaggaacctcagtcaccgtgtcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcataccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 137 - protein, 159 aa, synthetic construct; identical to SEQ ID NO: 2

SEQ ID NO: 138 - DNA, 477 bp, synthetic construct
  source 1..477: mol_type = other DNA
tggggtcaaggtacctcagtcaccgtgtcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcataccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 139 - protein, 159 aa, synthetic construct; identical to SEQ ID NO: 2

SEQ ID NO: 140 - DNA, 477 bp, synthetic construct
  source 1..477: mol_type = other DNA
tggggccagggaaccctggtcaccgtgtcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcataccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 141 - DNA, 771 bp, synthetic construct
  source 1..771: mol_type = other DNA
atgcttctcctggtgacaagccttctgctctgtgagttaccacacccagcattcctcctgatcccagacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacacttctcctggtgacaagccttctgctctgtgagttaccacacccagcattcctcctgatcccagacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcaca

SEQ ID NO: 142 - protein, 257 aa, synthetic construct
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 143 - DNA, 1092 bp, synthetic construct
  source 1..1092: mol_type = other DNA
atgcttctcctggtgacaagccttctgctctgtgagttaccacacccagcattcctcctgatcccagacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacacttctcctggtgacaagccttctgctctgtgagttaccacacccagcattcctcctgatcccagacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacacggaccgtggccgctcccagcgtgttcatcttcccccccagcgacgagcagcttaagagcggtaccgctagcgtggtgtgcctgctgaacaacttctacccccgggaggccaaggtgcagtggaaggtggacaacgccctgcagagcggcaacagccaggaaagcgtcaccgagcaggacagcaaggactccacctacagcctgagcagcaccctgaccctgagcaaggccgactacgagaagcacaaggtgtacgcctgcgaagtgacccaccagggcctgtccagccccgtgaccaagagcttcaaccggggcgagtgc

SEQ ID NO: 144 - protein, 364 aa, synthetic construct
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 145 - DNA, 393 bp, synthetic construct
  source 1..393: mol_type = other DNA
atggacatgagggtccccgctcagctcctggggctcctgctactctggctccgaggtgccagatgtgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactac

SEQ ID NO: 146 - protein, 131 aa, synthetic construct
MDMRVPAQLLGLLLLWLRGARCEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDY

SEQ ID NO: 147 - DNA, 870 bp, synthetic construct
  source 1..870: mol_type = other DNA
atggacatgagggtccccgctcagctcctggggctcctgctactctggctccgaggtgccagatgtgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtgtcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcataccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 148 - protein, 290 aa, synthetic construct
MDMRVPAQLLGLLLLWLRGARCEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCTTTPAPRPPTPAPTIASQPLSLRPEACRPAAGGAVHTRGLDFACD

SEQ ID NO: 149 - DNA, 2340 bp, synthetic construct
  source 1..2340: mol_type = other DNA
atggacatgagggtccccgctcagctcctggggctcctgctactctggctccgaggtgccagatgtcaggtgcagctggtgcagtctggggctgaggtgaagaagcctgggtcctcagtgaaggtttcctgcaaggcttctggatacaccttcactagctatcatatacattgggtgcgccaggcccccggacaatgtcttgagtggatcggatggatctaccctggcaatgttaacacagaatataatgagaagttcaagggcaaagccaccattaccgcggacgaatccacgaacacagcctacatggagctgagcagcctgagatctgaagacacggctgtgtattactgtgcgagggaggaaattacctacgctatggactactggggccagggaaccctggtcaccgtgtcctcaggcggaggtggaagcggagggggaggatctggcggcggaggaagcggaggcgacattcaaatgacccagagcccatccaccctgagcgcatctgtaggtgaccgggtcaccatcacttgtagatccagtcagagtattgtacacagtaatgggaacacctatttggaatggtatcagcagaaaccaggtaaagccccaaaattgctcatctacaaagtctctaacagatttagtggtgtaccagccaggttcagcggttccggaagtggtactgaattcaccctcacgatctcctctctccagccagatgatttcgccacttattactgttttcaaggttcacatgtgccgcgcacattcggttgcggtactaaagtagaagtcaaatacggctcacagagctccaaaccctacctgctgactcaccccagtgaccccctggagctcgtggtctcaggaccgtctgggggccccagctccccgacaacaggccccacctccacatctggccctgaggaccagcccctcacccccaccgggtcggatccccagagtggtctgggaaggcacctgggggttgtgatcggcatcttggtggccgtcatcctactgctcctcctcctcctcctcctcttcctcatcctccgacatcgacgtcagggcaaacactggacatcgacccagagaaaggctgatttccaacatcctgcaggggctgtggggccagagcccacagacagaggcctgcagtggaggtccagcccagctgccgatgcccaggaagaaaacctctatgctgccgtgaagcacacacagcctgaggatggggtggagatggacactcggagcccacacgatgaagacccccaggcagtgacgtatgccgaggtgaaacactccagacctaggagagaaatggcctctcctccttccccactgtctggggaattcctggacacaaaggacagacaggcggaagaggacaggcagatggacactgaggctgctgcatctgaagccccccaggatgtgacctacgcccagctgcacagcttgaccctcagacgggaggcaactgagcctcctccatcccaggaagggccctctccagctgtgcccagcatctacgccactctggccatccacggatccggagccaccaacttcagcctgctgaagcaggccggcgacgtggaggagaaccccggccccatgcttctcctggtgacaagccttctgctctgtgagttaccacacccagcattcctcctgatcccagacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacacggaccgtggccgctcccagcgtgttcatcttcccccccagcgacgagcagcttaagagcggtaccgctagcgtggtgtgcctgctgaacaacttctacccccgggaggccaaggtgcagtggaaggtggacaacgccctgcagagcggcaacagccaggaaagcgtcaccgagcaggacagcaaggactccacctacagcctgagcagcaccctgaccctgagcaaggccgactacgagaagcacaaggtgtacgcctgcgaagtgacccaccagggcctgtccagccccgtgaccaagagcttcaaccggggcgagtgc

SEQ ID NO: 150 - protein, 780 aa, synthetic construct
MDMRVPAQLLGLLLLWLRGARCQVQLVQSGAEVKKPGSSVKVSCKASGYTFTSYHIHWVRQAPGQCLEWIGWIYPGNVNTEYNEKFKGKATITADESTNTAYMELSSLRSEDTAVYYCAREEITYAMDYWGQGTLVTVSSGGGGSGGGGSGGGGSGGDIQMTQSPSTLSASVGDRVTITCRSSQSIVHSNGNTYLEWYQQKPGKAPKLLIYKVSNRFSGVPARFSGSGSGTEFTLTISSLQPDDFATYYCFQGSHVPRTFGCGTKVEVKYGSQSSKPYLLTHPSDPLELVVSGPSGGPSSPTTGPTSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIHGSGATNFSLLKQAGDVEENPGPMLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 151 - DNA, 1536 bp, synthetic construct
  source 1..1536: mol_type = other DNA
atggacatgagggtccccgctcagctcctggggctcctgctactctggctccgaggtgccagatgtgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtgtcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcataccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttctgggtgctggtcgttgtgggcggcgtgctggcctgctacagcctgctggtgacagtggccttcatcatcttttgggtgaggagcaagcggagcagactgctgcacagcgactacatgaacatgaccccccggaggcctggccccacccggaagcactaccagccctacgcccctcccagggatttcgccgcctaccggagcaaacggggcagaaagaaactcctgtatatattcaaacaaccatttatgaggccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagcgtagaggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggactcagtacagccaccaaggacacctacgacgcccttcacatgcaggccctgccccctcgc

SEQ ID NO: 152 - protein, 512 aa, synthetic construct
MDMRVPAQLLGLLLLWLRGARCEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 153 - DNA, 135 bp, synthetic construct
  source 1..135: mol_type = other DNA
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 154 - DNA, 135 bp, synthetic construct
  source 1..135: mol_type = other DNA
accacgacgccagcgccgcgaccaccaacaccggcgcccaccatcgcgtcgcagcccctgtccctgcgcccagaggcgagccggccagcggcggggggcgcagtgcacacgagggggctggacttcgccagtgat

SEQ ID NO: 155 - DNA, 90 bp, synthetic construct
  source 1..90: mol_type = other DNA
gtgggcatcattgctggcctggttctctttggagctgtgatcactggagctgtggtcgctgccgtgatgtggaggagcaagcggagcaga

SEQ ID NO: 156 - DNA, 288 bp, synthetic construct
  source 1..288: mol_type = other DNA
gacatgagggtccccgctcagctcctggggctcctgctactctggctccgaggtgccagatgtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatgtgggcatcattgctggcctggttctctttggagctgtgatcactggagctgtggtcgctgccgtgatgtggaggagcaagcggagcaga

SEQ ID NO: 157 - protein, 96 aa, synthetic construct
DMRVPAQLLGLLLLWLRGARCTTTPAPRPPTPAPTIASQPLSLRPEACRPAAGGAVHTRGLDFACDVGIIAGLVLFGAVITGAVVAAVMWRSKRSR

SEQ ID NO: 158 - DNA, 567 bp, synthetic construct
  source 1..567: mol_type = other DNA
ctgctgcacagcgactacatgaacatgaccccccggaggcctggccccacccggaagcactaccagccctacgcccctcccagggatttcgccgcctaccggagcaaacggggcagaaagaaactcctgtatatattcaaacaaccatttatgaggccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagcgtagaggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggactcagtacagccaccaaggacacctacgacgcccttcacatgcaggccctgccccctcgc

SEQ ID NO: 159 - protein, 189 aa, synthetic construct; identical to SEQ ID NO: 12

SEQ ID NO: 160 - DNA, 2490 bp, synthetic construct
  source 1..2490: mol_type = other DNA
atggacatgagggtccccgctcagctcctggggctcctgctactctggctccgaggtgccagatgtgatgttttgatgacccaaactccactctccctgcctgtcagtcttggagatcaagcctccatctcttgcagatctagtcagagcattgtacatagtaatggaaacacctatttagaatggtacctgcagaaaccaggccagtctccaaagctcctgatctacaaagtttccaaccgattttctggggtcccagacaggttcagtggcagtggatcagggacagatttcacactcaagatcagtagagtggaggctgaggatctgggagtttattactgctttcaaggttcacatgttcctcggacgtccggtggaggcaccaagctggaaatcaaaggcggaggtggaagcggagggggaggatctggcggcggaggaagcggaggccaggtccagctgcagcagtctggacctgagctggtgaagcctggggcttcagtgaggatatcctgcaaggcttctggctacaccttcacaagttaccatatacattgggtgaagcagaggcctggacagggacttgagtggattggatggatttatcctggaaatgttaatactgagtacaatgagaagttcaagggcaaggccacactgactgcagacaaatcgtccagcacagcctacatgcacctcagcagcctgacctctgaggactctgcggtctatttctgtgccagagaggagattacctatgctatggactactggggtcaaggaacctcagtcaccgtgtcctcatacggctcacagagctccaaaccctacctgctgactcaccccagtgaccccctggagctcgtggtctcaggaccgtctgggggccccagctccccgacaacaggccccacctccacatctggccctgaggaccagcccctcacccccaccgggtcggatccccagagtggtctgggaaggcacctgggggttgtgatcggcatcttggtggccgtcatcctactgctcctcctcctcctcctcctcttcctcatcctccgacatcgacgtcagggcaaacactggacatcgacccagagaaaggctgatttccaacatcctgcaggggctgtggggccagagcccacagacagaggcctgcagtggaggtccagcccagctgccgatgcccaggaagaaaacctctatgctgccgtgaagcacacacagcctgaggatggggtggagatggacactcggagcccacacgatgaagacccccaggcagtgacgtatgccgaggtgaaacactccagacctaggagagaaatggcctctcctccttccccactgtctggggaattcctggacacaaaggacagacaggcggaagaggacaggcagatggacactgaggctgctgcatctgaagccccccaggatgtgacctacgcccagctgcacagcttgaccctcagacgggaggcaactgagcctcctccatcccaggaagggccctctccagctgtgcccagcatctacgccactctggccatccacggatccggagccaccaacttcagcctgctgaagcaggccggcgacgtggaggagaaccccggccccatggacatgagggtccccgctcagctcctggggctcctgctactctggctccgaggtgccagatgtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatgtgggcatcattgctggcctggttctctttggagctgtgatcactggagctgtggtcgctgccgtgatgtggaggagcaagcggagcagactgctgcacagcgactacatgaacatgaccccccggaggcctggccccacccggaagcactaccagccctacgcccctcccagggatttcgccgcctaccggagcaaacggggcagaaagaaactcctgtatatattcaaacaaccatttatgaggccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagcgtagaggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggactcagtacagccaccaaggacacctacgacgcccttcacatgcaggccctgccccctcgc

SEQ ID NO: 161 - protein, 830 aa, synthetic construct
MDMRVPAQLLGLLLLWLRGARCDVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPRTSGGGTKLEIKGGGGSGGGGSGGGGSGGQVQLQQSGPELVKPGASVRISCKASGYTFTSYHIHWVKQRPGQGLEWIGWIYPGNVNTEYNEKFKGKATLTADKSSSTAYMHLSSLTSEDSAVYFCAREEITYAMDYWGQGTSVTVSSYGSQSSKPYLLTHPSDPLELVVSGPSGGPSSPTTGPTSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIHGSGATNFSLLKQAGDVEENPGPMDMRVPAQLLGLLLLWLRGARCTTTPAPRPPTPAPTIASQPLSLRPEACRPAAGGAVHTRGLDFACDVGIIAGLVLFGAVITGAVVAAVMWRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 162 - DNA, 2490 bp, synthetic construct
  source 1..2490: mol_type = other DNA
atggacatgagggtccccgctcagctcctggggctcctgctactctggctccgaggtgccagatgtgatgttttgatgacccaaactccactctccctgcctgtcagtcttggagatcaagcctccatctcttgcagatctagtcagagcattgtacatagtaatggaaacacctatttagaatggtacctgcagaaaccaggccagtctccaaagctcctgatctacaaagtttccaaccgattttctggggtcccagacaggttcagtggcagtggatcagggacagatttcacactcaagatcagtagagtggaggctgaggatctgggagtttattactgctttcaaggttcacatgttcctcggacgtccggtggaggcaccaagctggaaatcaaaggcggaggtggaagcggagggggaggatctggcggcggaggaagcggaggccaggtccagctgcagcagtctggacctgagctggtgaagcctggggcttcagtgaggatatcctgcaaggcttctggctacaccttcacaagttaccatatacattgggtgaagcagaggcctggacagggacttgagtggattggatggatttatcctggaaatgttaatactgagtacaatgagaagttcaagggcaaggccacactgactgcagacaaatcgtccagcacagcctacatgcacctcagcagcctgacctctgaggactctgcggtctatttctgtgccagagaggagattacctatgctatggactactggggtcaaggaacctcagtcaccgtgtcctcatacggctcacagagctccaaaccctacctgctgactcaccccagtgaccccctggagctcgtggtctcaggaccgtctgggggccccagctccccgacaacaggccccacctccacatctggccctgaggaccagcccctcacccccaccgggtcggatccccagagtggtctgggaaggcacctgggggttgtgatcggcatcttggtggccgtcatcctactgctcctcctcctcctcctcctcttcctcatcctccgacatcgacgtcagggcaaacactggacatcgacccagagaaaggctgatttccaacatcctgcaggggctgtggggccagagcccacagacagaggcctgcagtggaggtccagcccagctgccgatgcccaggaagaaaacctctatgctgccgtgaagcacacacagcctgaggatggggtggagatggacactcggagcccacacgatgaagacccccaggcagtgacgtatgccgaggtgaaacactccagacctaggagagaaatggcctctcctccttccccactgtctggggaattcctggacacaaaggacagacaggcggaagaggacaggcagatggacactgaggctgctgcatctgaagccccccaggatgtgacctacgcccagctgcacagcttgaccctcagacgggaggcaactgagcctcctccatcccaggaagggccctctccagctgtgcccagcatctacgccactctggccatccacggatccggagccaccaacttcagcctgctgaagcaggccggcgacgtggaggagaaccccggccccatggacatgagggtccccgctcagctcctggggctcctgctactctggctccgaggtgccagatgtaccacgacgccagcgccgcgaccaccaacaccggcgcccaccatcgcgtcgcagcccctgtccctgcgcccagaggcgagccggccagcggcggggggcgcagtgcacacgagggggctggacttcgccagtgatgtgggcatcattgctggcctggttctctttggagctgtgatcactggagctgtggtcgctgccgtgatgtggaggagcaagcggagcagactgctgcacagcgactacatgaacatgaccccccggaggcctggccccacccggaagcactaccagccctacgcccctcccagggatttcgccgcctaccggagcaaacggggcagaaagaaactcctgtatatattcaaacaaccatttatgaggccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagcgtagaggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggactcagtacagccaccaaggacacctacgacgcccttcacatgcaggccctgccccctcgc

SEQ ID NO: 163 - protein, 830 aa, synthetic construct
MDMRVPAQLLGLLLLWLRGARCDVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPRTSGGGTKLEIKGGGGSGGGGSGGGGSGGQVQLQQSGPELVKPGASVRISCKASGYTFTSYHIHWVKQRPGQGLEWIGWIYPGNVNTEYNEKFKGKATLTADKSSSTAYMHLSSLTSEDSAVYFCAREEITYAMDYWGQGTSVTVSSYGSQSSKPYLLTHPSDPLELVVSGPSGGPSSPTTGPTSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIHGSGATNFSLLKQAGDVEENPGPMDMRVPAQLLGLLLLWLRGARCTTTPAPRPPTPAPTIASQPLSLRPEASRPAAGGAVHTRGLDFASDVGIIAGLVLFGAVITGAVVAAVMWRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 164 - DNA, 1021 bp, synthetic construct
  source 1..1021: mol_type = other DNA
atgcttctcctggtgacaagccttctgctctgtgagttaccacacccagcattcctcctgatcccagacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggcggaggtggaagcggagggggaggatctggcggcggaggaagcggaggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtgtcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatgtgggcatcattgctggcctggttctctttggagctgtgatcactggagctgtggtcgctgccgtgatgtggaggagcaagcggagca

SEQ ID NO: 165 - protein, 340 aa, synthetic construct
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSGGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDVGIIAGLVLFGAVITGAVVAAVMWRSKRS

SEQ ID NO: 166 - DNA, 138 bp, synthetic construct
  source 1..138: mol_type = other DNA
agtgatggcagcctgactgatacactccaagcggagacagaccaactagaagatgagaagtctgctttgcagaccgagattgccaacctgctgaaggagaaggaaaaactagagttcatcctggcagctcacggatcc

SEQ ID NO: 167 - DNA, 137 bp, synthetic construct
  source 1..137: mol_type = other DNA
agtgatggaagcagaatcgcaagactggaagagaaggtaaagacattgaaggcgcagaatagcgagctggcttcaacggctaatatgttgagggagcaggtggctcagttgaagcagaaggtgatgaaccatggctc

SEQ ID NO: 168 - DNA, 129 bp, synthetic construct
  source 1..129: mol_type = other DNA
accacgacgccagcgccgcgaccaccaacaccggcgcccaccatcgcgtcgcagcccctgtccctgcgcccagaggcgagccggccagcggcggggggcgcagtgcacacgagggggctggacttcgcc

SEQ ID NO: 169 - protein, 43 aa, synthetic construct
TTTPAPRPPTPAPTIASQPLSLRPEASRPAAGGAVHTRGLDFA

SEQ ID NO: 170 - DNA, 267 bp, synthetic construct
  source 1..267: mol_type = other DNA
accacgacgccagcgccgcgaccaccaacaccggcgcccaccatcgcgtcgcagcccctgtccctgcgcccagaggcgagccggccagcggcggggggcgcagtgcacacgagggggctggacttcgccagtgatggcagcctgactgatacactccaagcggagacagaccaactagaagatgagaagtctgctttgcagaccgagattgccaacctgctgaaggagaaggaaaaactagagttcatcctggcagctcacggatcc

SEQ ID NO: 171 - DNA, 266 bp, synthetic construct
  source 1..266: mol_type = other DNA
accacgacgccagcgccgcgaccaccaacaccggcgcccaccatcgcgtcgcagcccctgtccctgcgcccagaggcgagccggccagcggcggggggcgcagtgcacacgagggggctggacttcgccagtgatggaagcagaatcgcaagactggaagagaaggtaaagacattgaaggcgcagaatagcgagctggcttcaacggctaatatgttgagggagcaggtggctcagttgaagcagaaggtgatgaaccatggctc

SEQ ID NO: 172 - DNA, 85 bp, synthetic construct
  source 1..85: mol_type = other DNA
agtcggcatcattgctggcctggttctctttggagctgtgatcactggagctgtggtcgctgccgtgatgtggaggagcaagcgg

SEQ ID NO: 173 - protein, 28 aa, synthetic construct
SRHHCWPGSLWSCDHWSCGRCRDVEEQA

SEQ ID NO: 174 - DNA, 2181 bp, synthetic construct
  source 1..2181: mol_type = other DNA
atgcttctcctggtgacaagccttctgctctgtgagttaccacacccagcattcctcctgatcccagacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggcggaggtggaagcggagggggaggatctggcggcggaggaagcggaggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtgtcctcaaccacgacgccagcgccgcgaccaccaacaccggcgcccaccatcgcgtcgcagcccctgtccctgcgcccagaggcgagccggccagcggcggggggcgcagtgcacacgagggggctggacttcgccagtgatggcagcctgactgatacactccaagcggagacagaccaactagaagatgagaagtctgctttgcagaccgagattgccaacctgctgaaggagaaggaaaaactagagttcatcctggcagctcacggatccgtgggcatcattgctggcctggttctctttggagctgtgatcactggagctgtggtcgctgccgtgatgtggaggagcaagcggagcagaggatccggagccaccaacttcagcctgctgaagcaggccggcgacgtggaggagaaccccggccccatgagtcaagaagacatcatccggaatattgccaggcacctcgcccaggtcggggacagcatggaccgtagcatccctccgggcgagaagaccatgctggtgctggccctgctgctggccaagaaggtggccagtcacacgccgtccttgctccgtgatgtctttcacacaacagtgaattttattaaccagaacctaggaagcaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggactcgtctggcgctccatggagcacccagggaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggagatgctggacgcccaccgcctacatgcgcccacc

SEQ ID NO: 175 - protein, 727 aa, synthetic construct
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSGGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEASRPAAGGAVHTRGLDFASDGSLTDTLQAETDQLEDEKSALQTEIANLLKEKEKLEFILAAHGSVGIIAGLVLFGAVITGAVVAAVMWRSKRSRGSGATNFSLLKQAGDVEENPGPMSQEDIIRNIARHLAQVGDSMDRSIPPGEKTMLVLALLLAKKVASHTPSLLRDVFHTTVNFINQNLGSKKNSLALSLTADQMVSALLDAEPPILYSEYDPTRPFSEASMMGLLTNLADRELVHMINWAKRVPGFVDLTLHDQVHLLECAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEMLDAHRLHAPT

SEQ ID NO: 176 - DNA, 1989 bp, synthetic construct
  source 1..1989: mol_type = other DNA
atggacatgagggtccccgctcagctcctggggctcctgctactctggctccgaggtgccagatgtaccacgacgccagcgccgcgaccaccaacaccggcgcccaccatcgcgtcgcagcccctgtccctgcgcccagaggcgagccggccagcggcggggggcgcagtgcacacgagggggctggacttcgccagtgatggaagcagaatcgcaagactggaagagaaggtaaagacattgaaggcgcagaatagcgagctggcttcaacggctaatatgttgagggagcaggtggctcagttgaagcagaaggtgatgaaccatggctcagtcggcatcattgctggcctggttctctttggagctgtgatcactggagctgtggtcgctgccgtgatgtggaggagcaagcggagcagactgctgcacagcgactacatgaacatgaccccccggaggcctggccccacccggaagcactaccagccctacgcccctcccagggatttcgccgcctaccggagcaaacggggcagaaagaaactcctgtatatattcaaacaaccatttatgaggccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagcgtagaggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggactcagtacagccaccaaggacacctacgacgcccttcacatgcaggccctgccccctcgcggatccggagccaccaacttcagcctgctgaagcaggccggcgacgtggaggagaaccccggccccatgaccagcaaggtgtacgatcccgagcagaggaagcgcatgatcacaggccctcagtggtgggccaggtgcaagcagatgaacgtgctggatagctttatcaattactatgactccgagaagcacgccgagaacgccgtgatcttcctgcacggcaatgccgccagctcctacctgtggaggcacgtggtgccccacatcgagcctgtggcaaggtgtatcatccccgatctgatcggcatgggcaagtctggcaagagcggcaacggctcctatcgcctgctggaccactacaagtatctgaccgcctggttcgagctgctgaatctgcctaagaagatcatctttgtgggacacgactggggagcatgcctggcattccactactcttatgagcaccaggataagatcaaggccatcgtgcacgccgagtctgtggtggacgtgatcgagagctgggacgagtggcccgacatcgaggaggacatcgccctgatcaagagcgaggagggcgagaagatggtgctggagaacaatttctttgtggagaccatgctgccctccaagatcatgcggaagctggagcctgaggagttcgccgcctacctggagcccttcaaggagaagggcgaggtgcggagaccaacactgtcttggccaagagagatccccctggtgaagggcggcaagcccgatgtggtgcagatcgtgcggaactacaatgcctatctgagagccagcgacgatctgcctaagatgtttatcgagtccgacccaggcttcttttctaacgccatcgtggagggcgccaagaagttccctaatacagagtttgtgaaggtgaagggcctgcacttttcccaggaggacgccccagatgagatgggcaagtatatcaagtctttcgtggagcgggtgctgaagaacgagcag

SEQ ID NO: 177 - protein, 663 aa, synthetic construct
MDMRVPAQLLGLLLLWLRGARCTTTPAPRPPTPAPTIASQPLSLRPEASRPAAGGAVHTRGLDFASDGSRIARLEEKVKTLKAQNSELASTANMLREQVAQLKQKVMNHGSVGIIAGLVLFGAVITGAVVAAVMWRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMTSKVYDPEQRKRMITGPQWWARCKQMNVLDSFINYYDSEKHAENAVIFLHGNAASSYLWRHVVPHIEPVARCIIPDLIGMGKSGKSGNGSYRLLDHYKYLTAWFELLNLPKKIIFVGHDWGACLAFHYSYEHQDKIKAIVHAESVVDVIESWDEWPDIEEDIALIKSEEGEKMVLENNFFVETMLPSKIMRKLEPEEFAAYLEPFKEKGEVRRPTLSWPREIPLVKGGKPDVVQIVRNYNAYLRASDDLPKMFIESDPGFFSNAIVEGAKKFPNTEFVKVKGLHFSQEDAPDEMGKYIKSFVERVLKNEQ